(19) United States
(12) Patent Application Publication
(10) Pub. No.: US 2008/0206737 A1
(43) Pub. Date: Aug. 28, 2008

(54) EXPRESSION QUANTIFICATION USING MASS SPECTROMETRY

(76) Inventor: Christie L. Hunter, San Mateo, CA (US)

Correspondence Address: AMBION, 2130 WOODWARD STREET, AUSTIN, TX, 78744-1832, US

(21) Appl. No.: 11/757,620

(22) Filed: Jun. 4, 2007

Related U.S. Application Data
(63) Continuation-in-part of application No. 11/441,457, filed on May 25, 2006.
Application No. 11/441,457 is a continuation-in-part of application No. 11/134,850, filed on May 19, 2005.
(60) Provisional application No. 60/572,826, filed on May 19, 2004.

(51) Int. Cl. G01N 33/68; C12Q 1/00

(52) U.S. Cl. 435/4; 436/86

(57) ABSTRACT

In various aspects, the present teachings provide systems, methods, assays and kits for the absolute quantitation of protein expression. In various aspects, the present teachings provide methods of determining the concentration of about the top forty-one proteins present in human plasma. In various aspects, the present teachings provide methods of determining the absolute concentration of one or more proteins using standard samples of signature protein fragments and parent-daughter ion transition monitoring (PDITM). In various embodiments, the absolute concentration of multiple isoforms of a biomolecule in a sample, multiple proteins in a biological process, a combination of multiple samples, or combinations thereof, can be determined in a multiplex fashion using the present teachings. In various aspects, provided are methods of assessing the state of a biological system including, but not limited to, the disease state of an animal.

### CROSS-REFERENCE TO RELATED APPLICATIONS

The present application is a continuation-in-part of and claims the benefit of and priority to copending U.S. application Ser. No. 11/441,457, entitled “Expression Quantification Using Mass Spectrometry”, filed May 25, 2006, and claims the benefit of and priority to U.S. application Ser. No. 11/134,850, entitled “Expression Quantification Using Mass Spectrometry”, filed May 19, 2005, which claims the benefit of and priority to U.S. Provisional Application No. 60/572,826, entitled “Expression Quantification Using Mass Spectrometry”, filed May 19, 2004, the entire disclosures of all of which are herein incorporated by reference.

### INTRODUCTION

Understanding protein expression is important to understanding biological systems. Unlike mRNA, which only acts as a disposable messenger, proteins implement almost all controlled biological functions and, as a result, are integral to such functions as normal cell activity, disease processes, and drug responses. However, protein expression is not reliably predictable. First, protein expression is not predictable from mRNA expression maps because mRNA transcript levels are not always strongly correlated with protein levels. Second, proteins are dynamically modified in biological systems by environmental factors in ways which are not predictable from genetic information.

Further, the function of a protein can be modulated by its abundance and its degree of modifications. Changes in protein expression (or concentration) and the extent of protein modifications can have a great influence on the activity, for example, of intracellular substrate degradation processes, biosynthetic pathways, the cell cycle, or the function of a single cell in a whole organism. As a result, changes in protein concentration could, for example, provide information on a biological state at the molecular level, on potential drug targets, the toxicity of a drug, the possibility of a drug forming a dangerous metabolite, and serve as biomarkers for certain disease states or markers that predict the likelihood of a positive response to a specialized drug therapy.

In general, approaches to quantifying protein expression fall into two categories, relative quantitation and absolute quantitation. Although absolute quantitation typically provides more information than relative quantitation, it has traditionally been more difficult to implement.

### SUMMARY

The present teachings provide systems, methods, assays and kits for the absolute quantitation of protein expression. In various aspects, methods of determining the absolute concentration of one or more isoforms of a protein using standard samples of signature protein fragments and parent-daughter ion transition monitoring (PDITM) are provided. In various embodiments, the protein isoforms comprise one or more isoenzymes, one or more isomers, or combinations thereof. In various embodiments, the absolute concentration of multiple isoforms of a biomolecule in a sample, multiple proteins in a biological process (e.g., to cover families of biomarkers, biological pathways, etc.), a combination of multiple samples, or combinations thereof, can be determined in a multiplex fashion, for example, from a single loading of the sample (or combined samples) onto a chromatographic column followed by PDITM.

The term “parent-daughter ion transition monitoring” or “PDITM” refers to, for example, a measurement using mass spectrometry whereby the transmitted mass-to-charge (m/z) range of a first mass separator (often referred to as the first dimension of mass spectrometry) is selected to transmit a molecular ion (often referred to as “the parent ion” or “the precursor ion”) to an ion fragmentor (e.g., a collision cell, photodissociation region, etc.) to produce fragment ions (often referred to as “daughter ions”) and the transmitted m/z range of a second mass separator (often referred to as the second dimension of mass spectrometry) is selected to transmit one or more daughter ions to a detector which measures the daughter ion signal. The combination of parent ion and daughter ion masses monitored can be referred to as the “parent-daughter ion transition” monitored. The daughter ion signal at the detector for a given parent ion-daughter ion combination monitored can be referred to as the “parent-daughter ion transition signal”. In the present teachings, where the parent ion is a signature peptide and the ion signal of a diagnostic daughter ion is measured, the diagnostic daughter ion signal at the detector for a given signature peptide ion-diagnostic daughter ion combination monitored can be referred to as the “signature peptide-diagnostic daughter ion transition signal”.

For example, one embodiment of parent-daughter ion transition monitoring is multiple reaction monitoring (MRM) (also referred to as selective reaction monitoring). In various embodiments of MRM, the monitoring of a given parent-daughter ion transition comprises using as the first mass separator a first quadrupole parked on the parent ion m/z of interest to transmit the parent ion of interest and using as a second mass separator a second quadrupole parked on the daughter ion m/z of interest to transmit daughter ions of interest. In various embodiments, a PDITM can be performed, for example, by parking the first mass separator on parent ion m/z of interest to transmit parent ions and scanning the second mass separator over a m/z range including the m/z value of the daughter ion of interest and, e.g., extracting an ion intensity profile from the spectra.

For example, a tandem mass spectrometer (MS/MS) instrument or, more generally, a multidimensional mass spectrometer (MSⁿ) instrument, can be used to perform PDITM, e.g., MRM.

In various embodiments, one or more proteins of interest can be used for, e.g., normalization of diagnostic daughter ion signals, normalization of the concentration of a protein in a first sample relative the concentration in a second sample (e.g., normalize a concentration ratio), evaluation of data reliability, evaluation of starting sample amount across samples, or combinations thereof. Herein, such proteins are referred to as normalization proteins. Accordingly, in various embodiments, the term “normalization protein” refers to a protein which is anticipated to have substantially the same concentration in two or more of the two or more samples, is anticipated to have a concentration that is not substantially affected by treatment of a sample with a chemical agent, or both. For example, in various embodiments, a protein of interest can be a protein known to have substantially the same concentration between samples. In various embodiments, changes in the signal level of a signature peptide of a normalization protein can be used to normalize the signal levels of the signature peptides of one or more proteins of interest. In various embodiments, differences in the signature peptide signal level of a normalization protein between two samples can be used to evaluate data reliability. For example, where the signature peptide signal associated with a normalization protein varies by a significant amount between samples, the data associated with one or both of these samples is excluded as unreliable. In various embodiments, it is not necessary to determine the absolute concentration of a normalization protein because, e.g., the ratio of the signature peptide signal associated with a normalization protein in one sample to that in another sample can be used to normalize the signal levels of the signature peptides of one or more proteins of interest, the concentration of a protein of interest in one sample relative to that in another sample, evaluation of starting sample amount across samples, evaluate the reliability of data, or combinations thereof.

In various embodiments, provided are methods for determining the concentration of one or more proteins of interest in one or more samples, comprising the steps of: (a) providing a standard sample for each of one or more proteins of interest, each standard sample comprising a signature peptide for the corresponding protein of interest; (b) selecting one or more signature peptide-diagnostic daughter ion transitions for at least one signature peptide of each standard sample; (c) generating a concentration curve for each selected signature peptide-diagnostic daughter ion transition; (d) labeling the one or more proteins of interest in the one or more samples with a chemical moiety; (e) loading at least a portion of each of the one or more labeled samples on a chromatographic column; (f) directing at least a portion of the eluent from the chromatographic column to a mass spectrometry system; (g) measuring the signature peptide-diagnostic daughter ion transition signal of one or more of the selected signature peptide-diagnostic daughter ion transitions; and (h) determining the absolute concentration of a protein of interest in one or more of the labeled samples based at least on a comparison of the measured signature peptide-diagnostic daughter ion transition signal corresponding to the protein of interest to the concentration curve for that signature peptide-diagnostic daughter ion transition. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

In various embodiments, provided are methods for determining the concentration of one or more proteins of interest in one or more samples, comprising the steps of: (a) providing a standard sample comprising a signature peptide for each corresponding protein of interest; (b) selecting one or more signature peptide-diagnostic daughter ion transitions for each signature peptide; (c) labeling the one or more proteins of interest in the one or more samples with a chemical moiety to produce one or more labeled samples; (d) labeling one or more standard samples with a chemical moiety; (e) combining, to produce a combined sample, at least a portion of the one or more labeled standard samples with at least a portion of one or more labeled samples, the labeled samples being labeled with a different chemical moiety than the one or more labeled standard samples combined therewith; (f) loading at least a portion of each of the one or more combined samples on a chromatographic column; (g) directing at least a portion of the eluent from the chromatographic column to a mass spectrometry system; (h) measuring the signature peptide-diagnostic daughter ion transition signal of one or more of the selected signature peptide-diagnostic daughter ion transitions; and (i) determining the absolute concentration of a protein of interest in one or more of the labeled samples based at least on a comparison of the measured signature peptide-diagnostic daughter ion transition signal for the protein of interest to the measured signature peptide-diagnostic daughter ion transition signal for a labeled standard sample. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

In various embodiments, provided are methods for determining the concentration of one or more proteins of interest in one or more samples, comprising the steps of: (a) providing a standard sample for each of one or more proteins of interest, each standard sample comprising a signature peptide for the corresponding protein of interest; (b) selecting one or more signature peptide-diagnostic daughter ion transitions for at least one signature peptide of each standard sample; (c) generating a concentration curve for each selected signature peptide-diagnostic daughter ion transition; (d) labeling the one or more proteins of interest in the one or more samples with a chemical moiety; (e) labeling one or more standard samples with a chemical moiety; (f) combining, to produce a combined sample, at least a portion of the one or more labeled standard samples with at least a portion of one or more labeled samples, the labeled sampled being labeled with a different chemical moiety than the one or more labeled standard samples combined therewith; (g) loading at least a portion of each of the one or more combined samples on a chromatographic column; (h) directing at least a portion of the eluent from the chromatographic column to a mass spectrometry system; (i) measuring the signature peptide-diagnostic daughter ion transition signal of one or more of the selected signature peptide-diagnostic daughter ion transitions; and (j) determining the absolute concentration of a protein of interest in one or more of the labeled samples based at least on a comparison of the measured signature peptide-diagnostic daughter ion transition signal corresponding to the protein of interest to one or more of the concentration curve for that signature peptide-diagnostic daughter ion transition and the measured signature peptide-diagnostic daughter ion transition signal for a labeled standard sample. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

In various embodiments, provided are methods for determining the concentration of one or more proteins of interest in two or more samples, comprising the steps of: (a) providing a standard sample for each of one or more proteins of interest, each standard sample comprising a signature peptide for the corresponding protein of interest; (b) selecting one or more signature peptide-diagnostic daughter ion transitions for at least one signature peptide of each standard sample; (c) generating a concentration curve for each selected diagnostic daughter ion; (d) labeling the one or more proteins of interest in two or more samples with different chemical moieties for each sample, the two or more samples thereby being differentially labeled; (e) combining at least a portion of the differentially labeled samples to produce a combined sample; (f) loading at least a portion of the combined sample on a chromatographic column; (g) directing at least a portion of the eluent from the chromatographic column to a mass spectrometry system; (h) measuring the signature peptide-diagnostic daughter ion transition signal of one or more of the selected signature peptide-diagnostic daughter ion transitions; and (i) determining the absolute concentration of a protein of interest in one or more of the differentially labeled samples based at least on a comparison of the measured signature peptide-diagnostic daughter ion transition signal for the protein of interest to the concentration curve for that signature peptide-diagnostic daughter ion transition. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

In various embodiments, provided are methods for determining the concentration of one or more proteins of interest in two or more samples, comprising the steps of: (a) providing a standard sample for each of one or more proteins of interest, each standard sample comprising a signature peptide for the corresponding protein of interest; (b) selecting one or more signature peptide-diagnostic daughter ion transitions for at least one signature peptide of each standard sample; (c) labeling the one or more proteins of interest in two or more samples with different chemical moieties for each sample, the two or more samples thereby being differentially labeled; (d) labeling one or more standard samples with a chemical moiety; (e) combining, to produce a combined sample, at least a portion of the one or more labeled standard samples with at least a portion of two or more differentially labeled samples, the differentially labeled samples being labeled with a different chemical moiety than the one or more labeled standard samples combined therewith; (f) loading at least a portion of the combined sample on a chromatographic column; (g) directing at least a portion of the eluent from the chromatographic column to a mass spectrometry system; (h) measuring the signature peptide-diagnostic daughter ion transition signal of one or more of the selected signature peptide-diagnostic daughter ion transitions; and (i) determining the absolute concentration of a protein of interest in one or more of the differentially labeled samples based at least on a comparison of the measured signature peptide-diagnostic daughter ion transition signal for the protein of interest to the measured signature peptide-diagnostic daughter ion transition signal for a labeled standard sample. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

In various embodiments, provided are methods for determining the concentration of one or more proteins of interest in two or more samples, comprising the steps of: (a) providing a standard sample for each of one or more proteins of interest, each standard sample comprising a signature peptide for the corresponding protein of interest; (b) selecting one or more signature peptide-diagnostic daughter ion transitions for at least one signature peptide of each standard sample; (c) generating a concentration curve for each selected diagnostic daughter ion; (d) labeling the one or more proteins of interest in two or more samples with different chemical moieties for each sample, the two or more samples thereby being differentially labeled; (e) labeling one or more standard samples with a chemical moiety; (f) combining, to produce a combined sample, at least a portion of the one or more labeled standard samples with at least a portion of two or more differentially labeled samples, the differentially labeled samples being labeled with a different chemical moiety than the one or more labeled standard samples combined therewith; (g) loading at least a portion of the combined sample on a chromatographic column; (h) directing at least a portion of the eluent from the chromatographic column to a mass spectrometry system; (i) measuring the signature peptide-diagnostic daughter ion transition signal of one or more of the selected signature peptide-diagnostic daughter ion transitions; and (j) determining the absolute concentration of a protein of interest in one or more of the labeled samples based at least on a comparison of the measured signature peptide-diagnostic daughter ion transition signal corresponding to the protein of interest to one or more of the concentration curve for that signature peptide-diagnostic daughter ion transition and the measured signature peptide-diagnostic daughter ion transition signal for a labeled standard sample. In various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization protein in the second sample; and normalizing the concentration ratio of the protein of interest using the concentration ratio of the normalization protein.

The standard samples comprising a signature peptide for the corresponding protein of interest (also referred to herein as “signature peptide standard samples”) are used, in various embodiments, to generate a concentration curve for each signature peptide and, in various embodiments, can act as an internal standard when measuring unknown samples. In various embodiments, the standard peptides can act as concentration normalizing standards when measuring unknown samples. In various embodiments, a standard sample comprises a signature peptide for a normalization protein.

In the present teachings a standard sample can be provided in a variety of ways. In various embodiments, a standard sample can be provided as a synthetic peptide, which is labeled and added in a known concentration to a sample under investigation to provide an internal standard. In various embodiments, a standard sample is provided from a control sample containing one or more proteins of interest. The control sample can be subjected to fragmentation (e.g., digestion) prior to or after labeling with a tag. The tag thus can be used to label one or more signature peptides in the one or more proteins of interest. The labeled control sample can be added to a sample under investigation to provide an internal standard. In various embodiments, the labeled control sample is added in a known concentration and can be used to determine absolute concentrations of one or more proteins of interest in the sample under investigation. In various embodiments, the labeled control sample is added at a fixed amount to a set of samples and can be used to determine the relative concentrations of one or more proteins of interest between the sets of samples under investigation.

A control sample can be provided in a variety of ways. For example, a control sample can comprise, for example, a normal sample, a pooled reference standard from all or some of the samples to be analyzed, or combinations thereof. For example, in various embodiments, a control sample comprises a normal patient sample that can serve as an internal standard to determine if samples under investigation differ from the normal sample, and thus, e.g., providing a potential indication of a disease state for a disease state. In various embodiments, the control sample is mixed into every sample to be analyzed at a substantially fixed ratio. In various embodiments, a fixed ratio of about 1:1 is used and, for example, can facilitate observation of both up-regulated and down-regulated peptides, proteins or both.

In various embodiments, the proteins of interest comprise cytochrome P450 isoforms, which include, but are not limited to, one or more of Cyp1a1, Cyp1a2, Cyp1b1, Cyp2a4, Cyp2a12, Cyp2b6, Cyp2b10, Cyp2c8, Cyp2c9, Cyp2c19, Cyp2c29/Cyp2c37, Cyp2c39, Cyp2c40, Cyp2d6, Cyp2d9, Cyp2d22/Cyp2d26, Cyp2e1, Cyp2f2, Cyp2j5, Cyp3a4, Cyp3a11, Cyp4a10/Cyp4a14, and combinations thereof. In various embodiments, the signature peptides comprise one or more of: CIGETIGR (SEQ. ID NO. 1), CIGEIPAK (SEQ. ID NO. 2); CIGEELSK (SEQ. ID NO. 3); YCFGEGLAR (SEQ. ID NO. 4); FCLGESLAK (SEQ. ID NO. 5); ICLGESIAR (SEQ. ID NO. 6); ICAGEGLAR (SEQ. ID NO. 7); VCAGEGLAR (SEQ. ID NO. 8); ICVGESLAR (SEQ. ID NO. 9); SCLGEALAR (SEQ. ID NO. 10); SCLGEPLAR (SEQ. ID NO. 11); VCVGEGLAR (SEQ. ID NO. 12); LCLGEPLAR (SEQ. ID NO. 13; ACLGEQLAK (SEQ. ID NO. 14); NCLGMR (SEQ. ID NO. 15); and NCIGK (SEQ. ID NO. 16); YIDLLPTSLPHAVTCDIK (SEQ. ID NO. 17); ICVGEGLAR (SEQ. ID NO. 18); ACLGEPLAR (SEQ. ID NO. 19); CIGEVLAK (SEQ. ID NO. 20); GFCMFDMECHK (SEQ. ID NO. 21); ICLGEGIAR (SEQ. ID NO. 22); LCQNEGCK (SEQ. ID NO. 23); GCPSLSELWR (SEQ. ID NO. 24); EECALEIIK (SEQ. ID NO. 25); GCPSLAEHWK (SEQ. ID NO. 26); VFANPEDCAFGK (SEQ. ID NO. 27).

In various embodiments, the present teachings facilitate identifying therapeutic candidate compounds, including antibodies and cellular immunotherapies. In various embodiments, the present teachings facilitate the study of drug metabolizing enzymes, (for example, cytochromes P450, uridine 5′-triphosophate glucuronosyltransferases, etc.). For example, the cytochrome P450 protein family of mono-oxygenases is responsible for the regulation of drug elimination in the liver and the formation of toxic drug metabolites. There are four major families of P450 isoforms with about 25 different isoforms, each with different substrate specificities inducible by different drugs or chemicals. This enzymatic behavior can make this family of proteins important in drug development. For example, the changes in expression of the different P450 proteins can provide information on the toxicity of different drugs and the possibility of forming dangerous drug metabolites. A system, method or assay to screen for multiple P450 isoforms could be of value in drug development, particularly if it yielded quantitative data relating to expression changes for individual isoforms.

In various aspects, provided are methods of assessing the response of a biological system to a chemical agent, comprising the steps of: (a) determining the absolute concentration of two or more proteins in a biological sample not exposed to a chemical agent; (b) determining the absolute concentration of two or more proteins in a biological sample exposed to the chemical agent; and (c) assessing the response of a biological system to the chemical agent based at least on the comparison of one or more of the absolute concentrations determined in step (a) to one or more of the absolute concentrations determined in step (b). In various embodiments, examples of biological systems (e.g., in vivo, in vitro, in silico, or combinations thereof) include, but are not limited to, whole organisms (e.g., a mammal, bacteria, virus, etc.), one or more sub-units of an whole organism (e.g., organ, tissue, cell, etc.), a biological or biochemical process, a disease state, a cell line, models thereof, and combinations thereof. In various embodiments, the chemical agent comprises one or more pharmaceutical agents, pharmaceutical compositions, or combinations thereof.

In various embodiments, the determination of absolute concentrations in the methods of assessing the response of a biological system to a chemical agent comprises one or more of the methods for determining the concentration of one or more proteins of interest in one or more samples described herein, one or more of the methods for determining the concentration of one or more proteins of interest in two or more samples described herein, or combinations thereof.

In various aspects, provided are assays designed to determine the level of expression of two or more proteins of interest in one or more samples. The assay can be, for example, an endpoint assay, a kinetic assay, or a combination thereof. The assay can, for example, be diagnostic of a disease or condition, prognostic of a disease or condition, or both. In various embodiments, provided are assays for determining the level of expression of two or more proteins in one or more samples using a method of the present teachings, comprises one or more of the methods for determining the concentration of one or more proteins of interest in one or more samples described herein, one or more of the methods for determining the concentration of one or more proteins of interest in two or more samples described herein, or combinations thereof.

In various aspects, provided are kits for performing a method, assay, or both of the present teachings. In various embodiments, a kit comprises two or more signature peptide standard samples, the signature peptides of two or more of the two or more signature peptide standard samples being signature peptides of different proteins. In various embodiments, a kit comprises five or more signature peptide standard samples, the signature peptides of ten or more of the five or more signature peptide standard samples being signature peptides of different cytochrome P450 isoforms. In various embodiments, a kit comprises ten or more signature peptide standard samples, the signature peptides of ten or more of the ten or more signature peptide standard samples being signature peptides of different cytochrome P450 isoforms.

In various embodiments, a kit comprises one or more signature peptide standard samples for one or more normalization proteins. For example, in various embodiments, a kit comprises one or more labeled signature peptide standard samples for normalization proteins where the signature peptides comprise one or more of: LCQNEGCK (SEQ. ID NO. 23); EECALEIIK (SEQ. ID NO. 25); GCPSLAEHWK (SEQ. ID NO. 26); and VFANPEDCAFGK (SEQ. ID NO. 27).

In various embodiments, a kit comprises signature peptide standard samples for signature peptides of one or more of the normalization proteins: corticosteroid 11-beta dehydrogenase isozyme 1, triglyceride transfer protein, and microsomal glutathione S-transferase.

In various embodiments, a kit for performing a method, assay, or both of the present teachings, on one or more samples derived from a mouse comprises signature peptide standard samples for signature peptides of one or more of the normalization proteins: corticosteroid 11-beta dehydrogenase isozyme 1, triglyceride transfer protein, microsomal glutathione S-transferase.

In various embodiments, a sample is derived from microsomal cells. Examples of suitable normalization proteins for microsomal cell derived samples include, but are not limited to: corticosteroid 11-beta dehydrogenase isozyme 1, triglyceride transfer protein, microsomal glutathione S-transferase, where, in various embodiments, the signature peptides are, respectively, LCQNEGCK (SEQ. ID NO. 23); EECALEIIK (SEQ. ID NO. 25); GCPSLAEHWK (SEQ. ID NO. 26); VFANPEDCAFGK (SEQ. ID NO. 27) (e.g., for mouse) or LCQNEGCK (SEQ. ID NO. 23); GCPSLSELWR (SEQ. ID NO. 24); EECALEIIK (SEQ. ID NO. 25); (e.g., for human) LCQNEGCK (SEQ. ID NO. 23); EECALEIIK (SEQ. ID NO. 25) (e.g., for mouse and human).

In various embodiments, a kit comprises signature peptide standard samples for signature peptides of the cytochrome P450 isoforms Cyp2a4, Cyp2a12, Cyp2b10, Cyp2c29/Cyp2c37, and Cyp2c40. In various embodiments, a kit comprises labeled signature peptide samples wherein the signature peptides comprise: YCFGEGLAR (SEQ. ID NO. 4); FCLGESLAK (SEQ. ID NO. 5); ICLGESIAR (SEQ. ID NO. 6); ICAGEGLAR (SEQ. ID NO. 7); and ICVGESLAR (SEQ. ID NO. 9). In various embodiments, a kit comprises signature peptide standard samples for signature peptides of one or more of the cytochrome P450 isoforms Cyp1a1, Cyp1a2, Cyp1b1, Cyp2a4, Cyp2a12, Cyp2b6, Cyp2b10, Cyp2c8, Cyp2c9, Cyp2c19, Cyp2c29/Cyp2c37, Cyp2c39, Cyp2c40, Cyp2d6, Cyp2d9, Cyp2d22/Cyp2d26, Cyp2e1, Cyp2f2, Cyp2j5, Cyp3a4, Cyp3a11, Cyp4a10/Cyp4a14, and combinations thereof. In various embodiments, the signature peptides comprise one or more of: CIGETIGR (SEQ. ID NO. 1), CIGEIPAK (SEQ. ID NO. 2); CIGEELSK (SEQ. ID NO. 3); YCFGEGLAR (SEQ. ID NO. 4); FCLGESLAK (SEQ. ID NO. 5); ICLGESIAR (SEQ. ID NO. 6); ICAGEGLAR (SEQ. ID NO. 7); VCAGEGLAR (SEQ. ID NO. 8); ICVGESLAR (SEQ. ID NO. 9); SCLGEALAR (SEQ. ID NO. 10); SCLGEPLAR (SEQ. ID NO. 11); VCVGEGLAR (SEQ. ID NO. 12); LCLGEPLAR (SEQ. ID NO. 13; ACLGEQLAK (SEQ. ID NO. 14); NCLGMR (SEQ. ID NO. 15); and NCIGK (SEQ. ID NO. 16); YIDLLPTSLPHAVTCDIK (SEQ. ID NO. 17); ICVGEGLAR (SEQ. ID NO. 18); ACLGEPLAR (SEQ. ID NO. 19); CIGEVLAK (SEQ. ID NO. 20); GFCMFDMECHK (SEQ. ID NO. 21); ICLGEGIAR (SEQ. ID NO. 22); LCQNEGCK (SEQ. ID NO. 23); GCPSLSELWR (SEQ. ID NO. 24); EECALEIIK (SEQ. ID NO. 25); GCPSLAEHWK (SEQ. ID NO. 26); VFANPEDCAFGK (SEQ. ID NO. 27) and combinations thereof.

As will be appreciated more fully from the following description in conjunction with the drawings, various embodiments of the present teachings can provide methods that facilitate the discovery, verification and/or validation of biomarkers; that facilitate the elucidation of basic biology and cell signaling; that facilitate drug discovery, or combinations thereof.

In various embodiments, the present teachings provide methods that facilitate the specific quantitation of a panel of proteins in a plasma, serum or other sample preparations. This quantitative assay can be used, for example, for the verification and/or validation of disease specific biomarkers, such as, e.g., cardiovascular disease biomarkers. In various embodiments, provided are methods for the quantitation of specific peptides for specific proteins using specific signature peptide-diagnostic daughter ion transitions.

In various embodiments the present teachings can elucidation of basic biology and cell signaling, for example, by facilitating the ability to quantitatively measure amount of a protein or proteins involved in a pathway; e.g., a labeled control standard being created from a “resting state” sample and being added into labeled perturbed state samples to facilitate quantitatively measuring changes in protein expression between resting and perturbed states.

In various embodiments the present teachings can facilitate drug discovery, for example, by facilitating the determination of the biological pathways effected by an agent. For example, various embodiments of the present teachings can be used to investigate a panel of proteins that represent good, or potential, drug targets. The method could be used to analyze samples that have been treated with a drug candidate to determine if any pathways have been affected, e.g., advantageous, negatively (e.g., toxic effect), or both. In various embodiments, a panel of proteins can be chosen to cover a broad spectrum of cellular pathways; and, for example, the qualitative and/or quantitative changes in protein expression used to obtain a greater understanding of the mode of action of the candidate therapeutic, the actual target, etc.

The foregoing and other aspects, embodiments, and features of the teachings can be more fully understood from the following description in conjunction with the accompanying drawings. In the drawings like reference characters generally refer to like features and structural elements throughout the various figures. The drawings are not necessarily to scale, emphasis instead being placed upon illustrating the principles of the teachings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B are a schematic diagram of various embodiments of methods of determining the absolute concentration of a protein in a sample.

FIG. 2 is a simplified schematic diagram of the mass spectrometer system used in Examples 1 and 2.

FIG. 3 is a MRM chromatogram of 3.2 finol on column of each labeled synthetic signature peptide of Examples 1 and 2.

FIG. 4 is a concentration curve generated for the diagnostic daughter ion of the ICLGESIAR peptide (the signature peptide chosen for the Cyp2b10 isoform of P450) of Examples 1 and 2.

FIG. 5 is a MRM chromatogram for the diagnostic daughter ion of the ICLGESIAR peptide (the signature peptide chosen for the Cyp2b10 isoform of P450) of Example 1, for both control and phenobarbital induced samples.

FIG. 6 shows MRM scan data for the quantitation of P450 proteins within the same subfamily.

FIG. 7 illustrates the results of a Western blot analysis of four of the subfamilies of P450 proteins: Cyp1a1, Cyp1a2, Cyp2e1 and Cyp3a4.

FIG. 8 illustrates a work flow used in Example 3.

FIG. 9 depicts data on the reproducibility of the measurements of Example 3.

FIG. 10 illustrates a pooled reference sample workflow for Example 3.

FIG. 11 illustrates a workflow used in Example 4 when using mTRAQ™ brand reagents.

FIGS. 12A-B depict MRM triggered MS/MS data on a peptide of filamin A in Example 4 that can be used, for example, to develop MRM assays for this peptide and confirm the identity of the signature peptide.

FIGS. 13A-B depict MRM triggered MS/MS data on a peptide of laminin alpha 5 in Example 4 that can be used, for example, to develop MRM assays for this peptide and confirm the identity of the signature peptide.

FIGS. 14A-E compare total ion current data for a fixed MRM transition for a peptide of filamin A protein in Example 4.

FIGS. 15A-E compare total ion current data for a fixed MRM transition for a peptide of laminin alpha 5 protein in Example 4.

### DETAILED DESCRIPTION OF VARIOUS EMBODIMENTS

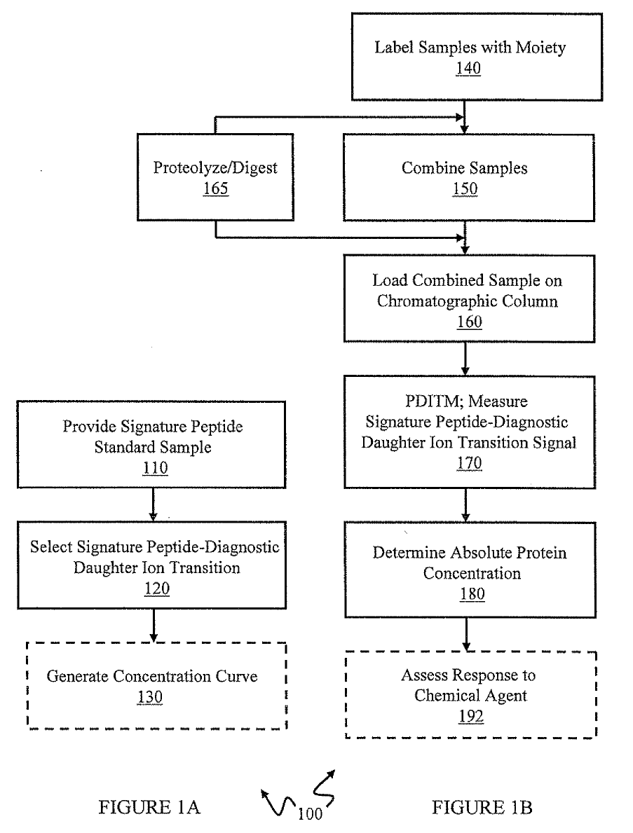

Referring to FIGS. 1A and 1B, in various embodiments, methods for determining the absolute concentration of a protein in a sample provide a signature peptide standard sample (step 110) for each protein of interest in one or more samples. For example, for each individual protein isoform of interest, a peptide substantially unique to the individual isoform is selected as a signature peptide for that isoform. In various embodiments, more than one signature peptide can be selected for a given isoform and a signature peptide standard sample can be prepared for each of the selected signature peptides of that isoform (e.g., the use of multiple signature peptides for a single protein can provide cross-verification of the concentrations determined using the different signature peptide standard samples for that protein). The signature peptide standard samples can be derived, for example, from proteins that are known and/or anticipated to be unchanged by the conditions of the experiment. For example, the signature peptide standard can be derived from a control sample containing one or more of the proteins of interest, such as, e.g., a normal patient sample, a known concentration sample, etc. The signature peptide standard samples can be unlabeled or labeled with a chemical moiety.

A sample of the signature peptide for each isoform of interest can be prepared synthetically and labeled with a chemical moiety. A sample of the signature peptide for each isoform can be prepared by labeling with a chemical moiety non-synthetic isoforms in one or more samples prior to or after digestion of the isoforms in the one or more samples. Examples of chemical moieties suitable for labeling include, but are not limited to, labeling with an isotope coded affinity tag (e.g., an ICAT® brand reagent), with an isobaric (same mass) tag (e.g. iTRAQ™ reagent), a mass differential tag (e.g., a mTRAQ™ brand reagent) etc.; and the concentration of the signature peptide in each labeled signature peptide sample can be determined using, for example, amino acid analysis (AAA) on a portion of the sample.

In various embodiments, the signature peptide standard sample is cleaned up (e.g., to remove, e.g., interfering sample, buffer artifacts, etc; by, e.g., high performance liquid chromatography (HPLC), reverse phase (RP)-HPLC, exchange fractionation, etc., and combinations thereof) before the concentration of the signature peptide in the labeled signature peptide sample is determined. In various embodiments, the signature peptide standard sample is labeled with substantially the same chemical moiety as applied to one or more of the samples to be analyzed. In various embodiments, the signature peptide standard sample is labeled with a different chemical moiety as applied to one or more of the samples (such as, e.g., when a signature peptide standard sample is used an internal standard). For example, in various embodiments, a standard sample comprises a signature peptide for a normalization protein.

At least a portion of a signature peptide standard sample can be subjected to PDITM scans (e.g. MRM scans) to select one or more diagnostic daughter ions for that signature peptide (step 120) and thereby select a signature peptide-daughter ion transition for the signature peptide of the standard sample. It is to be understood that same diagnostic daughter ion (e.g., having the same mass, the same structure, etc.) can be selected for different signature peptides. In various embodiments, the signature peptide standard sample is cleaned up (e.g., to remove, e.g., interfering sample, buffer artifacts, etc; by, e.g., high performance liquid chromatography (HPLC), reverse phase (RP)-HPLC, exchange fractionation, etc., and combinations thereof) before it is used to select a diagnostic daughter ion. Diagnostic daughter ions for a signature peptide can be selected, for example, based on one or more of their: level of detection (LOD), limit of quantitation (LOQ), signal-to-noise (S/N) ratio, mass similarity with other daughter ions of other signature peptides, and linearity of quantitation over a specific dynamic range of concentrations. In various embodiments, the dynamic range of concentrations of interest is about three to about four orders of magnitude depending, for example, on the mass analyzer system being used. In various embodiments, the LOQ ranges from about attomole levels (10-18 moles) to about femtomole levels (10-15 moles) per microgram (μg) of sample, with a dynamic range of about three to about four orders of magnitude above the LOQ.

The same signature peptide standard sample portion used to select a diagnostic daughter ion or another portion of a signature peptide standard sample can be used to determine parent-daughter ion transition monitoring conditions for the mass analyzer system. For example, where the mass analyzer system comprises a liquid chromatography (LC) component, the signature peptide standard sample can be used to determine chromatography retention times. In various embodiments, the signature peptide standard sample can be used to determine for the signature peptide in the sample its ionization efficiency in the ion source and fragmentation efficiency in the ion fragmentor under various conditions.

Referring again to FIGS. 1A and 1B, in various embodiments, the same portion used to select a diagnostic daughter ion or another portion of a signature peptide standard sample is subject to PDITM to generate one or more concentrations curves for the selected signature peptide-diagnostic daughter ion transition (step 130) based on the ion signal for the corresponding diagnostic daughter ion. The ion signal for the diagnostic daughter ion can, for example, be based on the intensity (average, mean, maximum, etc.) of the diagnostic daughter ion peak, the area of the diagnostic daughter ion peak, or a combination thereof. In various embodiments, the generation of a concentration curve can use one or more internal standards included in at least a portion of the signature peptide standard sample to, e.g., facilitate concentration determinations, account for differences in injection volume, etc.

In various embodiments, a concentration curve can be generated by using PDITM to measure the ion signal of a diagnostic daughter ion associated with the corresponding signature peptide standard sample; and generating a concentration curve by linear extrapolation of the measured concentration such that zero concentration corresponds to zero diagnostic daughter ion signal. In various embodiments, a concentration curve can be generated by using PDITM to measure the ion signal of a diagnostic daughter ion associated with the corresponding signature peptide standard sample at two or more known concentrations; and generating a concentration curve by fitting a function to the measured diagnostic daughter ion signals. Suitable fitting functions can depend, for example, on the response of the detector (e.g., detector saturation, non-linearity, etc.). In various embodiments, the fitting function is a linear function.

In various embodiments, sample preparation and signature peptide standard sample preparation label proteins, peptides, or both, with a chemical moiety (e.g., tag). A wide variety of chemical moieties and labeling approaches can be used in the present teachings. For example, differentially isotopically labeled protein reactive reagents, as described in published PCT patent application WO 00/11208, the entire contents of which are incorporated herein by reference, can be used to label one or more signature peptides with a chemical moiety. In various embodiments, mass differential reagents, such as, for example, the mTRAQ brand reagent method can be used. In various embodiments, labeling of proteins with isotopically coded affinity reagents such as, for example, the ICAT® brand reagent method can be used. In various embodiments, isobaric reagents (reagents which provide labels which are of the same mass but which produce different signals following labeled parent ion fragmentation, e.g., by collision induced dissociation (CID) such as, for example, the iTRAQ™ brand reagent method) can be used. In various embodiments, a set of isobaric (same mass) reagents which yield amine-derivatized peptides that are chromatographically identical and indistinguishable in MS, but which produce strong low-mass MS/MS signature ions following CID can be used. In various embodiments, an affinity separation can be performed on one or more proteins, peptides, or both, of one or more samples before, after, or both before and after, labeling with one or more isobaric reagents.

In various embodiments, the isotope coded affinity labeled protein reactive reagents have three portions: an affinity label (A) covalently linked to a protein reactive group (PRG) through a cleavable linker group (L) that includes an isotopically labeled linker. The linker can be directly bonded to the protein reactive group (PRG). The affinity labeled protein reactive reagents can have the formula:

A-L-PRG

The linker can be differentially isotopically labeled, e.g., by substitution of one or more atoms in the linker with a stable isotope thereof. For example, hydrogens can be substituted with deuteriums (2H) and/or ¹²C substituted with ¹³C. Utilization of ¹³C promotes co-elution of the heavy and light isotopes in reversed phase chromatography.

The affinity label (A) can function as a means for separating reacted protein (labeled with a PRG) from unreacted protein (not labeled with a PRG) in a sample. In various embodiments, the affinity label comprises biotin. After reaction of the PRG portion of the reagent with protein, affinity chromatography can be used to separate labeled and unlabeled components of the sample. Affinity chromatography can be used to separate labeled and unlabeled proteins, labeled and unlabeled digestion products of the proteins (i.e., peptides) or both. Thereafter, the cleavage of the cleavable linker (L) can be effected such as, for example, chemically, enzymatically, thermally or photochemically to release the isolated materials for mass spectrometric analysis. In various embodiments, the linker can be acid-cleavable.

In various embodiments the PRG can be incorporated on a solid support (S) as shown in the following formula:

S-L-PRG

The solid support can be composed of, for example, polystyrene or glass, to which cleavable linker and protein reactive groups are attached. The solid support can be used as a means of peptide separation and sample enrichment (e.g., as chromatography media in the form of a column). Unlabeled digestion products, for example, can be linked to the modified solid support via the PRG, labeled and then released by various means (e.g. chemical or enzymatic) from the solid support.

Prior to mass spectrometric analysis, the bound protein can be digested to form peptides including bound peptides which can be analyzed by mass spectrometry. The protein digestion step can precede or follow cleavage of the cleavable linker. In some embodiments, a digestion step (e.g., enzymatic cleavage) may not be necessary, where, for example, the proteins are relatively small. In various embodiments, the insertion of an acid cleavable linker can result in a smaller and more stable label. A smaller and more stable linker can afford enhanced ion fragmentation, e.g., in CID.

Examples of PRG groups include, but are not limited to: (a) those groups that selectively react with a protein functional group to form a covalent or non-covalent bond tagging the protein at specific sites, and (b) those that are transformed by action of the protein, e.g., that are substrates for an enzyme. In various embodiments, a PRG can be a group having specific reactivity for certain protein groups, such as specificity for sulfhydryl groups. Such a PRG can be useful, for example, in general for selectively tagging proteins in complex mixtures. For example, a sulfhydryl specific reagent tags proteins containing cysteine.

In various embodiments, a PRG group that selectively reacts with certain groups that are typically found in peptides (e.g., sulfhydryl, amino, carboxy, hydroxy, lactone groups) can be introduced into a mixture containing proteins. In various embodiments, after reaction with the PRG, proteins in the complex mixture are cleaved, e.g., enzymatically, into a number of peptides.

Referring again to FIGS. 1A and 1B, the determination of the absolute concentration of one or more proteins in one or more samples proceeds with labeling one or more of the proteins in one or more of the samples (step 140) with a chemical moiety. In various embodiments, this step of labeling comprises differentially labeling one or more proteins in two or more samples, where different chemical moieties are used to label proteins in different samples. A wide variety of chemical moieties can be used to perform the labeling, differential labeling, or both, including, but not limited to, those described above and elsewhere herein. For example, isotopically different labels, different isobaric reagents, or combinations thereof can be used to differentially label samples. A wide variety of samples can be used including, but not limited to, biological fluids, and cell or tissue lysates. The samples can be from different sources or conditions, for example, control vs. experimental, samples from different points in time (e.g., to form a sequence), disease vs. normal, experimental vs. disease, etc.

In various embodiments, differential labeling is used for multiplexing, so that within one experimental run, for example, multiple different isoforms from different samples (e.g., control, treated) can be compared; multiple mutant strains can be compared with a wild type; in a time course scenario, multiple dosage levels can be assessed against a baseline; different isolates of cancer tissue can be evaluated against normal tissue; or combinations thereof in a single run. In various embodiments, differential labeling on subclasses of peptides (e.g. phosphorylation), can be used to uncover post-translational modifications (PTM's).

In various embodiments, at least a portion of the labeled samples, labeled signature peptide standard samples, or both, are then combined (step 150) and at least a portion of the combined sample is loaded on a chromatographic column (step 160) (e.g., a LC column, a gas chromatography (GC) column, or combinations thereof). In various embodiments, labeled samples, labeled signature peptide standard samples, or both, are combined (step 150) according to one or more of the following to produce a combined sample:

(i) a labeled sample (e.g., a control sample, an experimental sample) is combined with one or more signature peptide standard samples (the signature peptides of the standard samples corresponding to the signature peptides of one or more proteins of interest);

(ii) a labeled sample (e.g., a control sample, an experimental sample) is combined with one or more labeled signature peptide standard samples, the signature peptides of the standard samples corresponding to the signature peptides of one or more proteins of interest and the labeled signature peptide samples being differentially labeled with respect to the labeled sample;

(iii) two or more differentially labeled samples (e.g., control and experimental; experimental #1 and experimental #2; multiple controls and multiple experimental samples; etc) are combined;

(iv) two or more differentially labeled samples are combined with one or more signature peptide standard samples;

(v) two or more differentially labeled samples are combined with one or more labeled signature peptide standard samples, the labeled signature peptide standard samples being differentially labeled with respect to the differentially labeled samples; and/or

(vi) combinations thereof.

For example, the addition of a signature peptide standard sample can serve as an internal standard for the corresponding signature peptide. In various embodiments, a signature peptide standard sample comprises a signature peptide for a normalization protein. A signature peptide standard sample combined with a sample can be referred to as a “signature peptide internal standard sample”. Accordingly, in various embodiments, a signature peptide standard sample for each protein of interest in a sample is combined with the sample prior to loading on the chromatographic column. In various embodiments, the different samples are combined in substantially equal amounts.

For example, in various embodiments, a control standard can be provided that is labeled with one reagent from a label from a set of labeling reagents (e.g., iCAT brand reagents, iTRAQ brand reagents, mTRAQ brand reagents, etc.) to produce a labeled signature peptide standard sample. It is to be understood that the labeled signature peptides of this standard may still be part of a larger protein until subjected to, for example, digestion. This labeled control standard can be added into each of the labeled samples to be analyzed to produce a combined sample, and. The labeled samples being labeled with a different label than the label used in producing the labeled control standard.

A protein digestion step (step 165) can precede, follow, or both proceed and follow the step of combining (step 150). In various embodiments, proteins in a sample, the combined sample, or both are enzymatically digested (proteolyzed), to generate peptides (step 165). In some embodiments, a digestion step (e.g., enzymatic cleavage) may not be necessary, where, for example, the proteins are relatively small.

At least a portion of the eluent from the chromatographic column is then directed to a mass spectrometry system and the signature peptide-diagnostic daughter ion transition signal of one or more selected signature peptide-diagnostic daughter ion transitions is measured (step 170) using PDITM (e.g., MRM). The mass analyzer system comprises a first mass separator, and ion fragmentor and a second mass separator. The transmitted parent ion m/z range of a PDITM scan (selected by the first mass separator) is selected to include a m/z value of one or more of the signature peptides and the transmitted daughter ion m/z range of a PDITM scan (selected by the second mass separator) is selected to include a m/z value one or more of the selected diagnostic daughter ions corresponding to the transmitted signature peptide.

The absolute concentration of a protein of interest in a sample is then determined (step 180). In various embodiments, the absolute concentration of a protein of interest is determined by comparing the measured ion signal of the corresponding signature peptide-diagnostic daughter ion transition (the signature peptide-diagnostic daughter ion transition signal) to one or more of:

(i) the concentration curve for that signature peptide-diagnostic daughter ion transition;

(ii) the signature peptide-diagnostic daughter ion transition signal for a signature peptide internal standard sample;

(iii) the concentration curve for that signature peptide-diagnostic daughter ion transition and the signature peptide-diagnostic daughter ion transition signal for a signature peptide internal standard sample; and/or

(iv) combinations thereof.

In various embodiments, one or more proteins of interest can be used for, e.g., normalization of diagnostic daughter ion signals, normalization of the concentration of a protein in a first sample relative the concentration in a second sample (e.g., normalize a concentration ratio), evaluation of data reliability, evaluation of starting sample amount across samples, or combinations thereof. Accordingly, in various embodiments, one or more proteins of interest are normalization proteins which, e.g., are anticipated to have substantially the same concentration in two or more of the two or more samples, are anticipated to have a concentration that is not substantially affected by treatment of a sample with a chemical agent, or both. For example, in various embodiments, a protein of interest can be a protein known to have substantially the same concentration between samples.

In various embodiments, changes in the signal level of a signature peptide of a normalization protein can be used to normalize the signal levels of the signature peptides of one or more proteins of interest. In various embodiments, the relative signal level of a signature peptide of a normalization protein between two samples is used to normalize the relative concentration of a protein of interest between two samples. For example, in various embodiments, the methods comprise a step of assessing the response of a biological system to a chemical agent, assessing the disease state of a biological system, or both, based at least on a comparison of the absolute concentrations of two or more proteins in one or more of the two or more samples. In various embodiments, the step of assessing comprises determining a concentration ratio between two samples for a protein of interest by comparing the concentration of a protein of interest in a first sample relative to the concentration of said protein of interest in a second sample, determining a concentration ratio between two samples for a normalization protein by comparing the concentration of normalization protein in the first sample relative to the concentration of said normalization in the second sample; and normalizing the concentration ratio of the protein of interest using the concentration ratio of the normalization protein. For example, in various embodiments where the ratio of the normalization signature peptide signal between two samples (e.g., control vs. experimental, samples from different points in time (e.g., to form a sequence), disease vs. normal, experimental vs. disease, etc.) varies from 1:1, such a variation can be indicative of, e.g., differences in starting amounts between the two sample, sample handling error, or other systematic or random errors. In various embodiments, the ratio of the normalization signature peptide signal between two samples is used to normalize the concentration ratio of a protein of interest for these two samples. In various embodiments, the ratio for the normalization protein is used as a median ratio and the concentration ratios of one or more proteins of interest are corrected to this median.

In various embodiments, differences in the signature peptide signal level of a normalization protein between two samples can be used to evaluate data reliability. For example, where the signature peptide signal associated with a normalization protein varies by a significant amount between samples, the data associated with one or both of these samples is excluded as unreliable. In various embodiments, variations by more than about one standard deviation are considered significant. In various embodiments, variations by more than about two standard deviations are considered significant. In various embodiments, where the ratio of the normalization signature peptide signal between two samples differs significantly from 1:1 the data associated with one or both of these samples is considered unreliable. In various embodiments, where the diagnostic daughter ion signal of the normalization protein in one sample varies by more than about ±10% relative to the diagnostic daughter ion signal in another sample, such variation is considered significant. In various embodiments, where the diagnostic daughter ion signal of the normalization protein in one sample varies by more than about ±20% relative to the diagnostic daughter ion signal in another sample, such variation is considered significant. In various embodiments, where the diagnostic daughter ion signal of the normalization protein in one sample varies by more than about ±50% relative to the diagnostic daughter ion signal in another sample, such variation is considered significant.

In various embodiments, the standard sample comprises a labeled pooled reference standard. A pooled reference sample can be created in a variety of ways, for example, a pooled reference sample can be provided from a number of patient samples sharing a common feature (all substantially lacking a certain disease state, all possessing a certain disease state, all under a certain age, etc.); a portion of one or more of the samples under investigation, and combinations thereof. Accordingly, in various embodiments, a pooled reference sample is substantially similar in its components to the sample of interests. For example, where a pooled reference sample is provided by combining a portion of each of the samples under investigation, every peptide in the labeled samples of interest has a corresponding labeled peptide in the labeled standard sample.

In various embodiments, the measured ion signal for the selected diagnostic daughter ion corresponding to the protein of interest from a labeled pooled reference sample can be used to compare relative changes in peptide/protein concentration across many samples which have had the same pooled reference standard added in at equivalent ratios. Accordingly, in various embodiments, a pooled reference sample can be used as a normalization sample. It is to be understood, this comparison might not reflect the absolute amount of protein present but can be used to determine the relative differences between the samples of that protein analyzed on different instruments, under different conditions, etc.

Generally in the present teachings, it is not necessary to determine the absolute concentration of a normalization protein because, e.g., the ratio of the signature peptide signal associated with a normalization protein in one sample to that in another sample can be used to normalize the signal levels of the signature peptides of one or more proteins of interest, normalization of diagnostic daughter ion signals, normalization of the concentration of a protein in a first sample relative the concentration in a second sample (e.g., normalize a concentration ratio), evaluate the reliability of data, evaluation of starting sample amount across samples, or combinations thereof.

In various embodiments, the absolute concentration determinations can be used to understand the basal expression levels of proteins of interest in wild-type or control sample or populations of samples. In various embodiments, the absolute concentration determinations can be applied to screen for and identify proteins which exhibit differential expression in cells, tissue or biological fluids. In various embodiments, the absolute concentration determinations can be used to assess the response of a biological system to a chemical agent (step 192). For example, the absolute concentrations can be used to determine the response of a patient, or a model (e.g., animal, disease, cell, biochemical, etc.) to treatment by a pharmaceutical agent or pharmaceutical composition, exposure to an organism (e.g., virus, bacteria), an environmental contaminant (e.g., toxin, pollutant), etc.

A wide variety of mass analyzer systems can be used in the present teachings to perform PDITM. Suitable mass analyzer systems include two mass separators with an ion fragmentor disposed in the ion flight path between the two mass separators. Examples of suitable mass separators include, but are not limited to, quadrupoles, RF muiltipoles, ion traps, time-of-flight (TOF), and TOF in conjunction with a timed ion selector. Suitable ion fragmentors include, but are not limited to, those operating on the principles of: collision induced dissociation (CID, also referred to as collisionally assisted dissociation (CAD)), photoinduced dissociation (PID), surface induced dissociation (SID), post source decay, or combinations thereof.

Examples of suitable mass spectrometry systems for the mass analyzer include, but are not limited to, those which comprise a triple quadrupole, a quadrupole-linear ion trap, a quadrupole TOF systems, and TOF-TOF systems.

Suitable ion sources for the mass spectrometry systems include, but are not limited to, an electrospray ionization (ESI), matrix-assisted laser desorption ionization (MALDI), atmospheric pressure chemical ionization (APCI), and atmospheric pressure photoionization (APPI) sources. For example, ESI ion sources can serve as a means for introducing an ionized sample that originates from a LC column into a mass separator apparatus. One of several desirable features of ESI is that fractions from the chromatography column can proceed directly from the column to the ESI ion source.

In various embodiments, the mass spectrometer system comprises a triple quadrupole mass spectrometer for selecting a parent ion and detecting fragment daughter ions thereof. In various embodiments, the first quadrupole selects the parent ion. The second quadrupole is maintained at a sufficiently high pressure and voltage so that multiple low energy collisions occur causing some of the parent ions to fragment. The third quadrupole is selected to transmit the selected daughter ion to a detector. In various embodiments, a triple quadrupole mass spectrometer can include an ion trap disposed between the ion source and the triple quadrupoles. The ion trap can be set to collect ions (e.g., all ions, ions with specific m/z ranges, etc.) and after a fill time, transmit the selected ions to the first quadrupole by pulsing an end electrode to permit the selected ions to exit the ion trap. Desired fill times can be determined, e.g., based on the number of ions, charge density within the ion trap, the time between elution of different signature peptides, duty cycle, decay rates of excited state species or multiply charged ions, or combinations thereof.

In various embodiments, one or more of the quadrupoles in a triple quadrupole mass spectrometer can be configurable as a linear ion trap (e.g., by the addition of end electrodes to provide a substantially elongate cylindrical trapping volume within the quadrupole). In various embodiments, the first quadrupole selects the parent ion. The second quadrupole is maintained at a sufficiently high collision gas pressure and voltage so that multiple low energy collisions occur causing some of the parent ions to fragment. The third quadrupole is selected to trap fragment ions and, after a fill time, transmit the selected daughter ion to a detector by pulsing an end electrode to permit the selected daughter ion to exit the ion trap. Desired fill times can be determined, e.g., based on the number of fragment ions, charge density within the ion trap, the time between elution of different signature peptides, duty cycle, decay rates of excited state species or multiply charged ions, or combinations thereof.

In various embodiments, the mass spectrometer system comprises two quadrupole mass separators and a TOF mass spectrometer for selecting a parent ion and detecting fragment daughter ions thereof. In various embodiments, the first quadrupole selects the parent ion. The second quadrupole is maintained at a sufficiently high pressure and voltage so that multiple low energy collisions occur causing some of the ions to fragment, and the TOF mass spectrometer selects the daughter ions for detection, e.g., by monitoring the ions across a mass range which encompasses the daughter ions of interest and extracted ion chromatograms generated, by deflecting ions that appear outside of the time window of the selected daughter ions away from the detector, by time gating the detector to the arrival time window of the selected daughter ions, or combinations thereof.

In various embodiments, the mass spectrometer system comprises two TOF mass analyzers and an ion fragmentor (such as, for example, CID or SID). In various embodiments, the first TOF selects the parent ion (e.g., by deflecting ions that appear outside the time window of the selected parent ions away from the fragmentor) for introduction in the ion fragmentor and the second TOF mass spectrometer selects the daughter ions for detection, e.g., by monitoring the ions across a mass range which encompasses the daughter ions of interest and extracted ion chromatograms generated, by deflecting ions that appear outside of the time window of the selected daughter ions away from the detector, by time gating the detector to the arrival time window of the selected daughter ions, or combinations thereof. The TOF analyzers can be linear or reflecting analyzers.

In various embodiments, the mass spectrometer system comprises a time-of-flight mass spectrometer and an ion reflector. The ion reflector is positioned at the end of a field-free drift region of the TOF and is used to compensate for the effects of the initial kinetic energy distribution by modifying the flight path of the ions. In various embodiments ion reflector consists of a series of rings biased with potentials that increase to a level slightly greater than an accelerating voltage. In operation, as the ions penetrate the reflector they are decelerated until their velocity in the direction of the field becomes zero. At the zero velocity point, the ions reverse direction and are accelerated back through the reflector. The ions exit the reflector with energies identical to their incoming energy but with velocities in the opposite direction. Ions with larger energies penetrate the reflector more deeply and consequently will remain in the reflector for a longer time. The potentials used in the reflector are selected to modify the flight paths of the ions such that ions of like mass and charge arrive at a detector at substantially the same time.

In various embodiments, the mass spectrometer system comprises a tandem MS-MS instrument comprising a first field-free drift region having a timed ion selector to select a parent ion of interest, a fragmentation chamber (or ion fragmentor) to produce daughter ions, and a mass separator to transmit selected daughter ions for detection. In various embodiments, the timed ion selector comprises a pulsed ion deflector. In various embodiments, the ion deflector can be used as a pulsed ion deflector. The mass separator can include an ion reflector. In various embodiments, the fragmentation chamber is a collision cell designed to cause fragmentation of ions and to delay extraction. In various embodiments, the fragmentation chamber can also serve as a delayed extraction ion source for the analysis of the fragment ions by time-of-flight mass spectrometry.

In various embodiments, the mass spectrometer system comprises a tandem TOF-MS having a first, a second, and a third TOF mass separator positioned along a path of the plurality of ions generated by the pulsed ion source. The first mass separator is positioned to receive the plurality of ions generated by the pulsed ion source. The first mass separator accelerates the plurality of ions generated by the pulsed ion source, separates the plurality of ions according to their mass-to-charge ratio, and selects a first group of ions based on their mass-to-charge ratio from the plurality of ions. The first mass separator also fragments at least a portion of the first group of ions. The second mass separator is positioned to receive the first group of ions and fragments thereof generated by the first mass separator. The second mass separator accelerates the first group of ions and fragments thereof, separates the first group of ions and fragments thereof according to their mass-to-charge ratio, and selects from the first group of ions and fragments thereof a second group of ions based on their mass-to-charge ratio. The second mass separator also fragments at least a portion of the second group of ions. The first and/or the second mass separator may also include an ion guide, an ion-focusing element, and/or an ion-steering element. In various embodiments, the second TOF mass separator decelerates the first group of ions and fragments thereof. In various embodiments, the second TOF mass separator includes a field-free region and an ion selector that selects ions having a mass-to-charge ratio that is substantially within a second predetermined range. In various embodiments, at least one of the first and the second TOF mass separator includes a timed-ion-selector that selects fragmented ions. In various embodiments, at least one of the first and the second mass separators includes an ion fragmentor. The third mass separator is positioned to receive the second group of ions and fragments thereof generated by the second mass separator. The third mass separator accelerates the second group of ions and fragments thereof and separates the second group of ions and fragments thereof according to their mass-to-charge ratio. In various embodiments, the third mass separator accelerates the second group of ions and fragments thereof using pulsed acceleration. In various embodiments, an ion detector positioned to receive the second group of ions and fragments thereof. In various embodiments, an ion reflector is positioned in a field-free region to correct the energy of at least one of the first or second group of ions and fragments thereof before they reach the ion detector.

In various embodiments, the mass spectrometer system comprises a TOF mass analyzer having multiple flight paths, multiple modes of operation that can be performed simultaneously in time, or both. This TOF mass analyzer includes a path selecting ion deflector that directs ions selected from a packet of sample ions entering the mass analyzer along either a first ion path, a second ion path, or a third ion path. In some embodiments, even more ion paths may be employed. In various embodiments, the second ion deflector can be used as a path selecting ion deflector. A time-dependent voltage is applied to the path selecting ion deflector to select among the available ion paths and to allow ions having a mass-to-charge ratio within a predetermined mass-to-charge ratio range to propagate along a selected ion path.

For example, in various embodiments of operation of a TOF mass analyzer having multiple flight paths, a first predetermined voltage is applied to the path selecting ion deflector for a first predetermined time interval that corresponds to a first predetermined mass-to-charge ratio range, thereby causing ions within first mass-to-charge ratio range to propagate along the first ion path. In various embodiments, this first predetermined voltage is zero allowing the ions to continue to propagate along the initial path. A second predetermined voltage is applied to the path selecting ion deflector for a second predetermined time range corresponding to a second predetermined mass-to-charge ratio range thereby causing ions within the second mass-to-charge ratio range to propagate along the second ion path. Additional time ranges and voltages including a third, fourth etc. can be employed to accommodate as many ion paths as are required for a particular measurement. The amplitude and polarity of the first predetermined voltage is chosen to deflect ions into the first ion path, and the amplitude and polarity of the second predetermined voltage is chosen to deflect ions into the second ion path. The first time interval is chosen to correspond to the time during which ions within the first predetermined mass-to-charge ratio range are propagating through the path selecting ion deflector and the second time interval is chosen to correspond to the time during which ions within the second predetermined mass-to-charge ratio range are propagating through the path selecting ion deflector. A first TOF mass separator is positioned to receive the packet of ions within the first mass-to-charge ratio range propagating along the first ion path. The first TOF mass separator separates ions within the first mass-to-charge ratio range according to their masses. A first detector is positioned to receive the first group of ions that are propagating along the first ion path. A second TOF mass separator is positioned to receive the portion of the packet of ions propagating along the second ion path. The second TOF mass separator separates ions within the second mass-to-charge ratio range according to their masses. A second detector is positioned to receive the second group of ions that are propagating along the second ion path. In some embodiments, additional mass separators and detectors including a third, fourth, etc. may be positioned to receive ions directed along the corresponding path. In one embodiment, a third ion path is employed that discards ions within the third predetermined mass range. The first and second mass separators can be any type of mass separator. For example, at least one of the first and the second mass separator can include a field-free drift region, an ion accelerator, an ion fragmentor, or a timed ion selector. The first and second mass separators can also include multiple mass separation devices. In various embodiments, an ion reflector is included and positioned to receive the first group of ions, whereby the ion reflector improves the resolving power of the TOF mass analyzer for the first group of ions. In various embodiments, an ion reflector is included and positioned to receive the second group of ions, whereby the ion reflector improves the resolving power of the TOF mass analyzer for the second group of ions.

The following example illustrates experiments in which the absolute concentrations of multiple isoforms of cytochrome P450 in two different samples were determined in a multiplex manner. The teachings of this example are not exhaustive, and are not intended to limit the scope of these experiments or the present teachings.

### Example 1

### P450 Isoforms

In this example, absolute quantitation of a set of sixteen P450 isoforms is shown. This example can provide, for example, an assay for multiple P450 isoforms conductible in a single experimental run. Peptides specific to individual P450 isoforms were synthesized, labeled with a stable isotope tag (light Cleavable ICAT Reagent) and purified by HPLC to provide labeled signature peptide standard samples. These standard peptide samples were used to create a concentration curve using quantitative Multiple Reaction Monitoring (MRM) scans. Mouse liver microsome samples, control (CT) and phenobarbital induced (IND) were then labeled with heavy cleavable ICAT reagents. Phenobarbital (PB) is often used as a representative chemical for industrial solvents, pesticides, etc and is known to induce several P450 genes in subfamilies 2a, 2b, 2c and 3a. Control and Induced samples were loaded separately on the chromatographic column. Prior to loading on the chromatographic column, the control and induced samples were combined with a signature peptide internal standard sample for each signature peptide (labeled with a light cleavable ICAT reagent). Comparison of the chromatographic areas of the light (internal standard) and heavy peptide (sample) in a combined sample to the concentration curve provided quantitative information on the level of each P450 investigated in the control sample and the change in expression upon treatment with phenobarbital. Sixteen different labeled synthetic peptides, representing 16 different P450 proteins, were monitored in this experiment. The sixteen P450 proteins studied in this example are listed in column 1 of Table 1.

TABLE 1

| | | |
|---|---|---|
| Protein | Signature Peptide | MRM |

| | | |
|---|---|---|
| Cyp1a1 | CIGETIGR | 538.3/632.3 |
| | (SEQ. ID NO. 1) | |
| Cyp1a2 | CIGEIPAK | 529.3/315.3 |
| | (SEQ. ID NO. 2) | |
| Cyp1b1 | CIGEELSK | 553.3/662.3 |
| | (SEQ. ID NO. 3) | |
| Cyp2a4 | YCFGEGLAR | 621.8/749.4 |
| | (SEQ. ID NO. 4) | |
| Cyp2a12 | FCLGESLAK | 590.8/703.4 |
| | (SEQ. ID NO. 5) | |
| Cyp2b10 | ICLGESIAR | 594.8/745.4 |
| | (SEQ. ID NO. 6) | |
| Cyp2c29/Cyp2c37 | ICAGEGLAR | 558.8/673.4 |
| | (SEQ. ID NO. 7) | |
| Cyp2c39 | VCAGEGLAR | 551.8/673.4 |
| | (SEQ. ID NO. 8) | |
| Cyp2c40 | ICVGESLAR | 587.8/731.4 |
| | (SEQ. ID NO. 9) | |
| Cyp2d9 | SCLGEALAR | 573.8/729.4 |
| | (SEQ. ID NO. 10) | |
| Cyp2d22/Cyp2d26 | SCLGEPLAR | 586.8/642.4 |
| | (SEQ. ID NO. 11) | |
| Cyp2e1 | VCVGEGLAR | 565.8/701.4 |
| | (SEQ. ID NO. 12) | |
| Cyp2f2 | LCLGEPLAR | 599.8/642.4 |
| | (SEQ. ID NO. 13) | |
| Cyp2j5 | ACLGEQLAK | 580.3/758.4 |
| | (SEQ. ID NO. 14) | |
| Cyp3a11 | NCLGMR | 460.7/363.2 |
| | (SEQ. ID NO. 15) | |
| Cyp4a10/Cyp4a14 | NCIGK | 381.2/204.1 |
| | (SEQ. ID NO. 16) | |

The materials and method used in this example were substantially as follows.

Selection, Preparation and Quantitation of Labeled Synthetic Peptide Standards

The protein sequences of all members of the P450 protein family used in this experiment were examined. Tryptic peptide sequences containing cysteine residues were found which uniquely identified each protein isoform. Synthetic peptides of these sequences were made and labeled with CO cleavable ICAT® reagent. Peptides were synthesized using Fmoc chemistry (Applied Biosystems 433A Peptide Synthesizer, Applied Biosystems, Inc. Foster City, Calif.), derivatized using the cleavable ICAT® reagent, purified by HPLC, and their concentration quantified by amino acid analysis (Applied Biosystems 421A Derivatizer). The sixteen P450 isoforms of this experiment are listed in column 1 of Table 1. Column 2 of Table 1 list the signature peptide selected for the corresponding P450 isoform in this experiment.

Mass Analyzer System

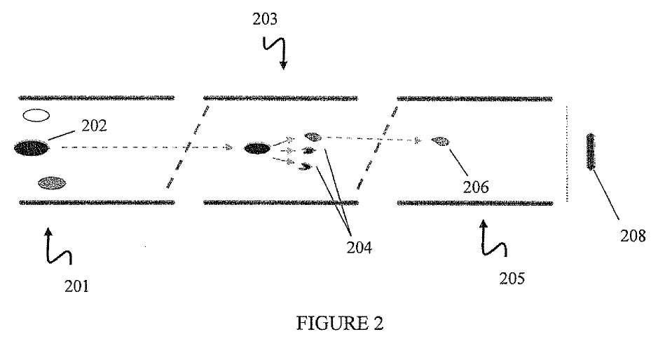

A liquid chromatography (LC) mass spectrometry (MS) system was used to analyze the standard samples and unknown samples from both control and phenobarbital induced mice. Samples were separated by reverse phase HPLC on a C18 Genesis AQ column (75 μm×10 cm, Vydac) using a 10 minute gradient (15-45% acetonitrile in 0.1% formic acid). MRM analysis was performed using a MS system with a NanoSpray™ source on a 4000 Q TRAP® system (Applied Biosystems, Inc., Foster City, Calif.) (Q1—3 Dalton (Da) mass window, Q3—1 Da mass window). A simplified schematic diagram of the mass spectrometer system used is shown in FIG. 2.

Referring to FIG. 2, a MRM scan can be conducted, for example, by setting the first mass separator 201 (in the instrument used the first mass separator is a quadrupole) to transmit the signature peptide of interest (i.e., the parent ion 202, e.g., by setting the first mass separator to transmit ions in a mass window about 3 mass units wide substantially centered on the mass of a signature peptide). In various embodiments, the collision energy can be selected to facilitate producing the selected diagnostic charged fragment of this peptide (the selected diagnostic daughter ion) in the ion fragmentor (here the ion fragmentor comprises a collision gas for conducting CID and a quadrupole 203, to facilitate, e.g., collecting ion fragments 204 and fragment ion transmittal); and the second mass separator 205 (in the instrument used the second mass separator is a quadrupole configurable as a linear ion trap) is set to transmit the diagnostic daughter ion (or ions) 206 of interest (e.g., by setting the second mass separator to transmit ions in a mass window about 1 mass unit wide substantially centered on the mass of a diagnostic daughter ion) to a detector 208 to generate an ion signal for the diagnostic daughter ion (or ions) transmitted. In these experiments the second mass separator was operated in quadrupole mode.

MRM parameters, for each signature peptide, were chosen to facilitate optimizing the signal for the selected diagnostic daughter ion (or ions) associated with that signature peptide. The dwell times (25-100 ms) used on the mass separators in this experiment and the ability to rapidly change between MRM transitions allowed multiple components in a mixture to be monitored in a single LC-MS run. Although dwell times between about 25-100 ms were used in these experiments, dwell times between about 10 ms to about 200 ms could be used depending on experimental conditions. For example, 50-100 different components can be monitored in a single LC-MS run. The parent ion m/z and daughter ion m/z MRM settings (these settings do not assume passing singly charged ions) for each signature peptide are given in column 3 of Table 1 and the approximate retention time on the column (in minutes) for each signature peptide is given in column 4 of Table 1.

Generation of Concentration Curve

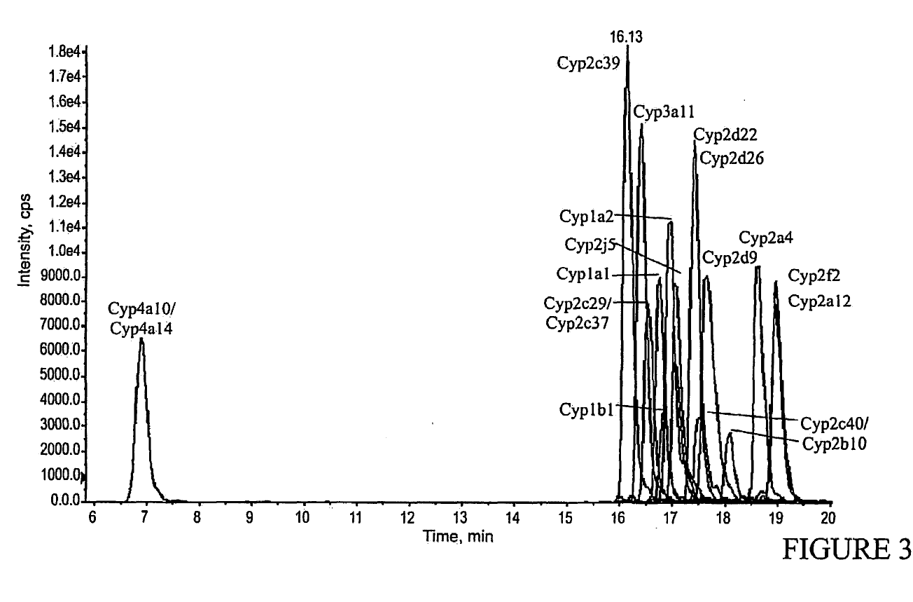

In this example, an MRM assay was developed to quantify and create concentration curves for a set of 16 synthetic peptides in a single run, using light ICAT™ reagent labeled forms of the peptides. Using a dwell time of 45 ms and monitoring 40 different transitions, the cycle time was only 2 seconds. A 10 minute gradient from 15-35% acetonitrile was used to separate the P450 peptides in time. A resultant MRM chromatogram for 3.2 fmol of each signature peptide on column is shown in FIG. 3. The y-axis in FIG. 3 corresponds to the mass spectrometry system detector signal (in counts per second (cps)) of the diagnostic daughter ion corresponding to the signature peptide of the P450 proteins noted in FIG. 3. The x-axis corresponds to the retention time (in minutes) of the signature peptide in the LC portion of the system. The chromatograms in FIG. 3 are labeled according to the P450 isoform to which they correspond. Notice that the MRM response varies for the different signature peptide sequences.

The signature peptide standard samples were used to generate the concentration curves for each peptide and act as an internal standard when measuring the unknown samples.

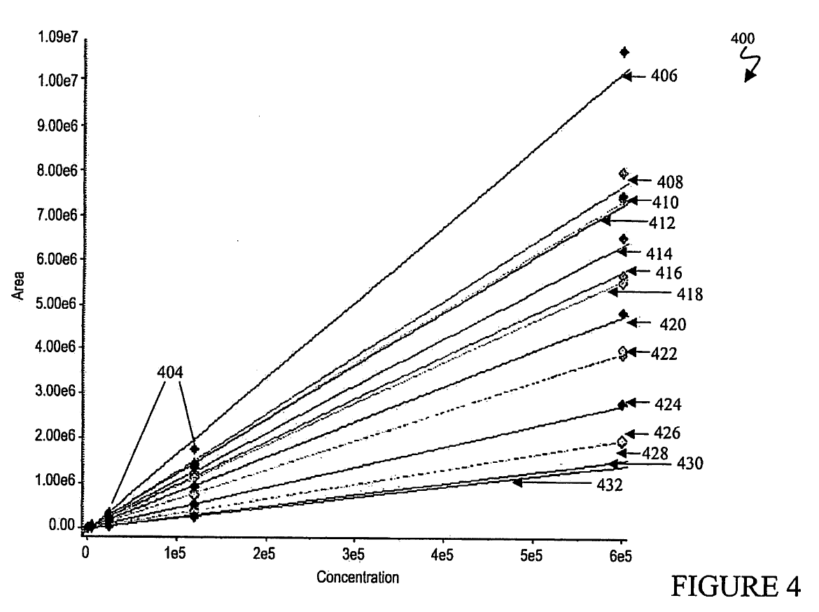

Concentration curves were measured for each synthetic light ICAT® reagent labeled peptide. The concentration curves were generated in the presence of heavy ICAT® reagent labeled microsomal proteins, to control for background and ion suppression. Examples of concentration curves generated in this experiment are shown in FIG. 4 as a plot of the diagnostic daughter ion signal area (y-axis) as a function of the signature peptide concentration (femtomoles on column) (x-axis). FIG. 4 shows concentration curves 400 for the diagnostic daughter ions of various signature peptides chosen for the various P450 isoforms in this experiment, where the filled symbols 404 represent the experimental measurements. Examples, of concentration curves for the isoforms: Cyp2d9 406, Cyp1a1 408, Cyp2b10 410, Cyp2j5 412, Cyp2d22/Cyp2d26 414, Cyp3a11 416, Cyp1b1 418, Cyp2f2 420, Cyp2a12 422, Cyp2c29/Cyp2c37 424, Cyp4a10/Cyp4a14 426, Cyp2c39 428, Cyp1a2 430, Cyp2a4 432, and Cyp2d9 432, are shown.

Labeling of Mouse Liver Microsomes

The proteins from mouse liver microsomes were extracted and the protein extracts were labeled with heavy cleavable ICAT® reagent and samples were processed according to a standard Applied Biosystems ICAT brand reagent kit protocol (e.g., Applied Biosystems Part No. 4333373Rev.A).

Quantitation of Expression

The absolute expression of a P450 isoform of this experiment, for both control (CT) and induced IND samples, can be determined, for example, by comparing the MRM peak area from the control sample with the concentration curve for the corresponding signature peptide-diagnostic daughter ion transition.

Table 2 shows the concentration ratios obtained for the sixteen P450 isoforms investigated in this experiment. In Table 2: column 1 lists the P450 isoform; column 2 lists the signature peptide selected for that isoform; column 3 gives the absolute amount of the P450 isoform expressed by the control samples in the experiment in units of femtomoles per microgram (μg) of microsomal protein; column 4 gives the ratio of induced (IND) to control (CT) expression; and column 5 qualitatively indicates whether the protein was upregulated in the IND samples relative to CT and columns 6 and 7 show respectively, the upper and lower limits of the 95% confidence intervals of the corresponding entry in column 4. In various embodiments, one or more proteins in the sample known to be unchanging (e.g., in these experiments using liver microsomes a liver protein) will be selected and signature peptide-diagnostic daughter ion transition of one or more of these proteins used provide a normalization factor between control and experimental samples.

The basal level of expression of each protein in control mouse liver microsomes was measured, and the proteins monitored showed a range of basal expression from about 1.38 to about 55.84 fmol/μg of microsomal protein. The microsomal proteins from mice, which were treated with phenobarbital, were also studied and the changes in expression of each protein in response to the drug were determined. The ratios from 4 separate experiments were averaged and the 95% confidence intervals calculated. Good reproducibility was obtained across experiments, as shown by the narrow 95% CI values. The P450 protein, Cyp2b10, showed an increase in expression upon drug treatment of about 6-fold over control. Cyp2c29/Cyp2c37 and Cyp3a11 also showed a small increase in expression, about 3-fold, whereas Cyp2d9 showed a slight decrease in expression.

TABLE 2

| | | | | | | |
|---|---|---|---|---|---|---|
| | Signature | [CT] | IND/ | | Upper | Lower |
| Protein | Peptide | fmol/μg | CT | Change | CI | CI |

| | | | | | | |
|---|---|---|---|---|---|---|
| Cyp1a1 | CIGETIGR | 5.38 | 1.03 | | 1.09 | 0.97 |
| Cyp1a2 | CIGEIPAK | 1.38 | 0.91 | | 0.95 | 0.87 |
| Cyp1b1 | CIGEELSK | 14.11 | 1.08 | | 1.23 | 0.96 |
| Cyp2a4 | YCFGEGLAR | 11.53 | 1.19 | | 1.33 | 1.06 |
| Cyp2a12 | FCLGESLAK | 15.07 | 1.0 | | 1.07 | 0.93 |
| Cyp2b10 | ICLGESIAR | 11.41 | 6.07 | up | 7.24 | 5.08 |
| Cyp2c29/ | ICAGEGLAR | 55.84 | 3.06 | up | 3.53 | 2.65 |
| Cyp2c37 | | | | | | |
| Cyp2c39 | VCAGEGLAR | 7.58 | 0.99 | | 1.05 | 0.94 |
| Cyp2c40 | ICVGESLAR | 16.15 | 0.98 | | 1.03 | 0.93 |
| Cyp2d9 | SCLGEALAR | 12.42 | 0.61 | down | 0.70 | 0.52 |
| Cyp2d22/ | SCLGEPLAR | 21.68 | 0.90 | | 0.96 | 0.86 |
| Cyp2d26 | | | | | | |
| Cyp2e1 | VCVGEGLAR | 35.13 | 0.86 | | 0.91 | 0.82 |
| Cyp2f2 | LCLGEPLAR | 21.74 | 0.75 | | 0.78 | 0.72 |
| Cyp2j5 | ACLGEQLAK | 39.05 | 0.98 | | 1.02 | 0.93 |
| Cyp3a11 | NCLGMR | 5.48 | 3.57 | up | 3.94 | 3.23 |
| Cyp4a10/ | NCIGK | 2.71 | 1.61 | | 1.97 | 1.31 |
| Cyp4a14 | | | | | | |

### Example 2

### P450 Isoforms

In this example, absolute quantitation of a set of sixteen P450 isoforms is shown where the control and induce samples were combined (without the addition of signature peptide internal standard samples) and loaded on to the chromatographic column. This example can also provide, for example, an assay for multiple P450 isoforms conductible in a single experimental run. This example used a portion of the same control and induced samples, before said samples were labeled, used in Example 1. The labeled signature peptide samples used in Example 2 were the same samples used in Example 1.

In Example 2, mouse liver microsome samples, control (CT) and phenobarbital induced (IND) were then labeled, respectively, with light cleavable and heavy cleavable ICAT reagents. Comparison of the chromatographic areas of the light and heavy peptide in a sample to the concentration curve provided quantitative information on the level of each P450 investigated in the control sample and the change in expression upon treatment with phenobarbital. Sixteen different labeled synthetic peptides, representing 16 different P450 proteins, were monitored in this experiment. The sixteen P450 proteins studied in this Example 2 are listed in column 1 of Table 1. Column 2 of Table 1 list the signature peptide selected for the corresponding P450 isoform in this experiment.

The materials and method used in this example were substantially the same as those used in Example 1 except as follows.

Mass Analyzer System

A liquid chromatography (LC) mass spectrometry (MS) system was used to analyze the standard samples and unknown samples from both control and phenobarbital induced mice. Control and Induced samples were combined, digested, and loaded onto the chromatographic column as a combined sample. Signature peptide internal standard samples were not added to this combined sample. Samples were separated by reverse phase HPLC on a C18 Genesis AQ column (75 μm×10 cm, Vydac) using a 10 minute gradient (15-45% acetonitrile in 0.1% formic acid). MRM analysis was performed as described in Example 1.

Generation of Concentration Curve

The same concentration curves described in Example 1 were used in this Example 2.

Labeling of Mouse Liver Microsomes

The proteins from mouse liver microsomes were extracted and the protein extracts were labeled with cleavable ICAT® reagent (heavy for the IND, and light for the CT) and samples were processed according to a standard Applied Biosystems ICAT brand reagent kit protocol (e.g., Applied Biosystems Part No. 4333373Rev.A).

Quantitation of Expression

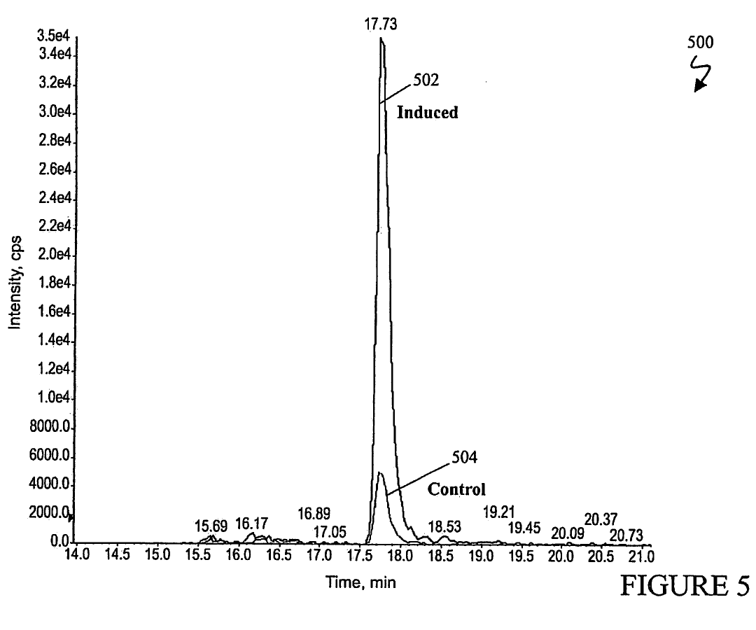

The absolute expression of a P450 isoform of this experiment, for both CT and IND samples, can be determined, for example, by comparing the MRM peak area from the control sample with the concentration curve for the corresponding signature peptide-diagnostic daughter ion transition. For example, FIG. 5 shows a MRM chromatogram 500 for the diagnostic daughter ion of the ICLGESIAR peptide (the signature peptide chosen for the Cyp2b10 isoform of P450) of Example 2, with signals from both control 502 and phenobarbital induced 504 samples. The concentration of the ICLGESIAR peptide in the CT and IND samples, and therefore the corresponding specific P450 isoform in the CT and IND samples, can be determined, for example, by comparing the MRM peak area from the control sample signal 502 with the corresponding concentration curve (e.g., FIG. 4) generated from the synthetic peptides. For example, in the control liver microsomes of this experiment, Cyp2b10 was expressed at about 2.4 fmol/μg of microsomal protein. Further, comparing the concentrations calculated from the concentration curve for the ICLGESIAR peptide from the induced sample signal 504 and the control sample signal 502, or comparing the MRM peak area for each, indicates that the expression of P450 Cyp2b10 isoform is upregulated about 7 fold upon treatment with phenobarbital.

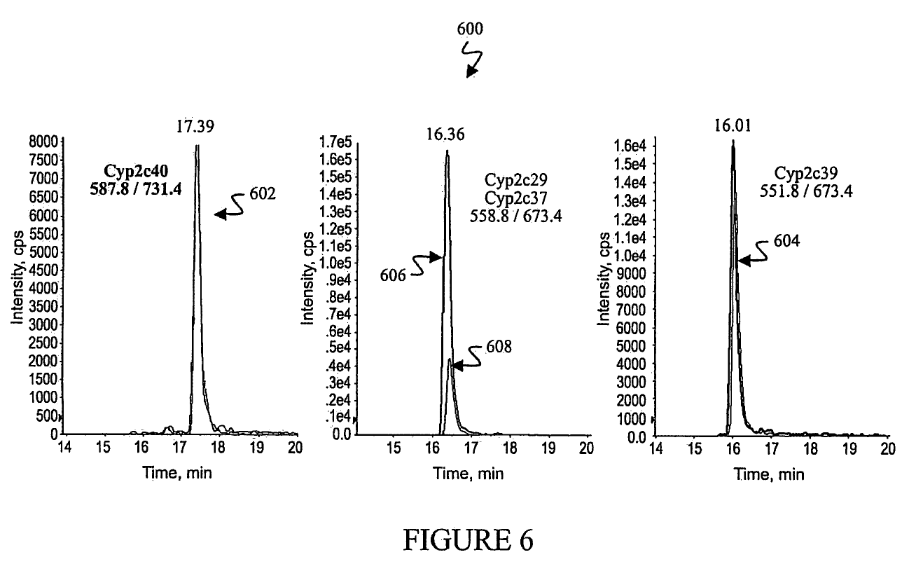

In various, embodiments, changes in expression of highly homologous proteins within the same subfamily can be determined. For example, four isoforms from the Cyp2C subfamily (Cyp2c40, Cyp2c29, Cyp2c37 and Cyp2c39) have approximately 80% sequence homology. In various embodiments, individual quantitation information can be obtained using, e.g., the specificity of the MRM method. Referring to FIG. 6, shown are MRM chromatograms 600 of control and phenobarbital induced samples, two of the isoforms (Cyp2c40 602 and Cyp2c39 604) were not substantially inducible by phenobarbitol. However, the Cyp2c29/Cyp2c37 70 isoforms showed about a 3 fold increase in expression of the induced sample 606 over the control sample 608 based on the MRM peak areas.

In various embodiments, to account for, e.g., small experimental variation in amounts of protein starting material or sample preparation, one or more proteins can be chosen to act as normalization proteins. Proteins chosen to serve as normalizations factors should remain unchanged regardless of the method of induction (e.g., drug induction) and peptide fragments of these proteins should be observed after routine sample preparation to serve as internal standards within the experiment.

Table 3 shows the normalization proteins and signature peptides used in the quantitation of P450 isozymes in Example 2. In various embodiments, normalization proteins are microsomal. In various embodiments, signature peptides of the normalization proteins are isolated tryptic fragments. In various embodiments, signature peptides are in the range between about 4 to about 30 amino acid residues in length, or between about 6 to about 15 amino acid residues in length, or between about 16 to about 30 amino acid residues in length or between about 8 to about 16 amino acid residues in length or between about 10 to about 15 amino acid residues in length.

TABLE 3

| | | | | Up- | Low- |
|---|---|---|---|---|---|
| | Signature | | | per | er |
| Protein | Peptide | MRM | Avg | CI | CI |
| Cortico- | EECALEIIK | 637.8/686.4 | 1.02 | 1.07 | 0.97 |
| steroid 11 | | | | | |
| beta-dehy- | | | | | |
| drogenase | | | | | |
| isozyme 1 | | | | | |
| Triglyeride | GCPSLAEHWK | 677.8/967.5 | 1.02 | 1.16 | 0.94 |
| transfer | | | | | |
| protein | | | | | |
| Microsomal | VFANPEDCAFGK | 791.4/1150.5 | 1.03 | 1.17 | 0.91 |
| GST | | | | | |
| Microsomal | VFANPEDCAFGK | 527.9/575.8 | 1.03 | 1.21 | 0.86 |
| GST | | | | | |

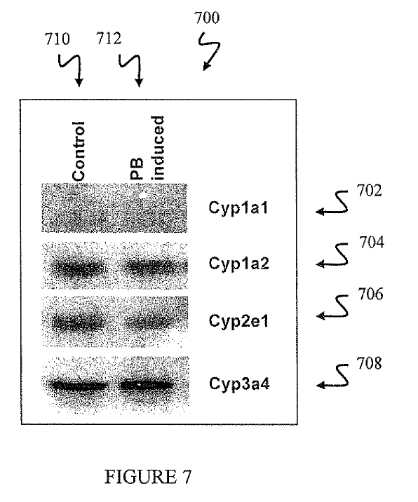

FIG. 7 illustrates the results of a Western blot analysis 700 of four of the subfamilies of P450 proteins: Cyp1a1 702, Cyp1a2 704, Cyp2e1 706 and Cyp3a4 708. Commercially available antibodies to four of the subfamilies of P450 proteins were obtained and used to analyze expressed protein levels in both the control 710 and phenobarbital induced 712 samples. Very little of the Cyp1a1 protein was observed in either sample. Cyp1a2, Cyp2e1 and Cyp3a4 proteins were observed in both samples at similar levels of expression.

### Example 3

### Plasma Proteins

In this example, forty-one of about the most abundant proteins in blood plasma were studied according to various embodiments of the present teachings and signature peptides and MRM transitions determined for the relative and/or absolute quantification of these proteins.

Mass Analyzer System

A liquid chromatography (LC) mass spectrometry (MS) system was used to analyze samples of this example. Samples were separated by reverse phase HPLC on a PepMap C18 column (75 μm×15 cm, LC Packings) using a 30 minute gradient (5-35% acetonitrile in 0.1% formic acid). MRM analysis was performed using a MS system with a NanoSpray™ source on a 4000 Q TRAP® system (Applied Biosystems, Inc., Foster City, Calif.) (Q1—0.5-0.7 m/z mass window, Q3—0.5-0.7 m/z mass window) and/or a QSTAR® system (Applied Biosystems, Inc., Foster City, Calif.) (Q1—0.5-0.7 Dalton (Da) mass window, Q3—0.5-0.7 Da mass window) as noted in this example. A simplified schematic diagram of the mass spectrometer system used is shown in FIG. 2.

### Materials and Methods

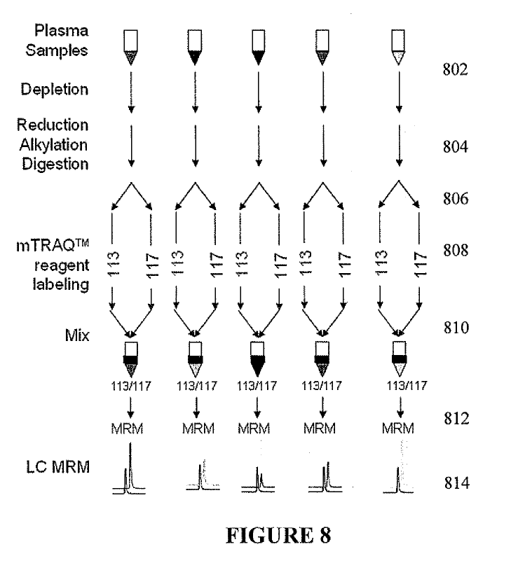

Human plasma was prepared using typical plasma handling procedures and as follows and with reference to FIG. 8. The top seven most abundant proteins were depleted from the sample using antibody depletion cartridges (Agilent MARS™ column, but other columns are available and suitable) (Step 802 FIG. 8). Remaining proteins were reduced and alkylated with iodoacetamide, then digested with trypsin (Step 804 FIG. 8); and after trypsin digestion the resulting peptide solution was desalted in preparation for labeling.

Further details on the sample preparation are as follows:

### Handling and Depletion of Human Plasma

Five 50 μL of human plasma aliquots (3500 μg of protein) were processed in parallel.

Plasma was depleted using Agilent's MARS Hu-Plasma7 (Cat.# 5188-6410) depletion column, according to the manufacturers instructions. The flow through was collected and concentrated to a volume of 100 μL. The concentration post-depletion was determined by Bradford assay.

Digestion by Trypsin (Promega) with 1:50 Enzyme to Substrate Ratio:

The depleted plasma (approximately 350%g of total protein in 25 mM NaH₂PO₄, pH 7.4, 500 mM NaCL) was denatured with Urea, reduced with fresh dithiothreitol and alkylated with iodoacetamide using standard protocols.

After ensuring a pH of 8.0, Trypsin solution was added for an enzyme to substrate ratio of 1:50 and the solution incubated according to suppliers recommendations. Following digestion, the reaction was quenched by adding formic acid to drop the pH of the solution. The total solution was then desalted using standard desalting cartridges (many types are available) according to the suppliers instructions.

A stock of human plasma was used for the assay development of this example. The stock was split into 5 equal samples and taken through a sample preparation workflow, (Steps 802 and 804). Then each was split in two (Step 806 FIG. 8) and half was labeled with a mass differential tag (light mTRAQ brand reagent) with an about 113 amu reporter ion, and the other half was labeled a mass differential tag (heavy mTRAQ brand reagent) with an about 117 amu reporter ion to create a standard sample (Step 808 FIG. 8). The labeling of the plasma samples with an mTRAQ reagent (either heavy or light) was done substantially according to Applied Biosystems typical protocol for the use of iTRAQ® brand reagents. The two sample halves were then mixed back together to create a standard sample, with heavy and light labeled peptides in about a 1:1 ratio. (Step 810 FIG. 8). This created 5 samples, which are referred to as FP1, FP2, FP3, FP4, FP5 in this example. The five samples were each then subjected to PDITM and MRM transitions were developed (signature and diagnostic daughter ions selected) based on the LC MRM triggered MS/MS data (Steps 812 FIG. 8). MRM data was processed and assessed for quality using MRM peak integration software (MultiQuant™ Software) (Steps 814 FIG. 8).

For the original MRM method development, small aliquots of each of the 5 samples, FP1, FP2, FP3, FP4, and FP5, were mixed together to create 1 combined sample (called FPcomb), to facilitate, for example, normalizing out digestion differences.

### QC of Prepared Samples—Determining Mixing Bias

To ascertain the degree, if any, of mixing bias in this example, a small portion of each of the individual samples (FP1, FP2, FP3, FP4, FP5) were run on the QSTAR® Elite system in LC/MS/MS mode to identify a population of proteins that are found in the top concentration range in plasma. About 60 proteins were confidently found repeatedly in a one-dimensional separation analysis (MS).

From these datasets of MS based quantitation using the mTRAQ peptide pairs (heavy and light), the sample mixing bias can be determined for each individual sample. For example, if the mixing of the pooled references standard (117 labeled in this example) with the individual 113 labeled sample was perfect, every peptide pair would have a 1:1 ratio. If a small excess of 117 was added over the 113, then the 113/117 ratio would be slightly below 1. This estimate can be used as a correction factor later in the MRM workflow. This strategy has the advantage of looking at all proteins in the sample, thus, for example, the median ratio of all detected pairs can be used to increase accuracy. In more traditional MRM based methods, the methods are often limited to looking for the things that have changed or are expected to change and therefore cannot ascertain if there is a sample mixing bias that needs to be corrected for. In various embodiments, the methods of the present teachings provide methods for reducing and/or correcting for mixing bias by such sample pooling.

In various embodiments, the present teachings provide a method for reducing and/or correcting for mixing bias by measuring a population of proteins that are known to be unchanging in the biological sample of interest and using those measurements to compute the sample mixing bias.

### MRM Transition Development

A combination of two strategies were employed to develop the MRMs in this example. From the large set of identified peptide spectra from the QSTAR Elite system experiment, MRM transitions for those peptides were designed from the observed charge state and the fragmentation pattern. The QSTAR system has a collision cell and therefore produces very similar fragmentation patterns to that of a triple quad or Q TRAP system which also have collision cells. These designed MRMs were then tested on the 4000 Q TRAP system using a MRM triggered MS/MS methods to detect a MRM transition, confirm the peptide identity of that MRM and to evaluate the quality of the MRM.

The quality of an MRM transition can comprise many factors including peak shape, intensity, peak width, RT, etc. In addition to testing the designed MRMs, the MRM triggered MS/MS was used in this example to find additional peptides for proteins for which a small number of peptides were found on the QSTAR system. In this example, MRM transitions were predicted in silico using tryptic cleavage rules to determine the Q1 masses of tryptic peptides and basic fragmentation rules to determine the Q3 masses of the subsequently generated MS/MS sequence ions. These MRMs transitions and triggered MS/MS were also used to test for peptide identity and MRM quality.

The present example provides a large number of MRM transitions (see Table 4 listing over 1000 such transitions) for many of the more abundant proteins in human plasma. In Table 4:

column 1 lists the protein name;

column 2 lists the SwissProt Accession number of the protein (the complete protein sequence is available from http://expasy.org/sprot/ by entering the accession number);

column 3 lists the peptide sequence targeted by the MRM (a signature peptide of the protein), sequence ID numbers for these peptides are given in Table 5;

column 4 lists whether the peptide was label with the “light” mass differential tag (light mTRAQ brand reagent) with an about 113 amu reporter ion, or with the “heavy”;

Column 5 lists the type of fragment ion generated in the collision cell and is monitored in Q3;

column 6 lists the mass the first mass analyzing quadrupole, Q1, was set to transmit, using a fixed m/z window of typically about 0.5 to about 0.7 m/z wide;

column 7 lists the mass the second mass analyzing quadrupole, Q3, was set to transmit, using a fixed m/z window of typically about 0.5 to about 0.7 m/z wide;

column 8 lists the collision energy in electron volts (eV) energy with which the ion enters the nitrogen filled collision cell, i.e., those ions transmitted by Q1;

column 9 lists the average raw peak area computed from replicate injections of the sample samples;

column 10 lists the standard deviation of the data of column 9;

column 11 lists the percent confidence value (% CV) of the data of column 9, here % CV=std dev/avg*100;

column 12 lists the normalized raw peak areas using the light/heavy MRM pair, averaged across replicate injections of the sample;

column 13 lists the standard deviation of the data of column 12;

column 14 lists the percent confidence value (% CV) of the data of column 12, here % CV=std dev/avg*100;

column 15 lists the average light/heavy MRM ratios for the four peptide fragments (diagnostic daughter ions, Q3 transmitted) for the parent peptide (signature peptide, Q1 transmitted), averaged from replicate injections of the sample;

column 16 lists the standard deviation of the data of column 15;

column 17 lists the percent confidence value (% CV) of the data of column 15, here % CV=std dev/avg*100;

Table 4 also uses the following abbreviations:

act=antichymotrypsin

approt.=apolipoprotein

bind.=binding;

prot.=protein;

gprot.=glycoprotein;

cmp=component

Comp.=Complement;

Pls.=Plasma;

Pt=precursor;

IAT=inter-alpha trypsin;

ILC=inhibitor light chain;

IP=inhibitor precursor;

IHC=inhibitor heavy chain;

rtl=retinol;

Serum para/aryl 1=Serum paraoxonase/arylesterase 1; and

Vt=Vitamin.

### Reproducibility

The reproducibility of the method of this example were also assessed. To assess reproducibility, MRM data was acquired on the four “best” MRM transitions per signature peptide determined after MRM assay development (MRM qualities assessed during method development were peak area and peak shape, MS/MS identification at MRM retention time, and other features) (with both heavy and light labels) for a total of 8 transitions for each signature peptide.

Then ten replicates were run on the mix of human plasma sample (FPcomb) and the % CV computed for the measurements. The confidence values can be computed for the raw peak areas across replicates. To conserve sample, a full loop injection was not performed in this example, reducing the injection reproducibility. In addition, this intentional use of “sloppy” protocol added extra error into the measurement to further test the ability of various embodiments of the methods of the present teachings to provide internal standard correction ability. In various embodiments, the injection method used in this example could be desirable, for example, when sample is limited, which can be the case with precious biological samples. Where desired full loop injection can be used, e.g., to provide greater accuracy.

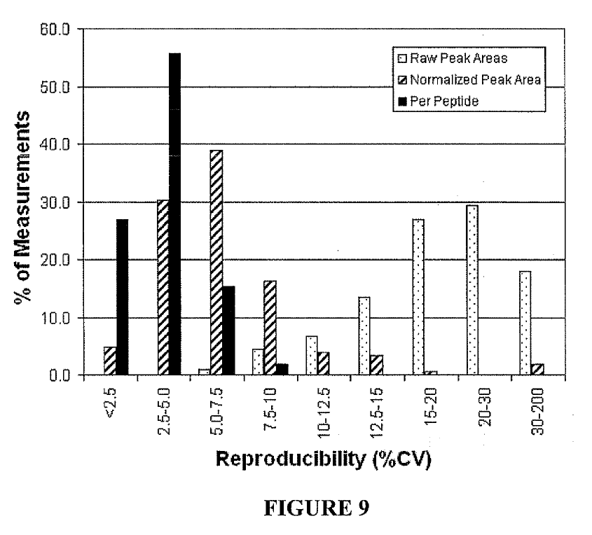

Referring to FIG. 9, the raw MRM peak areas shows a distribution of % CV centered around about 20-30% (dotted columns). Again this variation is worse than can be obtained with various embodiments of the present teachings because of injection method used. Using the heavy internal standard and computing the 113/117 ratio for each MRM (to normalize the peak area to the internal standard channel 117) the reproducibility of the measurements get much better, with a % CV centered around 5-7.5% (hashed columns). The % CV for the average ratios for each MRM pair per peptide computed across replicates are centered around 2.5-5.0% (solid columns).

The data of FIG. 9 contains data on 10 proteins, 52 peptides with 416 MRM transitions. The 10 proteins are alpha-1-antichymotryrpsin, apolipoprotein A-I, apolipoprotein A-IV, ceruloplasmin, complement factor B, complement factor H, complement C3, hemopexin, plasminogen, and fibronectin

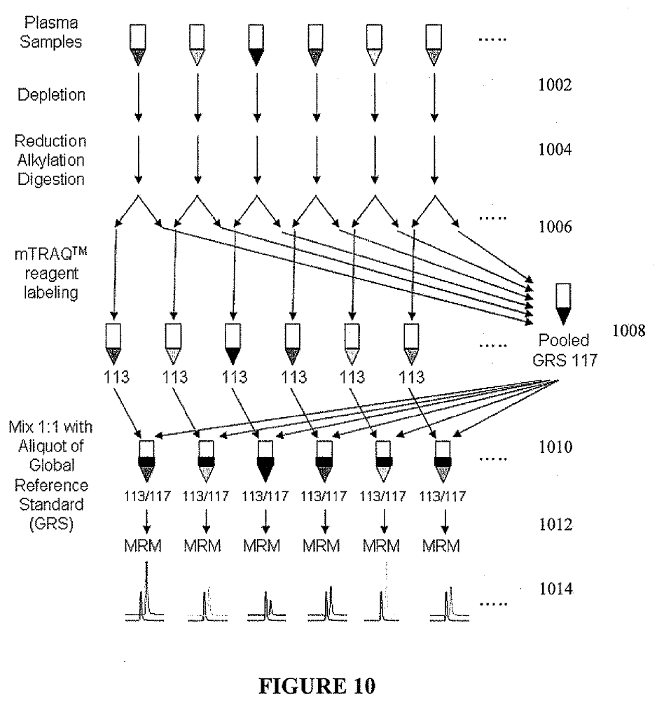

Referring to FIG. 10, it should be understood that the workflow of this example can be conducted with the use of a pooled reference sample for a standard sample. For example, as above, plasma samples are depleted, reduced, digested, desalted, etc. (Steps 1002 to 1004 in FIG. 10), then each sample is split into two substantially equal fractions (Steps 1006 in FIG. 10). A first fraction of each of the samples is combined and labeled with one of the non-isobaric chemical tages (for example the heavy tag) to form a pooled reference sample (Step 1008 in FIG. 10). The second fractions are each labeled with the other form of the label (for example, the light tag). Substantially equal portions of the pooled reference sample are then combined with each of the labeled samples to produce samples (Step 1010 in FIG. 10), which can be subjected to PDITM and MRM transitions developed (signature and diagnostic daughter ions selected) based on the MRM triggered MS/MS (Steps 1012 and 1014 in FIG. 10). MRM data was processed and assessed for quality using MRM peak integration software (MultiQuant™ Software) (Steps 814 FIG. 8).

### Example 5

### Lung Metastasis

This example uses various embodiments of the present teachings to develop and run methods for assessing changes in a biological system based on a comparison of the relative change in concentrations of two or more proteins in one or more of the two or more samples to the concentration of two or more corresponding proteins in one or more of the standard samples.

Despite advances in diagnosis and treatment, cancer mortality rates have not declined appreciably over the last decade and some cancers, such as lung cancer, are characterized by an increase in mortality. Mortality is mainly attributed to cancer metastases, for which no effective treatment is currently available. There is a critical need for the early detection of biomarkers of cancer, especially biomarkers that would enable the differentiation between localized cancers and more aggressive forms of the disease that are prone to metastases. The present example provides methods for the relative quantification of proteins involved in metastasis, specifically those related to two pathways that are important in metastasis (the ErbB2 cell proliferation and the integrin activation pathways). In various embodiments, the methods of the present example allow for substantially simultaneous analysis of these two pathways by studying two different lung cancer cell lines, grown under two different conditions. In the present example, the expression of these proteins will be monitored in multiple cell lines to verify these proteins as metastasis biomarker candidates.

### Materials and Methods

### Preparation of Cells

The control cells were Lewis lung cancer cells (LLC-AP2). A variant of these cells was created by tranfection in order to cause the cells to overexpress ErbB2. The metastatic potential of these cells was evaluated by implanting the cells into the mammary fat pad of SCID mice. Lung tumors resulting from metastasis were harvested and subcultured to provide a low metastatic variant (LLC-ErbB2-P2) and a highly metastatic variant (LLC-ErbB2-M4). The cell lines were cultured in the presence and absence of fibronectin. Cells were lysed, proteins were isolated (100 μg), digested with trypsin, labeled with mTRAQ™ brand reagents (Applied Biosystems, Foster City, Calif.) according to the standard Applied Biosystems protocol.

### Chromatography

The labeled samples were separated into 40 fractions by strong cation exchange (100×2.1 mm, 5 μm, 200A, Polysulfoethyl A column, 200 μl/min, 10-500 mM ammonium formate pH 3). The SCX fractions were further analysed by LC-MS/MS using a C18 column (75 μm×15 cm, LC Packings; 5-30% acetonitrile over 30 min) on a Tempo™ LC System and analyzed by MS.

### Mass Spectrometry

MRM triggered MS/MS was performed on the 4000 Q TRAP® system.

### Data Processing

Identification of MRM triggered MS/MS data was performed using the Paragon™ Database Search Algorithm and Pro Group™ Algorithm in ProteinPilot™ Software (Applied Biosystems, Foster City, Calif.). The MRM peaks were integrated with MultiQuant™ Software (Applied Biosystems, Foster City, Calif.).

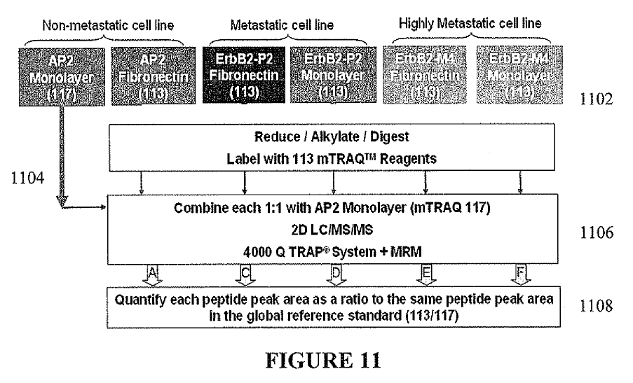

Referring to FIG. 11, five different cell lines/growth conditions were analyzed in a multiplex manner and a sixth cell line used as a reference sample. The five cell lines/growth conditions analyzed, LLC-AP2 cultured in the presence of fibronectin (AP2 fibronectin); LLC-ErbB2-P2 cultured in the presence of fibronectin (ErbB2-P2 fibronectin); LLC-ErbB2-P2 cultured in the absence of fibronectin (ErbB2-P2 monolayer) LLC-ErbB2-M4 cultured in the presence of fibronectin (ErbB2-M4 fibronectin); and LLC-ErbB2-M4 cultured in the absence of fibronectin (ErbB2-M4 monolayer) were each labeled with label 113 from a set of mTRAQ™ brand reagents; after the cells, were lysed, the proteins isolated and digested (Step 1102). The reference sample LLC-AP2 cultured in the absence of fibronectin (AP2 monolayer) was labeled with label 117 from the set of mTRAQ™ brand reagents, after the cells, were lysed, the proteins isolated and digested (Step 1104).

Each of the 113 labeled samples were then combined with a substantially equal amount of the reference sample in a 1:1 ratio (Step 1106) to produce five combined samples for analysis. Each of these five samples was then analyzed by LC MRM triggered MS/MS using a 4000 Q TRAP system according to various embodiments of the present teachings to obtain quantitative information on the protein expression relative to the reference sample (Step 1108).

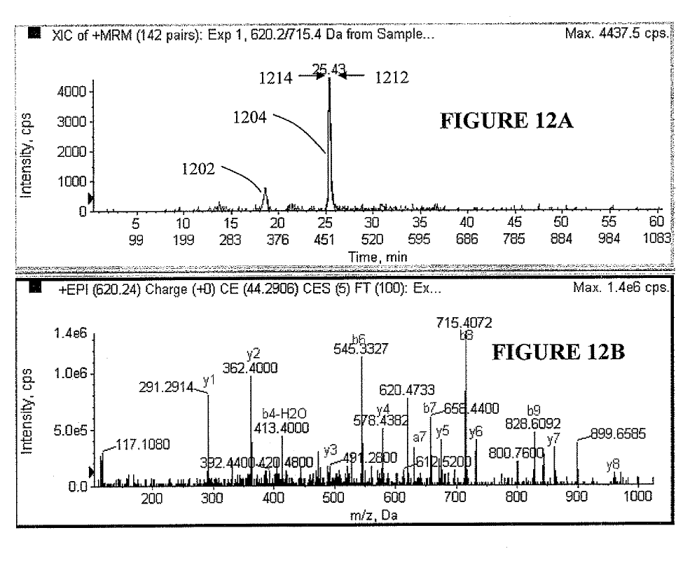

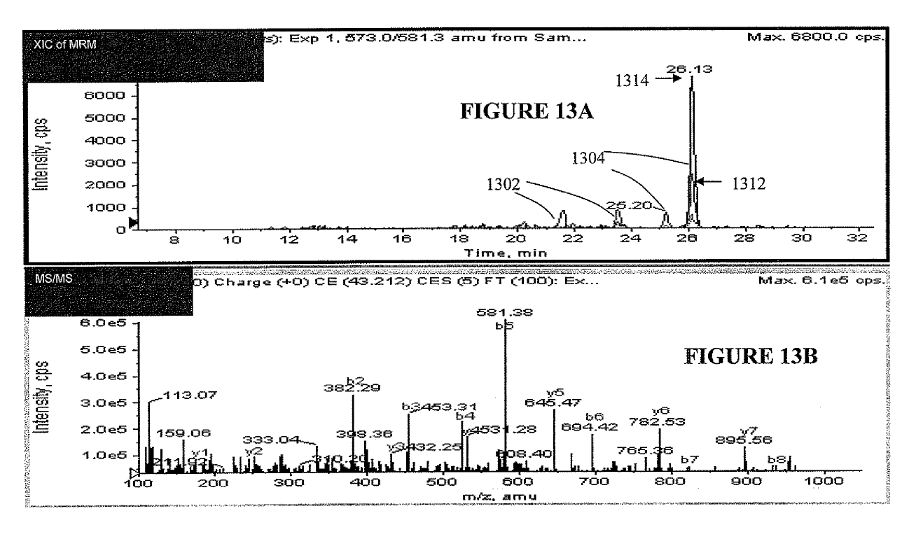

FIGS. 12A and 13A present ion current as a function of time for a fixed MRM transition. In FIG. 12A the blue trace (1202) is for the MRM transition Q1/Q3=620.2/715.4 and the red trace (1204) is for the MRM transition Q1/Q3=620.2/545.3 both corresponding to a signature peptide of GAGTGGLGLAVEGPSEAK (SEQ. ID. NO. 176) for filamin A. The arrows 1212 and 1214 designate the approximate maximum signal for the Q1/Q3=620.2/715.4 trace (blue trace) and Q1/Q3=620.2/545.3 (red trace) respectively. In FIG. 13A the blue trace (1302) if for the MRM transition Q1/Q3=573.0/581.3 and the red trace (1304) is for the MRM transition Q1/Q3=573.0/645.5, both corresponding to a signature peptide of LQAAGIQLHNVWAR (SEQ. ID. NO. 177) for laminin alpha 5. The arrows 1312 and 1314 designate the approximate maximum signal for the Q1/Q3=573.0/581.3 trace (blue trace) and Q1/Q3=573.0/645.5 (red trace) respectively.

FIGS. 12B and 13B present fragmentation spectra of the signature peptide of 12A and 13A, respectively, that is the ion transmitted by Q1. In FIG. 12B the collision energy was about 44 eV and in FIG. 13B about 43 eV. The fragmentation spectra can be used, for example, to determine and/or confirm the structure of the signature peptide and/or further refine the MRM transitions for a final assay.

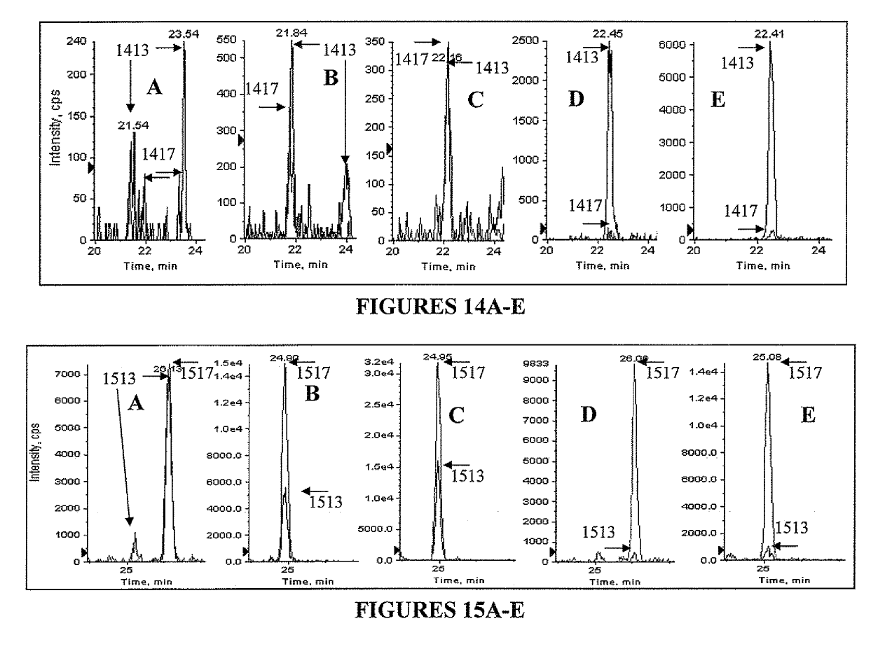

FIGS. 14A-E and 15A-E present ion current data for a fixed MRM transition for a signature peptide, respectively, of filamin A (FIGS. 14A-E) and laminin alpha 5 (FIGS. 15A-E). In FIGS. 14A-E, the signature peptide was GAGTGGLGLAVEGPSEAK (SEQ. ID. NO. 176) and the MRM transition used was Q1/Q3=617.6/654.4 for the light peptide. In FIGS. 15A-E, the signature peptide was LQAAGIQLHNVWAR (SEQ. ID. NO. 177) and the MRM transition used was Q1/Q3=573.0/581.3 for the light peptide.

The set of arrows 1413 in FIGS. 14A-E indicate the approximate peak of the analyte traces, 113 labeled sample, (blue traces) and the set of arrows 1417 indicate the approximate peak of the reference sample traces, 117 labeled, (red traces). The set of arrows 1513 in FIGS. 15A-E indicate the approximate peak of the analyte traces, 113 labeled sample, (blue traces) and the set of arrows 1517 indicate the approximate peak of the reference sample traces, 117 labeled, (red traces).

The reference sample in FIGS. 14A-15E was AP2 monolayer. The analyte samples, 113 labeled, are: AP2 fibronectin in FIGS. 14A and 15A; ErbB2-P2 fibronectin in FIGS. 14B and 15B; ErbB2-P2 monolayer in FIGS. 14C and 15C; ErbB2-M4 fibronectin in FIGS. 14D and 15D; and ErbB2-M4 monolayer in FIGS. 14E and 15E.

FIGS. 14A-E demonstrate the increase in expression of filimin A in highly metastatic cells (FIGS. 14D and E) observing a large increase (×10) relative to the reference sample (red trace). Minimal change was observed in non-metastatic or low metastatic cells (FIGS. 14A-C).

FIGS. 15A-E demonstrate the decrease in expression of laminin alpha 5 in highly metastatic cells (FIGS. 15D and E) observing a large decrease (×10) relative to the reference sample (red trace). A two-fold decrease in expression was also observed in in low metastatic cells (FIGS. 15B and 15C).

TABLE 4

| Protein Level | | Peptide Level | | | | | | | | | | | | | | |
|---|---|---|---|---|---|---|---|---|---|---|---|---|---|---|---|---|
| | Total | | Total | | | | | | | | MRM | | | | | |
| | Proteins | | Peptides | | | | | MRM | | | Ratios | | | Peptide | | |
| | 41 | | 147 | | MRM | | | areas | | | (113/117) | | | ratios | | |
| Protein Name | Access.# | Peptide Sequence | Label | Ion | Q1 | Q3 | CE | avg ratio | Std | % CV | avg ratio | Std | % CV | avg ratio | Std | % CV |
| α-1-acid gprot. 1 | P02763 | SDVVYTDWK | 113 | b3 | 464.9 | 442.2 | 41 | 1336965 | 175988 | 13.2 | 0.84 | 0.02 | 2.3 | 0.93 | 0.12 | 12.8 |
| α-1-acid gprot. 1 | P02763 | SDVVYTDWK | 113 | y2 | 464.9 | 473.3 | 41 | 596964 | 82207 | 13.8 | 0.89 | 0.04 | 4.1 | | | |
| α-1-acid gprot. 1 | P02763 | SDVVYTDWK | 113 | y2 | 696.9 | 473.3 | 53 | 3629660 | 446622 | 12.3 | 1.10 | 0.05 | 4.6 | | | |
| α-1-acid gprot. 1 | P02763 | SDVVYTDWK | 113 | b4 | 464.9 | 541.3 | 41 | 139392 | 20590 | 14.8 | 0.87 | 0.04 | 4.5 | | | |
| α-1-acid gprot. 1 | P02763 | SDVVYTDWK | 117 | b3 | 467.6 | 446.2 | 41 | 1585957 | 223990 | 14.1 | | | | | | |
| α-1-acid gprot. 1 | P02763 | SDVVYTDWK | 117 | y2 | 467.6 | 477.3 | 41 | 675417 | 100196 | 14.8 | | | | | | |
| α-1-acid gprot. 1 | P02763 | SDVVYTDWK | 117 | y2 | 700.9 | 477.3 | 53 | 3307652 | 495522 | 15.0 | | | | | | |
| α-1-acid gprot. 1 | P02763 | SDVVYTDWK | 117 | b4 | 467.6 | 545.3 | 41 | 158852 | 17904 | 11.3 | | | | | | |
| α-1-acid gprot. 1 | P02763 | YVGGQEHFAHLLILR | 113 | b5 | 474.0 | 645.3 | 42 | 546054 | 54814 | 10.0 | 0.87 | 0.03 | 3.8 | 1.06 | 0.15 | 14.3 |
| α-1-acid gprot. 1 | P02763 | YVGGQEHFAHLLILR | 113 | b5 | 631.7 | 645.3 | 50 | 437220 | 96915 | 22.2 | 1.17 | 0.05 | 4.5 | | | |
| α-1-acid gprot. 1 | P02763 | YVGGQEHFAHLLILR | 113 | y6 | 631.7 | 764.5 | 50 | 138581 | 28217 | 20.4 | 1.20 | 0.05 | 3.8 | | | |
| α-1-acid gprot. 1 | P02763 | YVGGQEHFAHLLILR | 113 | b6 | 474.0 | 774.4 | 42 | 379141 | 29400 | 7.8 | 1.02 | 0.04 | 4.3 | | | |
| α-1-acid gprot. 1 | P02763 | YVGGQEHFAHLLILR | 117 | b5 | 475.0 | 649.3 | 42 | 632106 | 82196 | 13.0 | | | | | | |
| α-1-acid gprot. 1 | P02763 | YVGGQEHFAHLLILR | 117 | b5 | 633.0 | 649.3 | 50 | 374603 | 79155 | 21.1 | | | | | | |
| α-1-acid gprot. 1 | P02763 | YVGGQEHFAHLLILR | 117 | y6 | 633.0 | 764.5 | 50 | 116406 | 26126 | 22.4 | | | | | | |
| α-1-acid gprot. 1 | P02763 | YVGGQEHFAHLLILR | 117 | b6 | 475.0 | 778.4 | 42 | 373691 | 43713 | 11.7 | | | | | | |
| α-1B-gprot. | P04217 | ATWSGAVLAGR | 113 | y6 | 614.8 | 730.4 | 49 | 356323 | 40696 | 11.4 | 0.84 | 0.02 | 2.7 | 1.09 | 0.17 | 15.8 |
| α-1B-gprot. | P04217 | ATWSGAVLAGR | 113 | y4 | 614.8 | 416.3 | 49 | 259176 | 34956 | 13.5 | 1.19 | 0.03 | 2.9 | | | |
| α-1B-gprot. | P04217 | ATWSGAVLAGR | 113 | b6 | 614.8 | 714.4 | 49 | 209983 | 24124 | 11.5 | 1.12 | 0.04 | 3.5 | | | |
| α-1B-gprot. | P04217 | ATWSGAVLAGR | 113 | y6 | 614.8 | 916.5 | 49 | 108340 | 14150 | 13.1 | 1.21 | 0.09 | 7.3 | | | |
| α-1B-gprot. | P04217 | ATWSGAVLAGR | 117 | y6 | 616.8 | 730.4 | 49 | 424597 | 49630 | 11.7 | | | | | | |
| α-1B-gprot. | P04217 | ATWSGAVLAGR | 117 | y4 | 616.8 | 416.3 | 49 | 217586 | 28283 | 13.0 | | | | | | |
| α-1B-gprot. | P04217 | ATWSGAVLAGR | 117 | b6 | 616.8 | 718.4 | 49 | 186907 | 20680 | 11.1 | | | | | | |
| α-1B-gprot. | P04217 | ATWSGAVLAGR | 117 | y6 | 616.8 | 916.5 | 49 | 89151 | 9093 | 10.2 | | | | | | |
| α-1B-gprot. | P04217 | LLELTGPK | 113 | y3 | 575.9 | 441.3 | 47 | 452285 | 85496 | 18.9 | 1.15 | 0.10 | 8.6 | 1.30 | 0.14 | 10.9 |
| α-1B-gprot. | P04217 | LLELTGPK | 113 | y4 | 575.9 | 542.3 | 47 | 191482 | 31502 | 16.5 | 1.24 | 0.08 | 6.2 | | | |
| α-1B-gprot. | P04217 | LLELTGPK | 113 | b3 | 575.9 | 496.3 | 47 | 316272 | 37255 | 11.8 | 1.48 | 0.08 | 5.5 | | | |
| α-1B-gprot. | P04217 | LLELTGPK | 113 | y5 | 575.9 | 655.4 | 47 | 59504 | 11540 | 19.4 | 1.31 | 0.13 | 10.0 | | | |
| α-1B-gprot. | P04217 | LLELTGPK | 117 | y3 | 579.9 | 445.3 | 47 | 395730 | 75937 | 19.2 | | | | | | |
| α-1B-gprot. | P04217 | LLELTGPK | 117 | y4 | 579.9 | 546.3 | 47 | 154978 | 24520 | 15.8 | | | | | | |
| α-1B-gprot. | P04217 | LLELTGPK | 117 | b3 | 579.9 | 500.3 | 47 | 213905 | 28595 | 13.4 | | | | | | |
| α-1B-gprot. | P04217 | LLELTGPK | 117 | y5 | 579.9 | 659.4 | 47 | 45442 | 8623 | 19.0 | | | | | | |
| α-1B-gprot. | P04217 | SGLSTGWTQLSK | 113 | b3 | 515.6 | 398.2 | 44 | 648709 | 40392 | 6.2 | 1.21 | 0.04 | 2.9 | 1.22 | 0.03 | 2.3 |
| α-1B-gprot. | P04217 | SGLSTGWTQLSK | 113 | y3 | 515.6 | 487.3 | 44 | 240160 | 20081 | 8.4 | 1.22 | 0.07 | 6.1 | | | |
| α-1B-gprot. | P04217 | SGLSTGWTQLSK | 113 | b4 | 515.6 | 485.3 | 44 | 171800 | 10100 | 5.9 | 1.19 | 0.08 | 6.6 | | | |
| α-1B-gprot. | P04217 | SGLSTGWTQLSK | 113 | b6 | 515.6 | 643.3 | 44 | 76586 | 9135 | 11.9 | 1.26 0.11 | 8.4 | | | | |
| α-1B-gprot. | P04217 | SGLSTGWTQLSK | 117 | b3 | 518.3 | 402.2 | 44 | 535066 | 35401 | 6.6 | | | | | | |
| α-1B-gprot. | P04217 | SGLSTGWTQLSK | 117 | y3 | 518.3 | 491.3 | 44 | 197967 | 20576 | 10.4 | | | | | | |
| α-1B-gprot. | P04217 | SGLSTGWTQLSK | 117 | b4 | 518.3 | 489.3 | 44 | 145281 | 11809 | 8.1 | | | | | | |
| α-1B-gprot. | P04217 | SGLSTGWTQLSK | 117 | b6 | 518.3 | 647.3 | 44 | 61193 | 7112 | 11.6 | | | | | | |
| α-2-macroglobulin | P01023 | AIGYLNTGYQR | 113 | b4 | 698.4 | 545.3 | 53 | 715449 | 112375 | 15.7 | 0.96 | 0.05 | 4.9 | 1.01 | 0.04 | 3.6 |
| α-2-macroglobulin | P01023 | AIGYLNTGYQR | 113 | y6 | 698.4 | 738.3 | 53 | 333473 | 52956 | 15.9 | 1.05 | 0.03 | 3.1 | | | |
| α-2-macroglobulin | P01023 | AIGYLNTGYQR | 113 | y9 | 698.4 | 1071.5 | 53 | 411453 | 53412 | 13.0 | 1.03 | 0.04 | 4.1 | | | |
| α-2-macroglobulin | P01023 | AIGYLNTGYQR | 113 | y7 | 698.4 | 851.4 | 53 | 334344 | 49442 | 14.8 | 1.02 | 0.05 | 4.7 | | | |
| α-2-macroglobulin | P01023 | AIGYLNTGYQR | 117 | b4 | 700.4 | 549.3 | 53 | 745909 | 127552 | 17.1 | | | | | | |
| α-2-macroglobulin | P01023 | AIGYLNTGYQR | 117 | y6 | 700.4 | 738.3 | 53 | 318337 | 51898 | 16.3 | | | | | | |
| α-2-macroglobulin | P01023 | AIGYLNTGYQR | 117 | y9 | 700.4 | 1071.5 | 53 | 402179 | 62651 | 15.6 | | | | | | |
| α-2-macroglobulin | P01023 | AIGYLNTGYQR | 117 | y7 | 700.4 | 851.4 | 53 | 330889 | 56534 | 17.1 | | | | | | |
| α-2-macroglobulin | P01023 | NEDSLVFVQTDK | 113 | b4 | 558.9 | 586.2 | 46 | 1365301 | 70184 | 5.1 | 0.80 | 0.02 | 2.2 | 0.93 | 0.09 | 10.1 |
| α-2-macroglobulin | P01023 | NEDSLVFVQTDK | 113 | b4 | 837.9 | 586.2 | 60 | 283693 | 47726 | 16.8 | 1.03 | 0.08 | 8.2 | | | |
| α-2-macroglobulin | P01023 | NEDSLVFVQTDK | 113 | y4 | 558.9 | 631.3 | 46 | 80266 | 10031 | 12.5 | 0.95 | 0.09 | 9.4 | | | |
| α-2-macroglobulin | P01023 | NEDSLVFVQTDK | 113 | b5 | 558.9 | 699.3 | 46 | 548701 | 45839 | 8.4 | 0.92 | 0.02 | 2.0 | | | |
| α-2-macroglobulin | P01023 | NEDSLVFVQTDK | 117 | b4 | 561.6 | 590.3 | 46 | 1697743 | 92634 | 5.5 | | | | | | |
| α-2-macroglobulin | P01023 | NEDSLVFVQTDK | 117 | b4 | 841.9 | 590.3 | 60 | 275185 | 43653 | 15.9 | | | | | | |
| α-2-macroglobulin | P01023 | NEDSLVFVQTDK | 117 | y4 | 561.6 | 635.3 | 46 | 84950 | 11830 | 13.9 | | | | | | |
| α-2-macroglobulin | P01023 | NEDSLVFVQTDK | 117 | b5 | 561.6 | 703.3 | 46 | 594746 | 52699 | 8.9 | | | | | | |
| α-2-macroglobulin | P01023 | SSGSLLNNAIK | 113 | b5 | 461.9 | 572.3 | 41 | 113312 | 21215 | 18.7 | 0.96 | 0.34 | 36.0 | 1.06 | 0.07 | 6.5 |
| α-2-macroglobulin | P01023 | SSGSLLNNAIK | 113 | b5 | 692.4 | 572.3 | 53 | 607841 | 245497 | 40.4 | 1.11 | 0.41 | 36.8 | | | |
| α-2-macroglobulin | P01023 | SSGSLLNNAIK | 113 | b6 | 692.4 | 685.4 | 53 | 289710 | 119247 | 41.2 | 1.08 | 0.40 | 37.3 | | | |
| α-2-macroglobulin | P01023 | SSGSLLNNAIK | 113 | y5 | 692.4 | 699.4 | 53 | 197231 | 88937 | 45.1 | 1.09 | 0.39 | 35.5 | | | |
| α-2-macroglobulin | P01023 | SSGSLLNNAIK | 117 | b5 | 464.6 | 576.3 | 41 | 104843 | 23087 | 22.0 | | | | | | |
| α-2-macroglobulin | P01023 | SSGSLLNNAIK | 117 | b5 | 696.4 | 576.3 | 53 | 493483 | 207482 | 42.0 | | | | | | |
| α-2-macroglobulin | P01023 | SSGSLLNNAIK | 117 | b6 | 696.4 | 689.4 | 53 | 245674 | 110627 | 45.0 | | | | | | |
| α-2-macroglobulin | P01023 | SSGSLLNNAIK | 117 | y5 | 696.4 | 703.4 | 53 | 161791 | 80651 | 49.8 | | | | | | |
| α-1-act | P01011 | ADLSGITGAR | 113 | b5 | 550.8 | 584.3 | 46 | 403246 | 40474 | 10.0 | 1.06 | 0.05 | 4.3 | 0.98 | 0.05 | 5.5 |
| α-1-act | P01011 | ADLSGITGAR | 113 | y8 | 550.8 | 774.4 | 46 | 386825 | 67910 | 17.6 | 0.95 | 0.02 | 2.4 | | | |
| α-1-act | P01011 | ADLSGITGAR | 113 | y7 | 550.8 | 661.4 | 46 | 348033 | 56946 | 16.4 | 0.95 | 0.03 | 3.6 | | | |
| α-1-act | P01011 | ADLSGITGAR | 113 | y6 | 550.8 | 574.3 | 46 | 200627 | 25569 | 12.7 | 0.98 | 0.03 | 3.0 | | | |
| α-1-act | P01011 | ADLSGITGAR | 117 | b5 | 552.8 | 588.3 | 46 | 379679 | 42554 | 11.2 | | | | | | |
| α-1-act | P01011 | ADLSGITGAR | 117 | y8 | 552.8 | 774.4 | 46 | 409103 | 72331 | 17.7 | | | | | | |
| α-1-act | P01011 | ADLSGITGAR | 117 | y7 | 552.8 | 661.4 | 46 | 365742 | 59638 | 16.3 | | | | | | |
| α-1-act | P01011 | ADLSGITGAR | 117 | y6 | 552.8 | 574.3 | 46 | 205354 | 26778 | 13.0 | | | | | | |
| α-1-act | P01011 | AVLDVFEEGTEASAATAVK | 113 | b3 | 730.0 | 424.3 | 55 | 270793 | 94788 | 35.0 | 1.07 | 0.07 | 6.8 | 1.10 | 0.05 | 4.9 |
| α-1-act | P01011 | AVLDVFEEGTEASAATAVK | 113 | b4 | 730.0 | 539.3 | 55 | 1306821 | 465571 | 35.6 | 1.17 | 0.06 | 4.9 | | | |
| α-1-act | P01011 | AVLDVFEEGTEASAATAVK | 113 | b4 | 1094.6 | 539.3 | 73 | 602287 | 165304 | 27.4 | 1.12 | 0.07 | 6.6 | | | |
| α-1-act | P01011 | AVLDVFEEGTEASAATAVK | 113 | b5 | 1094.6 | 638.4 | 73 | 118253 | 37247 | 31.5 | 1.04 | 0.10 | 9.3 | | | |
| α-1-act | P01011 | AVLDVFEEGTEASAATAVK | 117 | b3 | 732.7 | 428.3 | 55 | 254542 | 93846 | 36.9 | | | | | | |
| α-1-act | P01011 | AVLDVFEEGTEASAATAVK | 117 | b4 | 732.7 | 543.3 | 55 | 1126891 | 409417 | 36.3 | | | | | | |
| α-1-act | P01011 | AVLDVFEEGTEASAATAVK | 117 | b4 | 1098.6 | 543.3 | 73 | 536417 | 126191 | 23.5 | | | | | | |
| α-1-act | P01011 | AVLDVFEEGTEASAATAVK | 117 | b5 | 1098.6 | 642.4 | 73 | 113592 | 34094 | 30.0 | | | | | | |
| α-1-act | P01011 | EIGELYLPK | 113 | y4 | 671.4 | 569.3 | 52 | 1303961 | 213525 | 16.4 | 0.99 | 0.03 | 3.5 | 0.99 | 0.03 | 3.4 |
| α-1-act | P01011 | EIGELYLPK | 113 | y3 | 671.4 | 440.3 | 52 | 642044 | 107596 | 16.8 | 0.97 | 0.04 | 4.5 | | | |
| α-1-act | P01011 | EIGELYLPK | 113 | y5 | 671.4 | 682.4 | 52 | 569092 | 98898 | 17.4 | 0.96 | 0.05 | 4.7 | | | |
| α-1-act | P01011 | EIGELYLPK | 113 | y3 | 671.4 | 497.3 | 52 | 256032 | 41227 | 16.1 | 1.03 | 0.04 | 4.2 | | | |
| α-1-act | P01011 | EIGELYLPK | 117 | y4 | 675.4 | 573.3 | 52 | 1317737 | 204919 | 15.6 | | | | | | |
| α-1-act | P01011 | EIGELYLPK | 117 | y3 | 675.4 | 444.3 | 52 | 662975 | 104313 | 15.7 | | | | | | |
| α-1-act | P01011 | EIGELYLPK | 117 | y5 | 675.4 | 686.4 | 52 | 596607 | 116768 | 19.6 | | | | | | |
| α-1-act | P01011 | EIGELYLPK | 117 | y3 | 675.4 | 501.4 | 52 | 248229 | 44326 | 17.9 | | | | | | |
| α-1-act | P01011 | ITLLSALVETR | 113 | y7 | 678.4 | 775.4 | 52 | 454690 | 84095 | 18.5 | 1.29 | 0.13 | 10.1 | 1.18 | 0.20 | 16.7 |
| α-1-act | P01011 | ITLLSALVETR | 113 | y7 | 678.4 | 888.5 | 52 | 223591 | 50565 | 22.6 | 1.25 | 0.10 | 7.9 | | | |
| α-1-act | P01011 | ITLLSALVETR | 113 | y10 | 678.4 | 1102.6 | 52 | 270825 | 59675 | 22.0 | 1.29 | 0.06 | 4.4 | | | |
| α-1-act | P01011 | ITLLSALVETR | 113 | y4 | 678.4 | 504.3 | 52 | 132545 | 30046 | 22.7 | 0.88 | 0.06 | 7.3 | | | |
| α-1-act | P01011 | ITLLSALVETR | 117 | y7 | 680.4 | 775.4 | 52 | 357778 | 84519 | 23.6 | | | | | | |
| α-1-act | P01011 | ITLLSALVETR | 117 | y7 | 680.4 | 888.5 | 52 | 179311 | 40552 | 22.6 | | | | | | |
| α-1-act | P01011 | ITLLSALVETR | 117 | y10 | 680.4 | 1102.6 | 52 | 210318 | 44649 | 21.2 | | | | | | |
| α-1-act | P01011 | ITLLSALVETR | 117 | y4 | 680.4 | 504.3 | 52 | 149935 | 29896 | 19.9 | | | | | | |
| α-1-act | P01011 | LYGSEAFATDFQDSAAAK | 113 | y3 | 724.7 | 429.3 | 54 | 477151 | 88154 | 18.5 | 1.09 | 0.06 | 5.6 | 1.10 | 0.07 | 6.4 |
| α-1-act | P01011 | LYGSEAFATDFQDSAAAK | 113 | b3 | 724.7 | 474.3 | 54 | 202849 | 32885 | 16.2 | 1.11 | 0.07 | 6.7 | | | |
| α-1-act | P01011 | LYGSEAFATDFQDSAAAK | 113 | y5 | 724.7 | 690.3 | 54 | 624212 | 100282 | 16.1 | 1.02 | 0.05 | 5.4 | | | |
| α-1-act | P01011 | LYGSEAFATDFQDSAAAK | 113 | b5 | 1086.5 | 690.3 | 72 | 139653 | 30750 | 22.0 | 1.19 | 0.10 | 8.7 | | | |
| α-1-act | P01011 | LYGSEAFATDFQDSAAAK | 117 | y3 | 727.4 | 433.3 | 54 | 436170 | 78601 | 18.0 | | | | | | |
| α-1-act | P01011 | LYGSEAFATDFQDSAAAK | 117 | b3 | 727.4 | 478.3 | 54 | 183851 | 29965 | 16.3 | | | | | | |
| α-1-act | P01011 | LYGSEAFATDFQDSAAAK | 117 | y5 | 727.4 | 694.4 | 54 | 611706 | 102201 | 16.7 | | | | | | |
| α-1-act | P01011 | LYGSEAFATDFQDSAAAK | 117 | b5 | 1090.5 | 694.4 | 73 | 116024 | 18393 | 15.9 | | | | | | |
| AMBP prot. IAT ILC | P02775 | AFIQLWAFDAVK | 113 | b3 | 563.6 | 472.3 | 46 | 382494 | 68405 | 17.9 | 0.97 | 0.04 | 4.3 | 1.01 | 0.14 | 14.0 |
| AMBP prot. IAT ILC | P02775 | AFIQLWAFDAVK | 113 | b4 | 563.6 | 600.4 | 46 | 216219 | 34001 | 15.7 | 0.87 | 0.02 | 2.8 | | | |
| AMBP prot. IAT ILC | P02775 | AFIQLWAFDAVK | 113 | B | 563.6 | 457.3 | 46 | 200244 | 30431 | 15.2 | 0.97 | 0.04 | 4.1 | | | |
| AMBP prot. IAT ILC | P02774 | AFIQLWAFDAVK | 113 | b3 | 845.0 | 472.3 | 60 | 110462 | 12631 | 11.4 | 1.21 | 0.03 | 2.5 | | | |
| AMBP prot. IAT ILC | P02775 | AFIQLWAFDAVK | 117 | Y | 566.3 | 476.3 | 46 | 391966 | 55053 | 14.0 | | | | | | |
| AMBP prot. IAT ILC | P02775 | AFIQLWAFDAVK | 117 | B | 566.3 | 604.4 | 46 | 247258 | 37195 | 15.0 | | | | | | |
| AMBP prot. IAT ILC | P02775 | AFIQLWAFDAVK | 117 | B | 566.3 | 461.3 | 46 | 205421 | 30374 | 14.8 | | | | | | |
| AMBP prot. IAT ILC | P02774 | AFIQLWAFDAVK | 117 | Y | 849.0 | 476.3 | 60 | 91458 | 9751 | 10.7 | | | | | | |
| AMBP prot. IAT ILC | P02771 | TVAACNLPIVR | 113 | b3 | 677.4 | 412.3 | 52 | 666535 | 41995 | 6.3 | 1.29 | 0.05 | 4.0 | 1.31 | 0.04 | 3.4 |
| AMBP prot. IAT ILC | P02770 | TVAACNLPIVR | 113 | y4 | 677.4 | 484.3 | 52 | 441636 | 30548 | 6.9 | 1.36 | 0.03 | 2.4 | | | |
| AMBP prot. IAT ILC | P02771 | TVAACNLPIVR | 113 | b4 | 677.4 | 483.3 | 52 | 293459 | 15251 | 5.2 | 1.32 | 0.07 | 5.0 | | | |
| AMBP prot. IAT ILC | P02770 | TVAACNLPIVR | 113 | b6 | 677.4 | 757.4 | 52 | 140175 | 14268 | 10.2 | 1.25 | 0.06 | 4.9 | | | |
| AMBP prot. IAT ILC | P02771 | TVAACNLPIVR | 117 | B | 679.4 | 416.3 | 52 | 514837 | 26437 | 5.1 | | | | | | |
| AMBP prot. IAT ILC | P02770 | TVAACNLPIVR | 117 | B | 679.4 | 484.3 | 52 | 324144 | 18631 | 5.7 | | | | | | |
| AMBP prot. IAT ILC | P02771 | TVAACNLPIVR | 117 | B | 679.4 | 487.3 | 52 | 222815 | 13036 | 5.9 | | | | | | |
| AMBP prot. IAT ILC | P02770 | TVAACNLPIVR | 117 | B | 679.4 | 761.4 | 52 | 111698 | 9624 | 8.6 | | | | | | |
| Angiotensinogen | P01019 | ALQDQLVLVAAK | 113 | b4 | 517.0 | 568.3 | 44 | 564951 | 112480 | 19.9 | 0.97 | 0.05 | 5.2 | 0.99 | 0.03 | 3.0 |
| Angiotensinogen | P01019 | ALQDQLVLVAAK | 113 | b3 | 517.0 | 453.3 | 44 | 539688 | 103788 | 19.2 | 1.02 | 0.04 | 4.0 | | | |
| Angiotensinogen | P01019 | ALQDQLVLVAAK | 113 | b5 | 517.0 | 696.4 | 44 | 317365 | 60154 | 19.0 | 0.98 | 0.04 | 3.9 | | | |
| Angiotensinogen | P01019 | ALQDQLVLVAAK | 113 | Y5 | 517.0 | 641.4 | 44 | 209965 | 42912 | 20.4 | 4.39 | 0.51 | 11.6 | | | |
| Angiotensinogen | P01019 | ALQDQLVLVAAK | 117 | b4 | 519.7 | 572.3 | 44 | 582315 | 103319 | 17.7 | | | | | | |
| Angiotensinogen | P01019 | ALQDQLVLVAAK | 117 | b3 | 519.7 | 457.3 | 44 | 528145 | 106601 | 20.2 | | | | | | |
| Angiotensinogen | P01019 | ALQDQLVLVAAK | 117 | b5 | 519.7 | 700.4 | 44 | 325116 | 64331 | 19.8 | | | | | | |
| Angiotensinogen | P01019 | ALQDQLVLVAAK | 117 | Y5 | 519.7 | 645.4 | 44 | 48569 | 11586 | 23.9 | | | | | | |
| Angiotensinogen | P01019 | QPFVQGLALYTPWLPR | 113 | b3 | 680.1 | 513.3 | 52 | 629235 | 20776 | 3.3 | 1.08 | 0.06 | 5.6 | 1.03 | 0.06 | 5.6 |
| Angiotensinogen | P01019 | QPFVQGLALYTPWLPR | 113 | y4 | 680.1 | 484.3 | 52 | 318008 | 19998 | 6.3 | 0.97 | 0.05 | 5.2 | | | |
| Angiotensinogen | P01019 | QPFVQGLALYTPWLPR | 113 | b6 | 680.1 | 797.4 | 52 | 360775 | 24967 | 6.9 | 1.08 | 0.04 | 3.9 | | | |
| Angiotensinogen | P01019 | QPFVQGLALYTPWLPR | 113 | b4 | 680.1 | 612.4 | 52 | 424973 | 27315 | 6.4 | 0.99 | 0.03 | 3.2 | | | |
| Angiotensinogen | P01019 | QPFVQGLALYTPWLPR | 117 | b3 | 681.4 | 517.3 | 52 | 581897 | 33259 | 5.7 | | | | | | |
| Angiotensinogen | P01019 | QPFVQGLALYTPWLPR | 117 | y4 | 681.4 | 484.3 | 52 | 326997 | 16063 | 4.9 | | | | | | |
| Angiotensinogen | P01019 | QPFVQGLALYTPWLPR | 117 | b6 | 681.4 | 801.4 | 52 | 334544 | 17179 | 5.1 | | | | | | |
| Angiotensinogen | P01019 | QPFVQGLALYTPWLPR | 117 | b4 | 681.4 | 616.4 | 52 | 428471 | 21041 | 4.9 | | | | | | |
| Angiotensinogen | P01019 | SLDFTELDVAAEK | 113 | b3 | 573.3 | 456.2 | 47 | 1088455 | 144484 | 13.3 | 0.89 | 0.03 | 2.9 | 0.89 | 0.02 | 2.3 |
| Angiotensinogen | P01019 | SLDFTELDVAAEK | 113 | y3 | 573.3 | 487.3 | 47 | 247689 | 31285 | 12.6 | 0.88 | 0.08 | 8.8 | | | |
| Angiotensinogen | P01019 | SLDFTELDVAAEK | 113 | b4 | 573.3 | 603.3 | 47 | 117618 | 14496 | 12.3 | 0.92 | 0.08 | 8.6 | | | |
| Angiotensinogen | P01019 | SLDFTELDVAAEK | 113 | b6 | 573.3 | 833.4 | 47 | 40493 | 5093 | 12.6 | 0.87 | 0.07 | 7.9 | | | |
| Angiotensinogen | P01019 | SLDFTELDVAAEK | 117 | b3 | 576.0 | 460.3 | 47 | 1220279 | 159253 | 13.1 | | | | | | |
| Angiotensinogen | P01019 | SLDFTELDVAAEK | 117 | y3 | 576.0 | 491.3 | 47 | 283199 | 35408 | 12.5 | | | | | | |
| Angiotensinogen | P01019 | SLDFTELDVAAEK | 117 | b4 | 576.0 | 607.3 | 47 | 129224 | 18320 | 14.2 | | | | | | |
| Angiotensinogen | P01019 | SLDFTELDVAAEK | 117 | b6 | 576.0 | 837.4 | 47 | 46834 | 7425 | 15.9 | | | | | | |
| Antithrombin-III | P01008 | EVPLNTIIFMGR | 113 | y10 | 765.4 | 1161.6 | 56 | 220925 | 39897 | 18.1 | 0.96 | 0.10 | 10.8 | 0.93 | 0.04 | 4.5 |
| Antithrombin-III | P01008 | EVPLNTIIFMGR | 113 | y6 | 765.4 | 736.4 | 56 | 91611 | 17419 | 19.0 | 0.89 | 0.06 | 7.1 | | | |
| Antithrombin-III | P01008 | EVPLNTIIFMGR | 113 | y7 | 765.4 | 837.5 | 56 | 51454 | 8783 | 17.1 | 0.90 | 0.08 | 9.4 | | | |
| Antithrombin-III | P01008 | EVPLNTIIFMGR | 113 | y8 | 765.4 | 951.5 | 56 | 61262 | 18203 | 29.7 | 0.97 | 0.12 | 12.3 | | | |
| Antithrombin-III | P01008 | EVPLNTIIFMGR | 117 | y10 | 767.4 | 1161.6 | 56 | 232743 | 53085 | 22.8 | | | | | | |
| Antithrombin-III | P01008 | EVPLNTIIFMGR | 117 | y6 | 767.4 | 736.4 | 56 | 103503 | 23410 | 22.6 | | | | | | |
| Antithrombin-III | P01008 | EVPLNTIIFMGR | 117 | y7 | 767.4 | 837.5 | 56 | 58161 | 14089 | 24.2 | | | | | | |
| Antithrombin-III | P01008 | EVPLNTIIFMGR | 117 | y8 | 767.4 | 951.5 | 56 | 62471 | 14710 | 23.5 | | | | | | |
| Antithrombin-III | P01008 | LQPLDFK | 113 | y3 | 570.8 | 549.3 | 47 | 117985 | 28590 | 24.2 | 0.91 | 0.06 | 7.0 | 0.90 | 0.01 | 1.0 |
| Antithrombin-III | P01008 | LQPLDFK | 113 | y5 | 570.8 | 759.4 | 47 | 88464 | 18458 | 20.9 | 0.91 | 0.07 | 8.1 | | | |
| Antithrombin-III | P01008 | LQPLDFK | 113 | y4 | 570.8 | 662.4 | 47 | 67790 | 15405 | 22.7 | 0.89 | 0.09 | 9.7 | | | |
| Antithrombin-III | P01008 | LQPLDFK | 113 | b3 | 570.8 | 479.3 | 47 | | | | | | | | | |
| Antithrombin-III | P01008 | LQPLDFK | 117 | y3 | 574.8 | 553.3 | 47 | 134523 | 36649 | 27.2 | | | | | | |
| Antithrombin-III | P01008 | LQPLDFK | 117 | y5 | 574.8 | 763.4 | 47 | 97987 | 20834 | 21.3 | | | | | | |
| Antithrombin-III | P01008 | LQPLDFK | 117 | y4 | 574.8 | 666.4 | 47 | 77006 | 18397 | 23.9 | | | | | | |
| Antithrombin-III | P01008 | LQPLDFK | 117 | b3 | 574.8 | 483.3 | 47 | | | | | | | | | |
| Antithrombin-III | P01008 | TSDQIHFFFAK | 113 | b3 | 541.0 | 444.2 | 45 | 1183129 | 236973 | 20.0 | 1.42 | 0.04 | 2.7 | 1.32 | 0.09 | 6.7 |
| Antithrombin-III | P01008 | TSDQIHFFFAK | 113 | b4 | 541.0 | 572.3 | 45 | 545183 | 110276 | 20.2 | 1.25 | 0.03 | 2.5 | | | |
| Antithrombin-III | P01008 | TSDQIHFFFAK | 113 | y3 | 541.0 | 505.3 | 45 | 188674 | 39020 | 20.7 | 1.24 | 0.08 | 6.1 | | | |
| Antithrombin-III | P01008 | TSDQIHFFFAK | 113 | y4 | 541.0 | 652.4 | 45 | 66938 | 13192 | 19.7 | 1.38 | 0.19 | 13.9 | | | |
| Antithrombin-III | P01008 | TSDQIHFFFAK | 117 | b3 | 543.6 | 448.2 | 45 | 835367 | 165166 | 19.8 | | | | | | |
| Antithrombin-III | P01008 | TSDQIHFFFAK | 117 | b4 | 543.6 | 576.3 | 45 | 434879 | 86806 | 20.0 | | | | | | |
| Antithrombin-III | P01008 | TSDQIHFFFAK | 117 | y3 | 543.6 | 509.3 | 45 | 151638 | 27266 | 18.0 | | | | | | |
| Antithrombin-III | P01008 | TSDQIHFFFAK | 117 | y4 | 543.6 | 656.4 | 45 | 49036 | 9773 | 19.9 | | | | | | |
| Approt. A-I | P02647 | DLATVYVDVLK | 113 | Y5 | 758.4 | 713.5 | 56 | 1881140 | 897807 | 47.7 | 1.14 | 0.09 | 7.9 | 1.23 | 0.06 | 5.2 |
| Approt. A-I | P02647 | DLATVYVDVLK | 113 | Y6 | 758.4 | 876.5 | 56 | 1268173 | 585729 | 46.2 | 1.26 | 0.11 | 9.1 | | | |
| Approt. A-I | P02647 | DLATVYVDVLK | 113 | B6 | 758.4 | 803.4 | 56 | 905527 | 418479 | 46.2 | 1.29 | 0.10 | 7.6 | | | |
| Approt. A-I | P02647 | DLATVYVDVLK | 113 | y4 | 758.4 | 614.4 | 56 | 3954419 | 1839794 | 46.5 | 1.23 | 0.06 | 5.0 | | | |
| Approt. A-I | P02647 | DLATVYVDVLK | 117 | Y5 | 762.4 | 717.5 | 56 | 1624487 | 752057 | 46.3 | | | | | | |
| Approt. A-I | P02647 | DLATVYVDVLK | 117 | Y6 | 762.4 | 880.5 | 56 | 1011288 | 498831 | 49.3 | | | | | | |
| Approt. A-I | P02647 | DLATVYVDVLK | 117 | B6 | 762.4 | 807.4 | 56 | 696327 | 318314 | 45.7 | | | | | | |
| Approt. A-I | P02647 | DLATVYVDVLK | 117 | y4 | 762.4 | 618.4 | 56 | 3183263 | 1430406 | 44.9 | | | | | | |
| Approt. A-I | P02647 | DYVSQFEGSALGK | 113 | y5 | 560.9 | 615.4 | 46 | 578567 | 84127 | 14.5 | 0.92 | 0.02 | 2.5 | 0.95 | 0.03 | 2.8 |
| Approt. A-I | P02647 | DYVSQFEGSALGK | 113 | y6 | 560.9 | 672.4 | 46 | 1240323 | 181105 | 14.6 | 0.95 | 0.03 | 3.5 | | | |
| Approt. A-I | P02647 | DYVSQFEGSALGK | 113 | y6 | 840.9 | 672.4 | 60 | 3774577 | 361454 | 9.6 | 0.99 | 0.03 | 2.6 | | | |
| Approt. A-I | P02647 | DYVSQFEGSALGK | 113 | b5 | 560.9 | 733.4 | 46 | 1782165 | 245652 | 13.8 | 0.95 | 0.03 | 3.3 | | | |
| Approt. A-I | P02647 | DYVSQFEGSALGK | 117 | y5 | 563.6 | 619.4 | 46 | 627734 | 88169 | 14.0 | | | | | | |
| Approt. A-I | P02647 | DYVSQFEGSALGK | 117 | y6 | 563.6 | 676.4 | 46 | 1301824 | 176823 | 13.6 | | | | | | |
| Approt. A-I | P02647 | DYVSQFEGSALGK | 117 | y6 | 844.9 | 676.4 | 60 | 3829251 | 378683 | 9.9 | | | | | | |
| Approt. A-I | P02647 | DYVSQFEGSALGK | 117 | b5 | 563.6 | 737.4 | 46 | 1879769 | 234709 | 12.5 | | | | | | |
| Approt. A-I | P02647 | LLDNWDSVTSTFSK | 113 | Y8 | 947.0 | 996.5 | 65 | 725972 | 139997 | 19.3 | 1.13 | 0.07 | 6.1 | 1.08 | 0.07 | 6.1 |
| Approt. A-I | P02647 | LLDNWDSVTSTFSK | 113 | B7 | 947.0 | 984.5 | 65 | 395657 | 79846 | 20.2 | 1.14 | 0.05 | 4.0 | | | |
| Approt. A-I | P02647 | LLDNWDSVTSTFSK | 113 | Y9 | 947.0 | 1111.6 | 65 | 480746 | 87726 | 18.2 | 1.00 | 0.07 | 6.7 | | | |
| Approt. A-I | P02647 | LLDNWDSVTSTFSK | 113 | B4 | 947.0 | 596.3 | 65 | 3713511 | 741712 | 20.0 | 1.07 | 0.03 | 2.6 | | | |
| Approt. A-I | P02647 | LLDNWDSVTSTFSK | 117 | Y8 | 951.0 | 1000.5 | 66 | 645205 | 129291 | 20.0 | | | | | | |
| Approt. A-I | P02647 | LLDNWDSVTSTFSK | 117 | B7 | 951.0 | 988.5 | 66 | 348135 | 68045 | 19.5 | | | | | | |
| Approt. A-I | P02647 | LLDNWDSVTSTFSK | 117 | Y9 | 951.0 | 1115.6 | 66 | 486285 | 102140 | 21.0 | | | | | | |
| Approt. A-I | P02647 | LLDNWDSVTSTFSK | 117 | B4 | 951.0 | 600.3 | 66 | 3478946 | 653684 | 18.8 | | | | | | |
| Approt. A-I | P02647 | LSPLGEEMR | 113 | y5 | 586.3 | 621.3 | 47 | 1919500 | 278953 | 14.5 | 0.95 | 0.02 | 2.4 | 0.98 | 0.03 | 3.2 |
| Approt. A-I | P02647 | LSPLGEEMR | 113 | y6 | 586.3 | 734.3 | 47 | 1254755 | 194055 | 15.5 | 0.98 | 0.03 | 2.7 | | | |
| Approt. A-I | P02647 | LSPLGEEMR | 113 | y6 | 586.3 | 918.4 | 47 | 1464076 | 224675 | 15.3 | 0.97 | 0.02 | 1.9 | | | |
| Approt. A-I | P02647 | LSPLGEEMR | 113 | b5 | 586.3 | 608.4 | 47 | 899502 | 127836 | 14.2 | 1.02 | 0.03 | 3.0 | | | |
| Approt. A-I | P02647 | LSPLGEEMR | 117 | y5 | 588.3 | 621.3 | 47 | 2021083 | 313142 | 15.5 | | | | | | |
| Approt. A-I | P02647 | LSPLGEEMR | 117 | y6 | 588.3 | 734.3 | 47 | 1285833 | 212648 | 16.5 | | | | | | |
| Approt. A-I | P02647 | LSPLGEEMR | 117 | y8 | 588.3 | 918.4 | 47 | 1514862 | 245516 | 16.2 | | | | | | |
| Approt. A-I | P02647 | LSPLGEEMR | 117 | b5 | 588.3 | 612.4 | 47 | 879893 | 133536 | 15.2 | | | | | | |
| Approt. A-I | P02647 | VQPYLDDFQK | 113 | y3 | 766.9 | 562.3 | 56 | 4036738 | 1163545 | 28.8 | 1.40 | 0.05 | 3.4 | 1.44 | 0.12 | 8.2 |
| Approt. A-I | P02647 | VQPYLDDFQK | 113 | y4 | 766.9 | 677.4 | 56 | 2153229 | 612839 | 28.5 | 1.37 | 0.08 | 6.0 | | | |
| Approt. A-I | P02647 | VQPYLDDFQK | 113 | y5 | 766.9 | 792.4 | 56 | 1150582 | 313751 | 27.3 | 1.36 | 0.04 | 3.2 | | | |
| Approt. A-I | P02647 | VQPYLDDFQK | 113 | y8 | 766.9 | 1165.6 | 56 | 649263 | 137234 | 21.1 | 1.61 | 0.10 | 6.0 | | | |
| Approt. A-I | P02647 | VQPYLDDFQK | 117 | y3 | 770.9 | 566.3 | 57 | 2861723 | 775269 | 27.1 | | | | | | |
| Approt. A-I | P02647 | VQPYLDDFQK | 117 | y4 | 770.9 | 681.4 | 57 | 1553922 | 406060 | 26.1 | | | | | | |
| Approt. A-I | P02647 | VQPYLDDFQK | 117 | y5 | 770.9 | 796.4 | 57 | 845654 | 224488 | 26.5 | | | | | | |
| Approt. A-I | P02647 | VQPYLDDFQK | 117 | y8 | 770.9 | 1169.6 | 57 | 402320 | 75158 | 18.7 | | | | | | |
| approt. A-II P† | P02652 | AGTELVNFLSYFVELGTQPATQ | 113 | b4 | 842.4 | 499.3 | 60 | 87448 | 6202 | 7.1 | 0.90 | 0.05 | 5.1 | 0.98 | 0.07 | 7.0 |
| approt. A-II P† | P02652 | AGTELVNFLSYFVELGTQPATQ | 113 | b5 | 842.4 | 612.3 | 60 | 71952 | 4358 | 6.1 | 0.95 | 0.04 | 4.6 | | | |
| approt. A-II P† | P02652 | AGTELVNFLSYFVELGTQPATQ | 113 | b7 | 842.4 | 825.5 | 60 | 50039 | 4674 | 9.3 | 1.06 | 0.07 | 6.8 | | | |
| approt. A-II P† | P02652 | AGTELVNFLSYFVELGTQPATQ | 113 | b4 | 842.4 | 711.4 | 60 | 53333 | 3919 | 7.3 | 0.99 | 0.04 | 4.0 | | | |
| approt. A-II P† | P02652 | AGTELVNFLSYFVELGTQPATQ | 117 | b4 | 843.8 | 503.3 | 60 | 97154 | 4184 | 4.3 | | | | | | |
| approt. A-II P† | P02652 | AGTELVNFLSYFVELGTQPATQ | 117 | b5 | 843.8 | 616.3 | 60 | 75518 | 5502 | 7.3 | | | | | | |
| approt. A-II P† | P02652 | AGTELVNFLSYFVELGTQPATQ | 117 | b7 | 843.8 | 829.5 | 60 | 47252 | 5144 | 10.9 | | | | | | |
| approt. A-II P† | P02652 | AGTELVNFLSYFVELGTQPATQ | 117 | b4 | 843.8 | 715.4 | 60 | 53995 | 4103 | 7.6 | | | | | | |
| approt. A-II P† | P02652 | EQLTPLIK | 113 | y4 | 611.4 | 610.4 | 49 | 1115008 | 292690 | 26.3 | 0.96 | 0.07 | 6.9 | 0.95 | 0.01 | 1.2 |
| approt. A-II P† | P02652 | EQLTPLIK | 113 | b3 | 611.4 | 511.3 | 49 | 769969 | 207941 | 27.0 | 0.95 | 0.05 | 5.5 | | | |
| approt. A-II P† | P02652 | EQLTPLIK | 113 | y3 | 611.4 | 513.4 | 49 | 477077 | 123059 | 25.8 | 0.96 | 0.07 | 7.1 | | | |
| approt. A-II P† | P02652 | EQLTPLIK | 113 | y5 | 611.4 | 711.5 | 49 | 154079 | 43117 | 28.0 | 0.94 | 0.07 | 7.9 | | | |
| approt. A-II P† | P02652 | EQLTPLIK | 117 | y4 | 615.4 | 614.4 | 49 | 1176940 | 340097 | 28.9 | | | | | | |
| approt. A-II P† | P02652 | EQLTPLIK | 117 | b3 | 615.4 | 515.3 | 49 | 812209 | 241787 | 29.8 | | | | | | |
| approt. A-II P† | P02652 | EQLTPLIK | 117 | y3 | 615.4 | 517.4 | 49 | 499220 | 141256 | 28.3 | | | | | | |
| approt. A-II P† | P02652 | EQLTPLIK | 117 | y5 | 615.4 | 715.5 | 49 | 165290 | 48747 | 29.5 | | | | | | |
| approt. A-II P† | P02652 | SPELQAEAK | 113 | b3 | 626.9 | 454.2 | 49 | 330413 | 103956 | 31.5 | 1.07 | 0.09 | 8.0 | 1.10 | 0.07 | 6.6 |
| approt. A-II P† | P02652 | SPELQAEAK | 113 | y4 | 626.9 | 558.3 | 49 | 325392 | 100934 | 31.0 | 1.16 | 0.12 | 10.7 | | | |
| approt. A-II P† | P02652 | SPELQAEAK | 113 | Y6 | 626.9 | 799.5 | 49 | 84820 | 31336 | 36.9 | 1.18 | 0.11 | 9.3 | | | |
| approt. A-II P† | P02652 | SPELQAEAK | 113 | Y3 | 626.9 | 487.3 | 49 | 284803 | 84859 | 29.8 | 1.02 | 0.06 | 5.7 | | | |
| approt. A-II P† | P02652 | SPELQAEAK | 117 | b3 | 630.9 | 458.2 | 50 | 310030 | 101213 | 32.6 | | | | | | |
| approt. A-II P† | P02652 | SPELQAEAK | 117 | y4 | 630.9 | 562.3 | 50 | 284713 | 94728 | 33.3 | | | | | | |
| approt. A-II P† | P02652 | SPELQAEAK | 117 | Y6 | 630.9 | 803.5 | 50 | 72287 | 25733 | 35.6 | | | | | | |
| approt. A-II P† | P02652 | SPELQAEAK | 117 | Y3 | 630.9 | 491.3 | 50 | 279007 | 77173 | 27.7 | | | | | | |
| Approt. A-IV | P06727 | ALVQQMEQLR | 113 | y7 | 678.4 | 932.5 | 52 | 95458 | 15183 | 15.9 | 0.95 | 0.05 | 4.9 | 0.88 | 0.06 | 7.2 |
| Approt. A-IV | P06727 | ALVQQMEQLR | 113 | b3 | 678.4 | 424.3 | 52 | 404339 | 97683 | 24.2 | 0.80 | 0.02 | 3.1 | | | |
| Approt. A-IV | P06727 | ALVQQMEQLR | 113 | y5 | 678.4 | 676.3 | 52 | 103593 | 19766 | 19.1 | 0.89 | 0.06 | 7.2 | | | |
| Approt. A-IV | P06727 | ALVQQMEQLR | 113 | y4 | 678.4 | 545.3 | 52 | 87113 | 20363 | 23.4 | 0.89 | 0.05 | 5.8 | | | |
| Approt. A-IV | P06727 | ALVQQMEQLR | 117 | y7 | 680.4 | 932.5 | 52 | 100362 | 15344 | 15.3 | | | | | | |
| Approt. A-IV | P06727 | ALVQQMEQLR | 117 | b3 | 680.4 | 428.3 | 52 | 505200 | 120467 | 23.8 | | | | | | |
| Approt. A-IV | P06727 | ALVQQMEQLR | 117 | y5 | 680.4 | 676.3 | 52 | 116623 | 23474 | 20.1 | | | | | | |
| Approt. A-IV | P06727 | ALVQQMEQLR | 117 | y4 | 680.4 | 545.3 | 52 | 97353 | 21127 | 21.7 | | | | | | |
| Approt. A-IV | P06727 | ISASAEELR | 113 | y8 | 558.3 | 862.4 | 46 | 152454 | 39477 | 25.9 | 1.08 | 0.06 | 5.7 | 1.06 | 0.01 | 1.4 |
| Approt. A-IV | P06727 | ISASAEELR | 113 | y3 | 558.3 | 417.2 | 46 | 334660 | 43125 | 12.9 | 1.05 | 0.05 | 4.5 | | | |
| Approt. A-IV | P06727 | ISASAEELR | 113 | y6 | 558.3 | 704.4 | 46 | 63008 | 12279 | 19.5 | 1.04 | 0.07 | 7.2 | | | |
| Approt. A-IV | P06727 | ISASAEELR | 113 | y5 | 558.3 | 617.3 | 46 | 49486 | 7700 | 15.6 | 1.06 | 0.08 | 7.1 | | | |
| Approt. A-IV | P06727 | ISASAEELR | 117 | y8 | 560.3 | 862.4 | 46 | 141200 | 34857 | 24.7 | | | | | | |
| Approt. A-IV | P06727 | ISASAEELR | 117 | y3 | 560.3 | 417.2 | 46 | 319871 | 46841 | 14.6 | | | | | | |
| Approt. A-IV | P06727 | ISASAEELR | 117 | y6 | 560.3 | 704.4 | 46 | 60447 | 12183 | 20.2 | | | | | | |
| Approt. A-IV | P06727 | ISASAEELR | 117 | y5 | 560.3 | 617.3 | 46 | 46513 | 6232 | 13.4 | | | | | | |
| Approt. A-IV | P06727 | LEPYADQLR | 113 | y3 | 622.8 | 416.3 | 49 | 1051530 | 212214 | 20.2 | 0.91 | 0.05 | 5.1 | 1.03 | 0.11 | 10.7 |
| Approt. A-IV | P06727 | LEPYADQLR | 113 | y7 | 622.8 | 862.4 | 49 | 398700 | 72728 | 18.2 | 1.12 | 0.06 | 5.2 | | | |
| Approt. A-IV | P06727 | LEPYADQLR | 113 | b3 | 622.8 | 480.3 | 49 | 184942 | 31098 | 16.8 | 0.96 | 0.07 | 7.3 | | | |
| Approt. A-IV | P06727 | LEPYADQLR | 113 | y6 | 622.8 | 765.4 | 49 | 107445 | 18463 | 17.2 | 1.12 | 0.08 | 6.8 | | | |
| Approt. A-IV | P06727 | LEPYADQLR | 117 | y3 | 624.8 | 416.3 | 49 | 1155313 | 203900 | 17.6 | | | | | | |
| Approt. A-IV | P06727 | LEPYADQLR | 117 | y7 | 624.8 | 862.4 | 49 | 355392 | 61961 | 17.4 | | | | | | |
| Approt. A-IV | P06727 | LEPYADQLR | 117 | b3 | 624.8 | 484.3 | 49 | 192082 | 29120 | 15.2 | | | | | | |
| Approt. A-IV | P06727 | LEPYADQLR | 117 | y6 | 624.8 | 765.4 | 49 | 95726 | 14778 | 15.4 | | | | | | |
| Approt. A-IV | P06727 | LGEVNTYAGDLQK | 113 | b4 | 563.3 | 539.3 | 46 | 529683 | 36847 | 7.0 | 1.03 | 0.05 | 4.7 | 0.99 | 0.05 | 5.0 |
| Approt. A-IV | P06727 | LGEVNTYAGDLQK | 113 | b5 | 563.3 | 653.4 | 46 | 534761 | 42766 | 8.0 | 1.00 | 0.04 | 4.4 | | | |
| Approt. A-IV | P06727 | LGEVNTYAGDLQK | 113 | y3 | 563.3 | 528.4 | 46 | 263332 | 26395 | 10.0 | 1.00 | 0.03 | 3.3 | | | |
| Approt. A-IV | P06727 | LGEVNTYAGDLQK | 113 | b6 | 563.3 | 754.4 | 46 | 63938 | 7712 | 12.1 | 0.92 | 0.08 | 8.3 | | | |
| Approt. A-IV | P06727 | LGEVNTYAGDLQK | 117 | b4 | 566.0 | 543.3 | 46 | 514323 | 37749 | 7.3 | | | | | | |
| Approt. A-IV | P06727 | LGEVNTYAGDLQK | 117 | b5 | 566.0 | 657.4 | 46 | 535684 | 44305 | 8.3 | | | | | | |
| Approt. A-IV | P06727 | LGEVNTYAGDLQK | 117 | y3 | 566.0 | 532.4 | 46 | 263197 | 27394 | 10.4 | | | | | | |
| Approt. A-IV | P06727 | LGEVNTYAGDLQK | 117 | b6 | 566.0 | 758.4 | 46 | 70144 | 10202 | 14.5 | | | | | | |
| Approt. A-IV | P06727 | SELTQQLNALFQDK | 113 | y4 | 639.0 | 677.4 | 50 | 458059 | 67951 | 14.8 | 1.14 | 0.08 | 6.7 | 1.24 | 0.12 | 9.7 |
| Approt. A-IV | P06727 | SELTQQLNALFQDK | 113 | b3 | 639.0 | 470.3 | 50 | 1942709 | 395199 | 20.3 | 1.25 | 0.09 | 6.9 | | | |
| Approt. A-IV | P06727 | SELTQQLNALFQDK | 113 | y6 | 639.0 | 827.4 | 50 | 197893 | 31050 | 15.7 | 1.18 | 0.07 | 6.3 | | | |
| Approt. A-IV | P06727 | SELTQQLNALFQDK | 113 | y5 | 639.0 | 790.5 | 50 | 105472 | 14484 | 13.7 | 1.41 | 0.18 | 12.8 | | | |
| Approt. A-IV | P06727 | SELTQQLNALFQDK | 117 | y4 | 641.7 | 681.4 | 50 | 404965 | 63705 | 15.7 | | | | | | |
| Approt. A-IV | P06727 | SELTQQLNALFQDK | 117 | b3 | 641.7 | 474.3 | 50 | 1554134 | 293615 | 18.9 | | | | | | |
| Approt. A-IV | P06727 | SELTQQLNALFQDK | 117 | y6 | 641.7 | 831.4 | 50 | 167557 | 23768 | 14.2 | | | | | | |
| Approt. A-IV | P06727 | SELTQQLNALFQDK | 117 | y5 | 641.7 | 794.5 | 50 | 75390 | 12120 | 16.1 | | | | | | |
| Approt. A-IV | P06727 | TQVNTQAEQLR | 113 | y8 | 714.4 | 959.5 | 54 | 42710 | 11724 | 27.4 | 1.08 | 0.14 | 12.6 | 1.25 | 0.12 | 9.3 |
| Approt. A-IV | P06727 | TQVNTQAEQLR | 113 | y10 | 714.4 | 1186.6 | 54 | 22604 | 5433 | 24.0 | 1.32 | 0.11 | 8.5 | | | |
| Approt. A-IV | P06727 | TQVNTQAEQLR | 113 | y7 | 714.4 | 845.4 | 54 | 41147 | 10583 | 25.7 | 1.27 | 0.12 | 9.6 | | | |
| Approt. A-IV | P06727 | TQVNTQAEQLR | 113 | y9 | 714.4 | 1058.6 | 54 | 27790 | 7601 | 27.4 | 1.32 | 0.18 | 13.8 | | | |
| Approt. A-IV | P06727 | TQVNTQAEQLR | 117 | y8 | 716.4 | 959.5 | 54 | 39297 | 7606 | 19.4 | | | | | | |
| Approt. A-IV | P06727 | TQVNTQAEQLR | 117 | y10 | 716.4 | 1186.6 | 54 | 17280 | 4923 | 28.5 | | | | | | |
| Approt. A-IV | P06727 | TQVNTQAEQLR | 117 | y7 | 716.4 | 845.4 | 54 | 32479 | 7516 | 23.1 | | | | | | |
| Approt. A-IV | P06727 | TQVNTQAEQLR | 117 | y9 | 716.4 | 1058.6 | 54 | 21024 | 4909 | 23.4 | | | | | | |
| Approt. B-100 | P04114 | NNALDFVTK | 113 | b3 | 651.4 | 440.2 | 51 | 242825 | 35177 | 14.5 | 1.19 | 0.05 | 4.3 | 1.14 | 0.04 | 3.1 |
| Approt. B-100 | P04114 | NNALDFVTK | 113 | y3 | 651.4 | 487.3 | 51 | 84920 | 11620 | 13.7 | 1.14 | 0.12 | 10.3 | | | |
| Approt. B-100 | P04114 | NNALDFVTK | 113 | b5 | 651.4 | 668.3 | 51 | 127756 | 12649 | 9.9 | 1.11 | 0.07 | 5.9 | | | |
| Approt. B-100 | P04114 | NNALDFVTK | 113 | y4 | 651.4 | 634.4 | 51 | 91622 | 11402 | 12.4 | 1.14 | 0.10 | 9.0 | | | |
| Approt. B-100 | P04114 | NNALDFVTK | 117 | b3 | 655.4 | 444.2 | 51 | 203514 | 28520 | 14.0 | | | | | | |
| Approt. B-100 | P04114 | NNALDFVTK | 117 | y3 | 655.4 | 491.3 | 51 | 75225 | 11335 | 15.1 | | | | | | |
| Approt. B-100 | P04114 | NNALDFVTK | 117 | b5 | 655.4 | 672.3 | 51 | 115695 | 14661 | 12.7 | | | | | | |
| Approt. B-100 | P04114 | NNALDFVTK | 117 | y4 | 655.4 | 638.4 | 51 | 80848 | 9704 | 12.0 | | | | | | |
| Approt. B-100 | P04114 | SVSLPSLDPASAK | 113 | b3 | 518.0 | 414.2 | 44 | 473381 | 44540 | 9.4 | 0.98 | 0.03 | 3.2 | 0.99 | 0.06 | 6.1 |
| Approt. B-100 | P04114 | SVSLPSLDPASAK | 113 | y3 | 518.0 | 445.3 | 44 | 371108 | 40776 | 11.0 | 1.06 | 0.03 | 3.3 | | | |
| Approt. B-100 | P04114 | SVSLPSLDPASAK | 113 | y5 | 518.0 | 613.4 | 44 | 81530 | 9556 | 11.7 | 1.01 | 0.05 | 5.0 | | | |
| Approt. B-100 | P04114 | SVSLPSLDPASAK | 113 | b4 | 518.0 | 527.3 | 44 | 110736 | 16436 | 14.8 | 0.91 | 0.06 | 6.7 | | | |
| Approt. B-100 | P04114 | SVSLPSLDPASAK | 117 | b3 | 520.6 | 418.2 | 44 | 481898 | 49770 | 10.3 | | | | | | |
| Approt. B-100 | P04114 | SVSLPSLDPASAK | 117 | y3 | 520.6 | 449.3 | 44 | 350505 | 33821 | 9.6 | | | | | | |
| Approt. B-100 | P04114 | SVSLPSLDPASAK | 117 | y5 | 520.6 | 617.4 | 44 | 81049 | 10009 | 12.3 | | | | | | |
| Approt. B-100 | P04114 | SVSLPSLDPASAK | 117 | b4 | 520.6 | 531.3 | 44 | 121138 | 13742 | 11.3 | | | | | | |
| Approt. B-100 | P04114 | YGMVAQVTQTLK | 113 | b3 | 540.3 | 492.2 | 45 | 544781 | 93764 | 17.2 | 1.24 | 0.04 | 3.4 | 1.30 | 0.08 | 6.2 |
| Approt. B-100 | P04114 | YGMVAQVTQTLK | 113 | b4 | 540.3 | 591.3 | 45 | 248098 | 38910 | 15.7 | 1.37 | 0.04 | 3.0 | | | |
| Approt. B-100 | P04114 | YGMVAQVTQTLK | 113 | y3 | 540.3 | 501.3 | 45 | 115960 | 19582 | 16.9 | 1.37 | 0.05 | 3.8 | | | |
| Approt. B-100 | P04114 | YGMVAQVTQTLK | 113 | y5 | 540.3 | 662.3 | 45 | 95707 | 14427 | 15.1 | 1.22 | 0.15 | 12.2 | | | |
| Approt. B-100 | P04114 | YGMVAQVTQTLK | 117 | b3 | 543.0 | 496.2 | 45 | 441087 | 80437 | 18.2 | | | | | | |
| Approt. B-100 | P04114 | YGMVAQVTQTLK | 117 | b4 | 543.0 | 595.3 | 45 | 181377 | 31180 | 17.2 | | | | | | |
| Approt. B-100 | P04114 | YGMVAQVTQTLK | 117 | y3 | 543.0 | 505.3 | 45 | 85117 | 16175 | 19.0 | | | | | | |
| Approt. B-100 | P04114 | YGMVAQVTQTLK | 117 | y5 | 543.0 | 666.3 | 45 | 79669 | 16315 | 20.5 | | | | | | |
| Approt. C-I | P02654 | EFGNTLEDK | 113 | b4 | 666.8 | 588.3 | 51 | 251166 | 26340 | 10.5 | 1.13 | 0.11 | 10.1 | 1.32 | 0.15 | 11.4 |
| Approt. C-I | P02654 | EFGNTLEDK | 113 | y3 | 666.8 | 531.3 | 51 | 362035 | 48616 | 13.4 | 1.49 | 0.12 | 8.3 | | | |
| Approt. C-I | P02654 | EFGNTLEDK | 113 | b3 | 666.8 | 474.2 | 51 | 228113 | 30379 | 13.3 | 1.32 | 0.15 | 11.2 | | | |
| Approt. C-I | P02654 | EFGNTLEDK | 113 | y4 | 666.8 | 644.4 | 51 | 168369 | 17088 | 10.1 | 1.33 | 0.12 | 8.7 | | | |
| Approt. C-I | P02654 | EFGNTLEDK | 117 | b4 | 670.8 | 592.3 | 52 | 225314 | 36986 | 16.4 | | | | | | |
| Approt. C-I | P02654 | EFGNTLEDK | 117 | y3 | 670.8 | 535.3 | 52 | 242350 | 25866 | 10.7 | | | | | | |
| Approt. C-I | P02654 | EFGNTLEDK | 117 | b3 | 670.8 | 478.2 | 52 | 174830 | 27738 | 15.9 | | | | | | |
| Approt. C-I | P02654 | EFGNTLEDK | 117 | y4 | 670.8 | 648.4 | 52 | 126906 | 13800 | 10.9 | | | | | | |
| Approt. C-I | P02654 | EWFSETFQK | 113 | y3 | 741.4 | 562.3 | 55 | 116044 | 9627 | 8.3 | 0.97 | 0.10 | 10.2 | 0.96 | 0.02 | 2.2 |
| Approt. C-I | P02654 | EWFSETFQK | 113 | b3 | 741.4 | 603.3 | 55 | 57745 | 5861 | 10.2 | 0.98 | 0.13 | 13.4 | | | |
| Approt. C-I | P02654 | EWFSETFQK | 113 | y6 | 741.4 | 879.5 | 55 | 45086 | 4560 | 10.1 | 0.94 | 0.12 | 12.9 | | | |
| Approt. C-I | P02654 | EWFSETFQK | 113 | y7 | 741.4 | 1026.5 | 55 | 17970 | 1276 | 7.1 | 0.94 | 0.13 | 14.3 | | | |
| Approt. C-I | P02654 | EWFSETFQK | 117 | y3 | 745.4 | 566.3 | 55 | 120652 | 11978 | 9.9 | | | | | | |
| Approt. C-I | P02654 | EWFSETFQK | 117 | b3 | 745.4 | 607.3 | 55 | 59499 | 9002 | 15.1 | | | | | | |
| Approt. C-I | P02654 | EWFSETFQK | 117 | y6 | 745.4 | 883.5 | 55 | 48507 | 5447 | 11.2 | | | | | | |
| Approt. C-I | P02654 | EWFSETFQK | 117 | y7 | 745.4 | 1030.5 | 55 | 19315 | 2436 | 12.6 | | | | | | |
| Approt. C-III | P02656 | DALSSVQESQVAQQAR | 113 | b4 | 619.7 | 527.3 | 49 | 1111256 | 120769 | 10.9 | 1.00 | 0.03 | 3.2 | 1.11 | 0.07 | 6.6 |
| Approt. C-III | P02656 | DALSSVQESQVAQQAR | 113 | b5 | 619.7 | 614.3 | 49 | 947909 | 115980 | 12.2 | 1.17 | 0.04 | 3.4 | | | |
| Approt. C-III | P02656 | DALSSVQESQVAQQAR | 113 | b3 | 929.0 | 440.3 | 64 | 2596081 | 318730 | 12.3 | 1.12 | 0.07 | 6.6 | | | |
| Approt. C-III | P02656 | DALSSVQESQVAQQAR | 113 | y8 | 929.0 | 887.5 | 64 | 584056 | 36615 | 6.3 | 1.14 | 0.15 | 13.4 | | | |
| Approt. C-III | P02656 | DALSSVQESQVAQQAR | 117 | b4 | 621.0 | 531.3 | 49 | 1110847 | 132204 | 11.9 | | | | | | |
| Approt. C-III | P02656 | DALSSVQESQVAQQAR | 117 | b5 | 621.0 | 618.3 | 49 | 813095 | 122434 | 15.1 | | | | | | |
| Approt. C-III | P02656 | DALSSVQESQVAQQAR | 117 | b3 | 931.0 | 444.3 | 65 | 2330030 | 399289 | 17.1 | | | | | | |
| Approt. C-III | P02656 | DALSSVQESQVAQQAR | 117 | y8 | 931.0 | 887.5 | 65 | 524575 | 94043 | 17.9 | | | | | | |
| Approt. C-III | P02656 | GWVTDGFSSLK | 113 | y3 | 738.9 | 487.3 | 55 | 179309 | 11770 | 6.6 | 0.80 | 0.04 | 5.4 | 0.88 | 0.05 | 6.1 |
| Approt. C-III | P02656 | GWVTDGFSSLK | 113 | y4 | 738.9 | 574.4 | 55 | 141263 | 13246 | 9.4 | 0.92 | 0.05 | 5.4 | | | |
| Approt. C-III | P02656 | GWVTDGFSSLK | 113 | y6 | 738.9 | 778.4 | 55 | 142655 | 9672 | 6.8 | 0.90 | 0.06 | 7.2 | | | |
| Approt. C-III | P02656 | GWVTDGFSSLK | 113 | b5 | 738.9 | 699.3 | 55 | 85990 | 7624 | 8.9 | 0.88 | 0.09 | 10.0 | | | |
| Approt. C-III | P02656 | GWVTDGFSSLK | 117 | y3 | 742.9 | 491.3 | 55 | 225143 | 19892 | 8.8 | | | | | | |
| Approt. C-III | P02656 | GWVTDGFSSLK | 117 | y4 | 742.9 | 578.4 | 55 | 153516 | 10895 | 7.1 | | | | | | |
| Approt. C-III | P02656 | GWVTDGFSSLK | 117 | y6 | 742.9 | 782.5 | 55 | 158809 | 9580 | 6.0 | | | | | | |
| Approt. C-III | P02656 | GWVTDGFSSLK | 117 | b5 | 742.9 | 703.4 | 55 | 97818 | 8284 | 8.5 | | | | | | |
| Approt. C-III | P02656 | SEAEDASLLSFMQGYMK | 113 | b7 | 729.7 | 830.4 | 54 | 696027 | 70592 | 10.1 | 1.20 | 0.02 | 2.0 | 1.13 | 0.06 | 5.6 |
| Approt. C-III | P02656 | SEAEDASLLSFMQGYMK | 113 | b6 | 729.7 | 743.3 | 54 | 1174816 | 132469 | 11.3 | 1.05 | 0.04 | 3.7 | | | |
| Approt. C-III | P02656 | SEAEDASLLSFMQGYMK | 113 | b5 | 729.7 | 672.3 | 54 | 2727076 | 372361 | 13.7 | 1.12 | 0.03 | 2.6 | | | |
| Approt. C-III | P02656 | SEAEDASLLSFMQGYMK | 113 | b5 | 1094.0 | 672.3 | 73 | 414946 | 50520 | 12.2 | 1.15 | 0.06 | 5.3 | | | |
| Approt. C-III | P02656 | SEAEDASLLSFMQGYMK | 3 | b7 | 732.4 | 834.4 | 55 | 580239 | 62355 | 10.7 | | | | | | |
| Approt. C-III | P02656 | SEAEDASLLSFMQGYMK | 3 | b6 | 732.4 | 747.3 | 55 | 1122929 | 142991 | 12.7 | | | | | | |
| Approt. C-III | P02656 | SEAEDASLLSFMQGYMK | 3 | b5 | 732.4 | 676.3 | 55 | 2428527 | 317026 | 13.1 | | | | | | |
| Approt. C-III | P02656 | SEAEDASLLSFMQGYMK | 2 | b5 | 1098.0 | 676.3 | 73 | 361552 | 56530 | 15.6 | | | | | | |
| Approt. E | P02649 | LGPLVEQGR | 113 | y3 | 554.8 | 360.2 | 46 | 233128 | 83398 | 35.8 | 1.45 | 0.14 | 9.5 | 1.30 | 0.11 | 8.9 |
| Approt. E | P02649 | LGPLVEQGR | 113 | b4 | 554.8 | 521.4 | 46 | 111405 | 35756 | 32.1 | 1.19 | 0.11 | 9.3 | | | |
| Approt. E | P02649 | LGPLVEQGR | 113 | y7 | 554.8 | 798.4 | 46 | 40683 | 15445 | 38.0 | 1.23 | 0.10 | 7.9 | | | |
| Approt. E | P02649 | LGPLVEQGR | 113 | y8 | 554.8 | 855.5 | 46 | 83226 | 31530 | 37.9 | 1.32 | 0.17 | 13.0 | | | |
| Approt. E | P02649 | LGPLVEQGR | 117 | y3 | 556.8 | 360.2 | 46 | 159398 | 54478 | 34.2 | | | | | | |
| Approt. E | P02649 | LGPLVEQGR | 117 | b4 | 556.8 | 525.4 | 46 | 93664 | 29455 | 31.4 | | | | | | |
| Approt. E | P02649 | LGPLVEQGR | 117 | y7 | 556.8 | 798.4 | 46 | 33217 | 12891 | 38.8 | | | | | | |
| Approt. E | P02649 | LGPLVEQGR | 117 | y8 | 556.8 | 855.5 | 46 | 65506 | 29657 | 45.3 | | | | | | |
| Approt. E | P02649 | LQAEAFQAR | 113 | y5 | 587.3 | 592.3 | 47 | 100039 | 29667 | 29.7 | 0.88 | 0.04 | 4.5 | 1.00 | 0.09 | 8.9 |
| Approt. E | P02649 | LQAEAFQAR | 113 | b3 | 587.3 | 453.3 | 47 | 81084 | 25420 | 31.4 | 1.03 | 0.06 | 5.5 | | | |
| Approt. E | P02649 | LQAEAFQAR | 113 | b4 | 587.3 | 582.3 | 47 | 85153 | 24683 | 29.0 | 1.09 | 0.08 | 7.5 | | | |
| Approt. E | P02649 | LQAEAFQAR | 113 | y7 | 587.3 | 792.4 | 47 | 60288 | 16736 | 27.8 | 1.00 | 0.06 | 6.3 | | | |
| Approt. E | P02649 | LQAEAFQAR | 117 | y5 | 589.3 | 592.3 | 47 | 114922 | 36418 | 31.7 | | | | | | |
| Approt. E | P02649 | LQAEAFQAR | 117 | b3 | 589.3 | 457.3 | 47 | 79029 | 24809 | 31.4 | | | | | | |
| Approt. E | P02649 | LQAEAFQAR | 117 | b4 | 589.3 | 586.3 | 47 | 78387 | 23218 | 29.6 | | | | | | |
| Approt. E | P02649 | LQAEAFQAR | 117 | y7 | 589.3 | 792.4 | 47 | 60731 | 18331 | 30.2 | | | | | | |
| Beta-2-gprot. I | P02749 | ATFGCHDGYSLDGPEEIECTK | 113 | y4 | 667.3 | 677.3 | 51 | 808149 | 123913 | 15.3 | 1.16 | 0.04 | 3.9 | 1.16 | 0.12 | 10.0 |
| Beta-2-gprot. I | P02749 | ATFGCHDGYSLDGPEEIECTK | 113 | y3 | 667.3 | 548.3 | 51 | 797369 | 121264 | 15.2 | 1.10 | 0.04 | 3.6 | | | |
| Beta-2-gprot. I | P02749 | ATFGCHDGYSLDGPEEIECTK | 113 | y4 | 889.4 | 677.3 | 62 | 263910 | 27935 | 10.6 | 1.33 | 0.10 | 7.8 | | | |
| Beta-2-gprot. I | P02749 | ATFGCHDGYSLDGPEEIECTK | 113 | b3 | 667.3 | 460.3 | 51 | 201820 | 32828 | 16.3 | 1.07 | 0.05 | 4.8 | | | |
| Beta-2-gprot. I | P02749 | ATFGCHDGYSLDGPEEIECTK | 117 | y4 | 669.3 | 681.3 | 51 | 699234 | 115567 | 16.5 | | | | | | |
| Beta-2-gprot. I | P02749 | ATFGCHDGYSLDGPEEIECTK | 117 | y3 | 669.3 | 552.3 | 51 | 728276 | 117836 | 16.2 | | | | | | |
| Beta-2-gprot. I | P02749 | ATFGCHDGYSLDGPEEIECTK | 117 | y4 | 892.1 | 681.3 | 63 | 200318 | 33122 | 16.5 | | | | | | |
| Beta-2-gprot. I | P02749 | ATFGCHDGYSLDGPEEIECTK | 117 | b3 | 669.3 | 464.3 | 51 | 189077 | 31788 | 16.8 | | | | | | |
| Beta-2-gprot. I | P02749 | ATVVYQGER | 113 | b3 | 581.8 | 412.3 | 47 | 792184 | 149442 | 18.9 | 1.03 | 0.04 | 3.7 | 1.00 | 0.03 | 3.3 |
| Beta-2-gprot. I | P02749 | ATVVYQGER | 113 | b4 | 581.8 | 511.3 | 47 | 308448 | 68740 | 22.3 | 1.01 | 0.05 | 5.0 | | | |
| Beta-2-gprot. I | P02749 | ATVVYQGER | 113 | y6 | 581.8 | 751.4 | 47 | 224637 | 52481 | 23.4 | 0.99 | 0.05 | 5.1 | | | |
| Beta-2-gprot. I | P02749 | ATVVYQGER | 113 | y7 | 581.8 | 850.4 | 47 | 82281 | 21633 | 26.3 | 0.95 | 0.06 | 5.9 | | | |
| Beta-2-gprot. I | P02749 | ATVVYQGER | 117 | b3 | 583.8 | 416.3 | 47 | 771609 | 155247 | 20.1 | | | | | | |
| Beta-2-gprot. I | P02749 | ATVVYQGER | 117 | b4 | 583.8 | 515.3 | 47 | 307379 | 70917 | 23.1 | | | | | | |
| Beta-2-gprot. I | P02749 | ATVVYQGER | 117 | y6 | 583.8 | 751.4 | 47 | 227513 | 59412 | 26.1 | | | | | | |
| Beta-2-gprot. I | P02749 | ATVVYQGER | 117 | y7 | 583.8 | 850.4 | 47 | 86884 | 24065 | 27.7 | | | | | | |
| Beta-2-gprot. I | P02749 | VCPFAGILENGAVR | 113 | y5 | 821.9 | 516.3 | 59 | 210437 | 65131 | 31.0 | 1.29 | 0.10 | 7.9 | 1.34 | 0.03 | 2.6 |
| Beta-2-gprot. I | P02749 | VCPFAGILENGAVR | 113 | y7 | 821.9 | 758.4 | 59 | 89714 | 26833 | 29.9 | 1.35 | 0.08 | 6.0 | | | |
| Beta-2-gprot. I | P02749 | VCPFAGILENGAVR | 113 | y9 | 821.9 | 928.5 | 59 | 90065 | 25819 | 28.7 | 1.37 | 0.14 | 10.5 | | | |
| Beta-2-gprot. I | P02749 | VCPFAGILENGAVR | 113 | y12 | 821.9 | 1243.7 | 59 | 127376 | 33760 | 26.5 | 1.35 | 0.13 | 9.8 | | | |
| Beta-2-gprot. I | P02749 | VCPFAGILENGAVR | 117 | y5 | 823.9 | 516.3 | 59 | 162700 | 49282 | 30.3 | | | | | | |
| Beta-2-gprot. I | P02749 | VCPFAGILENGAVR | 117 | y7 | 823.9 | 758.4 | 59 | 66053 | 18819 | 28.5 | | | | | | |
| Beta-2-gprot. I | P02749 | VCPFAGILENGAVR | 117 | y9 | 823.9 | 928.5 | 59 | 65133 | 16070 | 24.7 | | | | | | |
| Beta-2-gprot. I | P02749 | VCPFAGILENGAVR | 117 | y12 | 823.9 | 1243.7 | 59 | 93069 | 19029 | 20.4 | | | | | | |
| Pls. protease C1 IP | P05155 | FQPTLLTLPR | 113 | y8 | 663.4 | 910.6 | 51 | 319652 | 59322 | 18.6 | 0.88 | 0.04 | 4.6 | 0.99 | 0.08 | 8.0 |
| Pls. protease C1 IP | P05155 | FQPTLLTLPR | 113 | y4 | 663.4 | 486.3 | 51 | 235258 | 29815 | 12.7 | 1.00 | 0.09 | 8.8 | | | |
| Pls. protease C1 IP | P05155 | FQPTLLTLPR | 113 | y5 | 663.4 | 599.4 | 51 | 120619 | 17947 | 14.9 | 1.04 | 0.07 | 6.8 | | | |
| Pls. protease C1 IP | P05155 | FQPTLLTLPR | 113 | y9 | 663.4 | 1038.6 | 51 | 70061 | 13916 | 19.9 | 1.04 | 0.09 | 8.5 | | | |
| Pls. protease C1 IP | P05155 | FQPTLLTLPR | 117 | y8 | 665.4 | 910.6 | 51 | 364012 | 56733 | 15.6 | | | | | | |
| Pls. protease C1 IP | P05155 | FQPTLLTLPR | 117 | y4 | 665.4 | 486.3 | 51 | 235775 | 28711 | 12.2 | | | | | | |
| Pls. protease C1 IP | P05155 | FQPTLLTLPR | 117 | y5 | 665.4 | 599.4 | 51 | 116261 | 18714 | 16.1 | | | | | | |
| Pls. protease C1 IP | P05155 | FQPTLLTLPR | 117 | y9 | 665.4 | 1038.6 | 51 | 672831 | 2478 | | 18.5 | | | | | |
| Pls. protease C1 IP | P05155 | LEDMEQALSPSVFK | 113 | b3 | 625.3 | 498.3 | 49 | 738865 | 185938 | 25.2 | 0.94 | 0.08 | 8.3 | 1.00 | 0.06 | 5.6 |
| Pls. protease C1 IP | P05155 | LEDMEQALSPSVFK | 113 | b4 | 625.3 | 629.3 | 49 | 186047 | 45967 | 24.7 | 0.97 | 0.09 | 8.8 | | | |
| Pls. protease C1 IP | P05155 | LEDMEQALSPSVFK | 113 | b5 | 625.3 | 758.3 | 49 | 166539 | 44359 | 26.6 | 1.07 | 0.11 | 10.4 | | | |
| Pls. protease C1 IP | P05155 | LEDMEQALSPSVFK | 113 | y4 | 625.3 | 620.4 | 49 | 121940 | 31341 | 25.7 | 1.01 | 0.08 | 8.2 | | | |
| Pls. protease C1 IP | P05155 | LEDMEQALSPSVFK | 117 | b3 | 628.0 | 502.3 | 49 | 787923 | 214376 | 27.2 | | | | | | |
| Pls. protease C1 IP | P05155 | LEDMEQALSPSVFK | 117 | b4 | 628.0 | 633.3 | 49 | 193748 | 50488 | 26.1 | | | | | | |
| PIs. protease C1 IP | P05155 | LEDMEQALSPSVFK | 117 | b5 | 628.0 | 762.3 | 49 | 154373 | 31657 | 20.5 | | | | | | |
| Pls. protease C1 IP | P05155 | LEDMEQALSPSVFK | 117 | y4 | 628.0 | 624.4 | 49 | 119537 | 25180 | 21.1 | | | | | | |
| Pls. protease C1 IP | P05155 | LLDSLPSDTR | 113 | b3 | 628.8 | 482.3 | 49 | 334940 | 36686 | 11.0 | 1.24 | 0.10 | 7.8 | 1.39 | 0.12 | 8.8 |
| Pls. protease C1 IP | P05155 | LLDSLPSDTR | 113 | y7 | 628.8 | 775.4 | 49 | 199716 | 21156 | 10.6 | 1.46 | 0.13 | 9.2 | | | |
| Pls. protease C1 IP | P05155 | LLDSLPSDTR | 113 | y5 | 628.8 | 575.3 | 49 | 140783 | 17562 | 12.5 | 1.33 | 0.08 | 6.0 | | | |
| Pls. protease C1 IP | P05155 | LLDSLPSDTR | 113 | b4 | 628.8 | 569.3 | 49 | 120077 | 16915 | 14.1 | 1.51 | 0.11 | 7.5 | | | |
| Pls. protease C1 IP | P05155 | LLDSLPSDTR | 117 | b3 | 630.8 | 486.3 | 50 | 271247 | 35320 | 13.0 | | | | | | |
| Pls. protease C1 IP | P05155 | LLDSLPSDTR | 117 | y7 | 630.8 | 775.4 | 50 | 137642 | 20522 | 14.9 | | | | | | |
| Pls. protease C1 IP | P05155 | LLDSLPSDTR | 117 | y5 | 630.8 | 575.3 | 50 | 105883 | 14210 | 13.4 | | | | | | |
| Pls. protease C1 IP | P05155 | LLDSLPSDTR | 117 | b4 | 630.8 | 573.3 | 50 | 79837 | 10353 | 13.0 | | | | | | |
| Ceruloplasmin | P00453 | ALYLQYTDETFR | 113 | b3 | 554.0 | 488.3 | 46 | 460028 | 78255 | 17.0 | 1.05 | 0.06 | 5.7 | 0.96 | 0.06 | 6.6 |
| Ceruloplasmin | P00458 | ALYLQYTDETFR | 113 | b3 | 830.4 | 488.3 | 60 | 189991 | 53275 | 28.0 | 0.95 | 0.04 | 4.7 | | | |
| Ceruloplasmin | P00456 | ALYLQYTDETFR | 113 | y4 | 830.4 | 552.3 | 60 | 129024 | 39841 | 30.9 | 0.92 | 0.06 | 6.8 | | | |
| Ceruloplasmin | P00459 | ALYLQYTDETFR | 113 | y5 | 830.4 | 667.3 | 60 | 21157 | 6213 | 29.4 | 0.92 | 0.08 | 9.2 | | | |
| Ceruloplasmin | P00453 | ALYLQYTDETFR | 117 | b3 | 555.3 | 492.3 | 46 | 437917 | 74052 | 16.9 | | | | | | |
| Ceruloplasmin | P00458 | ALYLQYTDETFR | 117 | b3 | 832.4 | 492.3 | 60 | 198766 | 51976 | 26.1 | | | | | | |
| Ceruloplasmin | P00456 | ALYLQYTDETFR | 117 | y4 | 832.4 | 552.3 | 60 | 140859 | 42446 | 30.1 | | | | | | |
| Ceruloplasmin | P00459 | ALYLQYTDETFR | 117 | y5 | 832.4 | 667.3 | 60 | 23222 | 6888 | 29.7 | | | | | | |
| Ceruloplasmin | P00464 | DIASGLIGPLIICK | 113 | b5 | 584.0 | 584.3 | 47 | 1502573 | 254458 | 16.9 | 1.13 | 0.08 | 7.1 | 1.06 | 0.07 | 6.6 |
| Ceruloplasmin | P00469 | DIASGLIGPLIICK | 113 | b5 | 875.5 | 584.3 | 62 | 563561 | 161788 | 28.7 | 1.10 | 0.06 | 5.8 | | | |
| Ceruloplasmin | P00461 | DIASGLIGPLIICK | 113 | y4 | 584.0 | 673.4 | 47 | 253352 | 42669 | 16.8 | 0.99 | 0.09 | 9.5 | | | |
| Ceruloplasmin | P00463 | DIASGLIGPLIICK | 113 | b6 | 584.0 | 697.4 | 47 | 575416 | 78982 | 13.7 | 1.01 | 0.10 | 9.4 | | | |
| Ceruloplasmin | P00464 | DIASGLIGPLIICK | 117 | b5 | 586.7 | 588.3 | 47 | 1338288 | 265021 | 19.8 | | | | | | |
| Ceruloplasmin | P00469 | DIASGLIGPLIICK | 117 | b5 | 879.5 | 588.3 | 62 | 509785 | 128501 | 25.2 | | | | | | |
| Ceruloplasmin | P00461 | DIASGLIGPLIICK | 117 | y4 | 586.7 | 677.4 | 47 | 257995 | 42512 | 16.5 | | | | | | |
| Ceruloplasmin | P00463 | DIASGLIGPLIICK | 117 | b6 | 586.7 | 701.4 | 47 | 576700 | 113192 | 19.6 | | | | | | |
| Ceruloplasmin | P00477 | EVGPTNADPVCLAK | 113 | Y6 | 876.0 | 827.5 | 62 | 481880 | 96872 | 20.1 | 1.01 | 0.05 | 4.5 | 1.03 | 0.06 | 5.5 |
| Ceruloplasmin | P00476 | EVGPTNADPVCLAK | 113 | B8 | 876.0 | 924.4 | 62 | 91499 | 21773 | 23.8 | 0.97 | 0.09 | 9.2 | | | |
| Ceruloplasmin | P00473 | EVGPTNADPVCLAK | 113 | B6 | 876.0 | 738.4 | 62 | 122245 | 32126 | 26.3 | 1.05 | 0.11 | 10.6 | | | |
| Ceruloplasmin | P00472 | EVGPTNADPVCLAK | 113 | Y4 | 876.0 | 631.4 | 62 | 367683 | 86729 | 23.6 | 1.10 | 0.06 | 5.7 | | | |
| Ceruloplasmin | P00477 | EVGPTNADPVCLAK | 117 | Y6 | 880.0 | 831.5 | 62 | 480419 | 110970 | 23.1 | | | | | | |
| Ceruloplasmin | P00476 | EVGPTNADPVCLAK | 117 | B8 | 880.0 | 928.4 | 62 | 95064 | 22490 | 23.7 | | | | | | |
| Ceruloplasmin | P00473 | EVGPTNADPVCLAK | 117 | B6 | 880.0 | 742.4 | 62 | 115869 | 25391 | 21.9 | | | | | | |
| Ceruloplasmin | P00472 | EVGPTNADPVCLAK | 117 | Y4 | 880.0 | 635.4 | 62 | 334824 | 79906 | 23.9 | | | | | | |
| Ceruloplasmin | P00461 | GAYPLSIEPIGVR | 113 | b3 | 504.6 | 432.2 | 43 | 2630721 | 448091 | 17.0 | 0.87 | 0.02 | 2.5 | 0.95 | 0.06 | 6.1 |
| Ceruloplasmin | P00461 | GAYPLSIEPIGVR | 113 | b3 | 756.4 | 432.2 | 56 | 1463345 | 166572 | 11.4 | 1.01 | 0.06 | 6.0 | | | |
| Ceruloplasmin | P00461 | GAYPLSIEPIGVR | 113 | y5 | 756.4 | 541.3 | 56 | 1184065 | 167437 | 14.1 | 0.95 | 0.02 | 2.3 | | | |
| Ceruloplasmin | P00461 | GAYPLSIEPIGVR | 113 | y10 | 756.4 | 1080.6 | 56 | 803281 | 107439 | 13.4 | 0.99 | 0.04 | 3.7 | | | |
| Ceruloplasmin | P00461 | GAYPLSIEPIGVR | 117 | b3 | 506.0 | 436.2 | 43 | 3011442 | 528050 | 17.5 | | | | | | |
| Ceruloplasmin | P00461 | GAYPLSIEPIGVR | 117 | b3 | 758.4 | 436.2 | 56 | 1459730 | 201664 | 13.8 | | | | | | |
| Ceruloplasmin | P00461 | GAYPLSIEPIGVR | 117 | y5 | 758.4 | 541.3 | 56 | 1114861 | 426954 | 38.3 | | | | | | |
| Ceruloplasmin | P00461 | GAYPLSIEPIGVR | 117 | y10 | 758.4 | 1080.6 | 56 | 815454 | 106346 | 13.0 | | | | | | |
| Comp. factor B | P00751 | DISEVVTPR | 113 | y5 | 578.3 | 571.4 | 47 | 140798 | 38818 | 27.6 | 0.55 | 0.03 | 5.3 | 0.98 | 0.29 | 29.7 |
| Comp. factor B | P00751 | DISEVVTPR | 113 | b4 | 578.3 | 585.3 | 47 | 344028 | 99184 | 28.8 | 1.15 | 0.08 | 6.9 | | | |
| Comp. factor B | P00751 | DISEVVTPR | 113 | b3 | 578.3 | 456.3 | 47 | 154813 | 43976 | 28.4 | 1.08 | 0.07 | 6.1 | | | |
| Comp. factor B | P00751 | DISEVVTPR | 113 | b5 | 578.3 | 684.4 | 47 | 110830 | 33281 | 30.0 | 1.14 | 0.07 | 6.0 | | | |
| Comp. factor B | P00751 | DISEVVTPR | 117 | y5 | 580.3 | 571.4 | 47 | 260689 | 76302 | 29.3 | | | | | | |
| Comp. factor B | P00751 | DISEVVTPR | 117 | b4 | 580.3 | 589.3 | 47 | 297760 | 86355 | 29.0 | | | | | | |
| Comp. factor B | P00751 | DISEVVTPR | 117 | b3 | 580.3 | 460.3 | 47 | 142569 | 38280 | 26.9 | | | | | | |
| Comp. factor B | P00751 | DISEVVTPR | 117 | b5 | 580.3 | 688.4 | 47 | 98533 | 31770 | 32.2 | | | | | | |
| Comp. factor B | P00751 | EAGIPEFYDYDVALIK | 113 | b4 | 708.4 | 511.3 | 53 | 796519 | 205878 | 25.8 | 1.15 | 0.07 | 6.4 | 1.17 | 0.08 | 6.7 |
| Comp. factor B | P00751 | EAGIPEFYDYDVALIK | 113 | y3 | 708.4 | 513.4 | 53 | 199355 | 52463 | 26.3 | 1.20 | 0.08 | 7.0 | | | |
| Comp. factor B | P00751 | EAGIPEFYDYDVALIK | 113 | y4 | 708.4 | 584.4 | 53 | 158089 | 45829 | 29.0 | 1.26 | 0.11 | 8.6 | | | |
| Comp. factor B | P00751 | EAGIPEFYDYDVALIK | 113 | b6 | 708.4 | 737.4 | 53 | 140713 | 37846 | 26.9 | 1.08 | 0.04 | 4.0 | | | |
| Comp. factor B | P00751 | EAGIPEFYDYDVALIK | 117 | b4 | 711.0 | 515.3 | 54 | 695704 | 195262 | 28.1 | | | | | | |
| Comp. factor B | P00751 | EAGIPEFYDYDVALIK | 117 | y3 | 711.0 | 517.4 | 54 | 166236 | 46777 | 28.1 | | | | | | |
| Comp. factor B | P00751 | EAGIPEFYDYDVALIK | 117 | y4 | 711.0 | 588.4 | 54 | 127478 | 43919 | 34.5 | | | | | | |
| Comp. factor B | P00751 | EAGIPEFYDYDVALIK | 117 | b6 | 711.0 | 741.4 | 54 | 130748 | 34303 | 26.2 | | | | | | |
| Comp. factor B | P00751 | EELLPAQDIK | 113 | b3 | 718.4 | 512.3 | 54 | 512035 | 80476 | 15.7 | 1.01 | 0.09 | 9.3 | 0.96 | 0.04 | 4.3 |
| Comp. factor B | P00751 | EELLPAQDIK | 113 | y6 | 718.4 | 811.5 | 54 | 139906 | 22155 | 15.8 | 0.93 | 0.10 | 11.2 | | | |
| Comp. factor B | P00751 | EELLPAQDIK | 113 | y3 | 718.4 | 515.3 | 54 | 109322 | 14441 | 13.2 | 0.99 | 0.13 | 13.3 | | | |
| Comp. factor B | P00751 | EELLPAQDIK | 113 | b4 | 718.4 | 625.4 | 54 | 76809 | 10382 | 13.5 | 0.92 | 0.10 | 10.5 | | | |
| Comp. factor B | P00751 | EELLPAQDIK | 117 | b3 | 722.4 | 516.3 | 54 | 507947 | 73767 | 14.5 | | | | | | |
| Comp. factor B | P00751 | EELLPAQDIK | 117 | y6 | 722.4 | 815.5 | 54 | 150624 | 24800 | 16.5 | | | | | | |
| Comp. factor B | P00751 | EELLPAQDIK | 117 | y3 | 722.4 | 519.3 | 54 | 112023 | 19476 | 17.4 | | | | | | |
| Comp. factor B | P00751 | EELLPAQDIK | 117 | b4 | 722.4 | 629.4 | 54 | 84121 | 15175 | 18.0 | | | | | | |
| Comp. factor B | P00751 | GDSGGPLIVHK | 113 | b5 | 453.9 | 514.2 | 41 | 543652 | 88552 | 16.3 | 0.92 | 0.03 | 3.4 | 0.92 | 0.04 | 4.4 |
| Comp. factor B | P00751 | GDSGGPLIVHK | 113 | b4 | 453.9 | 457.2 | 41 | 271318 | 51025 | 18.8 | 0.95 | 0.07 | 7.2 | | | |
| Comp. factor B | P00751 | GDSGGPLIVHK | 113 | b6 | 453.9 | 611.3 | 41 | 34825 | 5536 | 15.9 | 0.87 | 0.08 | 9.3 | | | |
| Comp. factor B | P00751 | GDSGGPLIVHK | 113 | y3 | 453.9 | 523.3 | 41 | 22463 | 3225 | 14.4 | 0.95 | 0.12 | 12.8 | | | |
| Comp. factor B | P00751 | GDSGGPLIVHK | 117 | b5 | 456.6 | 518.2 | 41 | 588925 | 95816 | 16.3 | | | | | | |
| Comp. factor B | P00751 | GDSGGPLIVHK | 117 | b4 | 456.6 | 461.2 | 41 | 286446 | 55166 | 19.3 | | | | | | |
| Comp. factor B | P00751 | GDSGGPLIVHK | 117 | b6 | 456.6 | 615.3 | 41 | 40580 | 7275 | 17.9 | | | | | | |
| Comp. factor B | P00751 | GDSGGPLIVHK | 117 | y3 | 456.6 | 527.3 | 41 | 24035 | 4374 | 18.2 | | | | | | |
| Comp. factor B | P00751 | VSEADSSNADWVTK | 113 | b3 | 597.0 | 456.3 | 48 | 636834 | 86740 | 13.6 | 1.09 | 0.04 | 3.4 | 1.07 | 0.02 | 2.2 |
| Comp. factor B | P00751 | VSEADSSNADWVTK | 113 | b5 | 597.0 | 642.3 | 48 | 434223 | 52047 | 12.0 | 1.07 | 0.04 | 4.0 | | | |
| Comp. factor B | P00751 | VSEADSSNADWVTK | 113 | b4 | 597.0 | 527.3 | 48 | 358036 | 48543 | 13.6 | 1.04 | 0.04 | 4.0 | | | |
| Comp. factor B | P00751 | VSEADSSNADWVTK | 113 | b6 | 597.0 | 729.3 | 48 | 68608 | 11180 | 16.3 | 1.08 | 0.07 | 6.9 | | | |
| Comp. factor B | P00751 | VSEADSSNADWVTK | 117 | b3 | 599.6 | 460.3 | 48 | 583146 | 84595 | 14.5 | | | | | | |
| Comp. factor B | P00751 | VSEADSSNADWVTK | 117 | b5 | 599.6 | 646.3 | 48 | 407909 | 54521 | 13.4 | | | | | | |
| Comp. factor B | P00751 | VSEADSSNADWVTK | 117 | b4 | 599.6 | 531.3 | 48 | 345716 | 47929 | 13.9 | | | | | | |
| Comp. factor B | P00751 | VSEADSSNADWVTK | 117 | b6 | 599.6 | 733.3 | 48 | 63692 | 9161 | 14.4 | | | | | | |
| Comp. factor B | P00751 | YGLVTYATYPK | 113 | b3 | 519.3 | 474.3 | 44 | 803168 | 199809 | 24.9 | 0.90 | 0.02 | 2.5 | 0.92 | 0.09 | 9.5 |
| Comp. factor B | P00751 | YGLVTYATYPK | 113 | b3 | 778.4 | 474.3 | 57 | 497600 | 142743 | 28.7 | 1.03 | 0.02 | 2.2 | | | |
| Comp. factor B | P00751 | YGLVTYATYPK | 113 | b4 | 519.3 | 573.3 | 44 | 144983 | 32182 | 22.2 | 0.94 | 0.03 | 2.8 | | | |
| Comp. factor B | P00751 | YGLVTYATYPK | 113 | b5 | 778.4 | 674.4 | 57 | 50408 | 15275 | 30.3 | 0.82 | 0.09 | 10.5 | | | |
| Comp. factor B | P00751 | YGLVTYATYPK | 117 | b3 | 522.0 | 478.3 | 44 | 889680 | 219769 | 24.7 | | | | | | |
| Comp. factor B | P00751 | YGLVTYATYPK | 117 | b3 | 782.4 | 478.3 | 57 | 482170 | 135196 | 28.0 | | | | | | |
| Comp. factor B | P00751 | YGLVTYATYPK | 117 | b4 | 522.0 | 577.3 | 44 | 154221 | 35172 | 22.8 | | | | | | |
| Comp. factor B | P00751 | YGLVTYATYPK | 117 | b5 | 782.4 | 678.4 | 57 | 61340 | 16635 | 27.1 | | | | | | |
| Comp. factor H | P08603 | DGWSAQPTCIK | 113 | y5 | 771.9 | 758.4 | 57 | 157818 | 61859 | 39.2 | 0.92 | 0.33 | 35.6 | 0.91 | 0.02 | 1.9 |
| Comp. factor H | P08603 | DGWSAQPTCIK | 113 | b3 | 771.9 | 499.2 | 57 | 112561 | 42216 | 37.5 | 0.93 | 0.35 | 37.0 | | | |
| Comp. factor H | P08603 | DGWSAQPTCIK | 113 | y3 | 771.9 | 560.3 | 57 | 77477 | 29048 | 37.5 | 0.91 | 0.33 | 36.2 | | | |
| Comp. factor H | P08603 | DGWSAQPTCIK | 113 | b4 | 771.9 | 586.3 | 57 | 65535 | 21765 | 33.2 | 0.89 | 0.32 | 35.8 | | | |
| Comp. factor H | P08603 | DGWSAQPTCIK | 117 | y5 | 775.9 | 762.4 | 57 | 170996 | 26506 | 15.5 | | | | | | |
| Comp. factor H | P08603 | DGWSAQPTCIK | 117 | b3 | 775.9 | 503.2 | 57 | 121160 | 20809 | 17.2 | | | | | | |
| Comp. factor H | P08603 | DGWSAQPTCIK | 117 | y3 | 775.9 | 564.3 | 57 | 85075 | 13384 | 15.7 | | | | | | |
| Comp. factor H | P08603 | DGWSAQPTCIK | 117 | b4 | 775.9 | 590.3 | 57 | 72383 | 10781 | 14.9 | | | | | | |
| Comp. factor H | P08603 | ECDTDGWTNDIPICEVVK | 113 | b3 | 608.5 | 545.2 | 48 | 205440 | 49811 | 24.2 | 1.11 | 0.06 | 5.4 | 1.08 | 0.03 | 3.2 |
| Comp. factor H | P08603 | ECDTDGWTNDIPICEVVK | 113 | b3 | 811.1 | 545.2 | 59 | 201890 | 72625 | 36.0 | 1.05 | 0.06 | 5.8 | | | |
| Comp. factor H | P08603 | ECDTDGWTNDIPICEVVK | 113 | b5 | 811.1 | 761.3 | 59 | 189467 | 63037 | 33.3 | 1.11 | 0.06 | 5.0 | | | |
| Comp. factor H | P08603 | ECDTDGWTNDIPICEVVK | 113 | y5 | 811.1 | 774.4 | 59 | 120855 | 38962 | 32.2 | 1.06 | 0.03 | 2.4 | | | |
| Comp. factor H | P08603 | ECDTDGWTNDIPICEVVK | 117 | b3 | 610.5 | 549.2 | 49 | 184901 | 42491 | 23.0 | | | | | | |
| Comp. factor H | P08603 | ECDTDGWTNDIPICEVVK | 117 | b3 | 813.7 | 549.2 | 59 | 191002 | 65190 | 34.1 | | | | | | |
| Comp. factor H | P08603 | ECDTDGWTNDIPICEVVK | 117 | b5 | 813.7 | 765.3 | 59 | 170938 | 59763 | 35.0 | | | | | | |
| Comp. factor H | P08603 | ECDTDGWTNDIPICEVVK | 117 | y5 | 813.7 | 778.4 | 59 | 114157 | 35875 | 31.4 | | | | | | |
| Comp. factor H | P08603 | LSYTCEGGFR | 113 | y4 | 665.3 | 436.2 | 51 | 489902 | 123118 | 25.1 | 1.11 | 0.05 | 4.1 | 1.07 | 0.05 | 4.8 |
| Comp. factor H | P08603 | LSYTCEGGFR | 113 | y5 | 665.3 | 565.3 | 51 | 84054 | 21186 | 25.2 | 1.00 | 0.09 | 9.2 | | | |
| Comp. factor H | P08603 | LSYTCEGGFR | 113 | y9 | 665.3 | 1076.4 | 51 | 125337 | 33886 | 27.0 | 1.10 | 0.07 | 6.6 | | | |
| Comp. factor H | P08603 | LSYTCEGGFR | 113 | y8 | 665.3 | 989.4 | 51 | 43022 | 10994 | 25.6 | 1.07 | 0.10 | 9.2 | | | |
| Comp. factor H | P08603 | LSYTCEGGFR | 117 | y4 | 667.3 | 436.2 | 51 | 440280 | 108124 | 24.6 | | | | | | |
| Comp. factor H | P08603 | LSYTCEGGFR | 117 | y5 | 667.3 | 565.3 | 51 | 85757 | 24146 | 28.2 | | | | | | |
| Comp. factor H | P08603 | LSYTCEGGFR | 117 | y9 | 667.3 | 1076.4 | 51 | 113528 | 29491 | 26.0 | | | | | | |
| Comp. factor H | P08603 | LSYTCEGGFR | 117 | y8 | 667.3 | 989.4 | 51 | 40651 | 11062 | 27.2 | | | | | | |
| Comp. factor H | P08603 | SSNLIILEEHLK | 113 | b3 | 559.3 | 429.2 | 46 | 1598632 | 296412 | 18.5 | 1.07 | 0.05 | 4.9 | 1.10 | 0.02 | 2.1 |
| Comp. factor H | P08603 | SSNLIILEEHLK | 113 | b4 | 559.3 | 542.3 | 46 | 982198 | 184210 | 18.8 | 1.12 | 0.04 | 3.1 | | | |
| Comp. factor H | P08603 | SSNLIILEEHLK | 113 | b5 | 559.3 | 655.4 | 46 | 221893 | 46161 | 20.8 | 1.11 | 0.05 | 4.2 | | | |
| Comp. factor H | P08603 | SSNLIILEEHLK | 113 | y3 | 559.3 | 537.4 | 46 | 120551 | 22428 | 18.6 | 1.09 | 0.06 | 5.3 | | | |
| Comp. factor H | P08603 | SSNLIILEEHLK | 117 | b3 | 562.0 | 433.2 | 46 | 1487181 | 246468 | 16.6 | | | | | | |
| Comp. factor H | P08603 | SSNLIILEEHLK | 117 | b4 | 562.0 | 546.3 | 46 | 873839 | 168061 | 19.2 | | | | | | |
| Comp. factor H | P08603 | SSNLIILEEHLK | 117 | b5 | 562.0 | 659.4 | 46 | 200156 | 38022 | 19.0 | | | | | | |
| Comp. factor H | P08603 | SSNLIILEEHLK | 117 | y3 | 562.0 | 541.4 | 46 | 111769 | 24349 | 21.8 | | | | | | |
| Comp. factor H | P08603 | WQSIPLCVEK | 113 | y6 | 770.4 | 885.5 | 57 | 195440 | 28366 | 14.5 | 0.98 | 0.05 | 4.9 | 1.00 | 0.06 | 5.9 |
| Comp. factor H | P08603 | WQSIPLCVEK | 113 | y4 | 770.4 | 675.3 | 57 | 217748 | 36068 | 16.6 | 0.95 | 0.03 | 3.3 | | | |
| Comp. factor H | P08603 | WQSIPLCVEK | 113 | y3 | 770.4 | 515.3 | 57 | 166396 | 24193 | 14.5 | 0.99 | 0.03 | 2.5 | | | |
| Comp. factor H | P08603 | WQSIPLCVEK | 113 | b3 | 770.4 | 542.3 | 57 | 182485 | 26221 | 14.4 | 1.09 | 0.06 | 5.4 | | | |
| Comp. factor H | P08603 | WQSIPLCVEK | 117 | y6 | 774.4 | 889.5 | 57 | 200732 | 32108 | 16.0 | | | | | | |
| Comp. factor H | P08603 | WQSIPLCVEK | 117 | y4 | 774.4 | 679.4 | 57 | 228078 | 34447 | 15.1 | | | | | | |
| Comp. factor H | P08603 | WQSIPLCVEK | 117 | y3 | 774.4 | 519.3 | 57 | 167923 | 25742 | 15.3 | | | | | | |
| Comp. factor H | P08603 | WQSIPLCVEK | 117 | b3 | 774.4 | 546.3 | 57 | 168173 | 26496 | 15.8 | | | | | | |
| Clusterin | P10909 | ASSIIDELFQDR | 113 | y4 | 511.9 | 565.3 | 44 | 167487 | 19345 | 11.5 | 0.85 | 0.06 | 7.3 | 1.10 | 0.17 | 15.2 |
| Clusterin | P10909 | ASSIIDELFQDR | 113 | b3 | 767.4 | 386.2 | 56 | 1166459 | 188738 | 16.2 | 1.17 | 0.08 | 6.7 | | | |
| Clusterin | P10909 | ASSIIDELFQDR | 113 | y6 | 767.4 | 807.4 | 56 | 596283 | 90258 | 15.1 | 1.19 | 0.10 | 8.3 | | | |
| Clusterin | P10909 | ASSIIDELFQDR | 113 | y5 | 767.4 | 678.4 | 56 | 422924 | 65785 | 15.6 | 1.19 | 0.07 | 6.3 | | | |
| Clusterin | P10909 | ASSIIDELFQDR | 117 | y4 | 513.3 | 565.3 | 44 | 197046 | 15586 | 7.9 | | | | | | |
| Clusterin | P10909 | ASSIIDELFQDR | 117 | b3 | 769.4 | 390.2 | 56 | 998721 | 151169 | 15.1 | | | | | | |
| Clusterin | P10909 | ASSIIDELFQDR | 117 | y6 | 769.4 | 807.4 | 56 | 503442 | 82179 | 16.3 | | | | | | |
| Clusterin | P10909 | ASSIIDELFQDR | 117 | y5 | 769.4 | 678.4 | 56 | 356841 | 56195 | 15.7 | | | | | | |
| Clusterin | P10909 | ELDESLQVAER | 113 | b3 | 714.9 | 498.3 | 54 | 588752 | 103419 | 17.6 | 1.15 | 0.04 | 3.9 | 1.16 | 0.02 | 1.5 |
| Clusterin | P10909 | ELDESLQVAER | 113 | y7 | 714.9 | 802.4 | 54 | 200890 | 38983 | 19.4 | 1.16 | 0.09 | 8.0 | | | |
| Clusterin | P10909 | ELDESLQVAER | 113 | y8 | 714.9 | 931.5 | 54 | 187932 | 30565 | 16.3 | 1.15 | 0.08 | 7.4 | | | |
| Clusterin | P10909 | ELDESLQVAER | 113 | y9 | 714.9 | 1046.5 | 54 | 80996 | 11970 | 14.8 | 1.18 | 0.09 | 7.9 | | | |
| Clusterin | P10909 | ELDESLQVAER | 117 | b3 | 716.9 | 502.3 | 54 | 512228 | 79980 | 15.6 | | | | | | |
| Clusterin | P10909 | ELDESLQVAER | 117 | y7 | 716.9 | 802.4 | 54 | 171316 | 23041 | 13.4 | | | | | | |
| Clusterin | P10909 | ELDESLQVAER | 117 | y8 | 716.9 | 931.5 | 54 | 163566 | 24556 | 15.0 | | | | | | |
| Clusterin | P10909 | ELDESLQVAER | 117 | y9 | 716.9 | 1046.5 | 54 | 68458 | 9366 | 13.7 | | | | | | |
| Clusterin | P10909 | LFDSDPITVTVPVEVSR | 113 | b5 | 672.1 | 718.3 | 52 | 1729858 | 180097 | 10.4 | 1.29 | 0.05 | 3.8 | 1.20 | 0.12 | 10.2 |
| Clusterin | P10909 | LFDSDPITVTVPVEVSR | 113 | y6 | 672.1 | 686.4 | 52 | 988182 | 99520 | 10.1 | 1.04 | 0.05 | 4.6 | | | |
| Clusterin | P10909 | LFDSDPITVTVPVEVSR | 113 | b6 | 672.1 | 815.4 | 52 | 102553 | 16200 | 15.8 | 1.29 | 0.12 | 9.5 | | | |
| Clusterin | P10909 | LFDSDPITVTVPVEVSR | 113 | y7 | 672.1 | 785.5 | 52 | 132359 | 11946 | 9.0 | 1.18 | 0.09 | 7.8 | | | |
| Clusterin | P10909 | LFDSDPITVTVPVEVSR | 117 | b5 | 673.4 | 722.3 | 52 | 1339976 | 112434 | 8.4 | | | | | | |
| Clusterin | P10909 | LFDSDPITVTVPVEVSR | 117 | y6 | 673.4 | 686.4 | 52 | 957140 | 112882 | 11.8 | | | | | | |
| Clusterin | P10909 | LFDSDPITVTVPVEVSR | 117 | b6 | 673.4 | 819.4 | 52 | 79586 | 12937 | 16.3 | | | | | | |
| Clusterin | P10909 | LFDSDPITVTVPVEVSR | 117 | y7 | 673.4 | 785.5 | 52 | 112819 | 12755 | 11.3 | | | | | | |
| Comp. C3 | P01024 | EYVLPSFEVIVEPTEK | 113 | b3 | 720.4 | 532.3 | 54 | 1591257 | 254520 | 16.0 | 0.96 | 0.05 | 4.9 | 0.97 | 0.06 | 5.9 |
| Comp. C3 | P01024 | EYVLPSFEVIVEPTEK | 113 | b4 | 720.4 | 645.4 | 54 | 963242 | 117358 | 12.2 | 0.89 | 0.04 | 4.3 | | | |
| Comp. C3 | P01024 | EYVLPSFEVIVEPTEK | 113 | y3 | 720.4 | 517.3 | 54 | 395521 | 48409 | 12.2 | 0.99 | 0.06 | 6.3 | | | |
| Comp. C3 | P01024 | EYVLPSFEVIVEPTEK | 113 | b2 | 720.4 | 433.2 | 54 | 1290916 | 205894 | 15.9 | 1.02 | 0.04 | 3.5 | | | |
| Comp. C3 | P01024 | EYVLPSFEVIVEPTEK | 117 | b3 | 723.1 | 536.3 | 54 | 1652163 | 246375 | 14.9 | | | | | | |
| Comp. C3 | P01024 | EYVLPSFEVIVEPTEK | 117 | b4 | 723.1 | 649.4 | 54 | 1086157 | 127228 | 11.7 | | | | | | |
| Comp. C3 | P01024 | EYVLPSFEVIVEPTEK | 117 | y3 | 723.1 | 521.3 | 54 | 400440 | 56123 | 14.0 | | | | | | |
| Comp. C3 | P01024 | EYVLPSFEVIVEPTEK | 117 | b2 | 723.1 | 437.2 | 54 | 1269505 | 219591 | 17.3 | | | | | | |
| Comp. C3 | P01024 | ILLQGTPVAQMTEDAVDAER | 113 | b3 | 766.4 | 480.4 | 56 | 1337943 | 323037 | 24.1 | 1.30 | 0.05 | 3.8 | 1.31 | 0.04 | 3.4 |
| Comp. C3 | P01024 | ILLQGTPVAQMTEDAVDAER | 113 | b4 | 766.4 | 608.4 | 56 | 515926 | 130789 | 25.4 | 1.33 | 0.09 | 6.4 | | | |
| Comp. C3 | P01024 | ILLQGTPVAQMTEDAVDAER | 113 | b5 | 766.4 | 665.4 | 56 | 406918 | 104418 | 25.7 | 1.25 | 0.09 | 7.3 | | | |
| Comp. C3 | P01024 | ILLQGTPVAQMTEDAVDAER | 113 | b6 | 766.4 | 766.5 | 56 | 318528 | 82330 | 25.8 | 1.36 | 0.07 | 5.1 | | | |
| Comp. C3 | P01024 | ILLQGTPVAQMTEDAVDAER | 117 | b3 | 767.7 | 484.4 | 56 | 1037225 | 273444 | 26.4 | | | | | | |
| Comp. C3 | P01024 | ILLQGTPVAQMTEDAVDAER | 117 | b4 | 767.7 | 612.4 | 56 | 388908 | 99716 | 25.6 | | | | | | |
| Comp. C3 | P01024 | ILLQGTPVAQMTEDAVDAER | 117 | b5 | 767.7 | 669.4 | 56 | 324000 | 75374 | 23.3 | | | | | | |
| Comp. C3 | P01024 | ILLQGTPVAQMTEDAVDAER | 117 | b6 | 767.7 | 770.5 | 56 | 234098 | 53961 | 23.1 | | | | | | |
| Comp. C3 | P01024 | IPIEDGSGEVVLSR | 113 | b3 | 537.6 | 464.3 | 45 | 230510 | 52384 | 22.7 | 0.85 | 0.04 | 4.5 | 1.05 | 0.14 | 13.2 |
| Comp. C3 | P01024 | IPIEDGSGEVVLSR | 113 | y5 | 805.9 | 573.4 | 58 | 146105 | 39596 | 27.1 | 1.11 | 0.07 | 6.0 | | | |
| Comp. C3 | P01024 | IPIEDGSGEVVLSR | 113 | y9 | 805.9 | 903.5 | 58 | 416895 | 90040 | 21.6 | 1.13 | 0.07 | 6.2 | | | |
| Comp. C3 | P01024 | IPIEDGSGEVVLSR | 113 | y13 | 805.9 | 1357.7 | 58 | 210445 | 30947 | 14.7 | 1.13 | 0.08 | 7.0 | | | |
| Comp. C3 | P01024 | IPIEDGSGEVVLSR | 117 | b3 | 539.0 | 468.3 | 45 | 273181 | 63120 | 23.1 | | | | | | |
| Comp. C3 | P01024 | IPIEDGSGEVVLSR | 117 | y5 | 807.9 | 573.4 | 58 | 131647 | 33820 | 25.7 | | | | | | |
| Comp. C3 | P01024 | IPIEDGSGEVVLSR | 117 | y9 | 807.9 | 903.5 | 58 | 368590 | 78756 | 21.4 | | | | | | |
| Comp. C3 | P01024 | IPIEDGSGEVVLSR | 117 | y13 | 807.9 | 1357.7 | 58 | 186463 | 23655 | 12.7 | | | | | | |
| Comp. C3 | P01024 | SSLSVPYVIVPLK | 113 | y3 | 561.3 | 497.3 | 46 | 6888113 | 2809928 | 40.8 | 1.14 | 0.07 | 6.6 | 1.16 | 0.03 | 2.6 |
| Comp. C3 | P01024 | SSLSVPYVIVPLK | 113 | b3 | 561.3 | 428.3 | 46 | 2196343 | 869164 | 39.6 | 1.17 | 0.06 | 5.0 | | | |
| Comp. C3 | P01024 | SSLSVPYVIVPLK | 113 | y4 | 561.3 | 596.4 | 46 | 2046293 | 772046 | 37.7 | 1.19 | 0.08 | 6.5 | | | |
| Comp. C3 | P01024 | SSLSVPYVIVPLK | 113 | b4 | 561.3 | 515.3 | 46 | 2122612 | 818759 | 38.6 | 1.12 | 0.06 | 5.2 | | | |
| Comp. C3 | P01024 | SSLSVPYVIVPLK | 117 | y3 | 564.0 | 501.4 | 46 | 5965097 | 2188899 | 36.7 | | | | | | |
| Comp. C3 | P01024 | SSLSVPYVIVPLK | 117 | b3 | 564.0 | 432.3 | 46 | 1869305 | 733246 | 39.2 | | | | | | |
| Comp. C3 | P01024 | SSLSVPYVIVPLK | 117 | y4 | 564.0 | 600.4 | 46 | 1736216 | 672386 | 38.7 | | | | | | |
| Comp. C3 | P01024 | SSLSVPYVIVPLK | 117 | b4 | 564.0 | 519.3 | 46 | 1894733 | 741552 | 39.1 | | | | | | |
| Comp. C3 | P01024 | TGLQEVEVK | 113 | b4 | 641.9 | 540.3 | 50 | 741323 | 61060 | 8.2 | 1.14 | 0.04 | 3.8 | 1.19 | 0.05 | 3.8 |
| Comp. C3 | P01024 | TGLQEVEVK | 113 | b5 | 641.9 | 669.4 | 50 | 621754 | 66486 | 10.7 | 1.21 | 0.06 | 5.2 | | | |
| Comp. C3 | P01024 | TGLQEVEVK | 113 | y4 | 641.9 | 614.4 | 50 | 395126 | 55024 | 13.9 | 1.24 | 0.06 | 4.8 | | | |
| Comp. C3 | P01024 | TGLQEVEVK | 113 | b6 | 641.9 | 768.4 | 50 | 159149 | 21362 | 13.4 | 1.17 | 0.10 | 8.5 | | | |
| Comp. C3 | P01024 | TGLQEVEVK | 117 | b4 | 645.9 | 544.3 | 50 | 648571 | 39928 | 6.2 | | | | | | |
| Comp. C3 | P01024 | TGLQEVEVK | 117 | b5 | 645.9 | 673.4 | 50 | 514077 | 55888 | 10.9 | | | | | | |
| Comp. C3 | P01024 | TGLQEVEVK | 117 | y4 | 645.9 | 618.4 | 50 | 318522 | 45266 | 14.2 | | | | | | |
| Comp. C3 | P01024 | TGLQEVEVK | 117 | b6 | 645.9 | 772.4 | 50 | 137186 | 19956 | 14.5 | | | | | | |
| Comp.C4-A a chain | P01028 α | GLEEELQFSLGSK | 113 | b3 | 573.0 | 440.3 | 47 | 435871 | 91148 | 20.9 | 1.04 | 0.08 | 7.4 | 1.01 | 0.04 | 3.5 |
| Comp.C4-A a chain | P01028 α | GLEEELQFSLGSK | 113 | y3 | 573.0 | 431.3 | 47 | 394130 | 84374 | 21.4 | 0.96 | 0.08 | 8.4 | | | |
| Comp.C4-A a chain | P01028 α | GLEEELQFSLGSK | 113 | b4 | 573.0 | 569.3 | 47 | 286480 | 54407 | 19.0 | 1.04 | 0.07 | 6.9 | | | |
| Comp.C4-A a chain | P01028 α | GLEEELQFSLGSK | 113 | b5 | 573.0 | 698.3 | 47 | 281342 | 68087 | 24.2 | 1.00 | 0.09 | 8.8 | | | |
| Comp.C4-A a chain | P01028 α | GLEEELQFSLGSK | 117 | b3 | 575.6 | 444.3 | 47 | 423413 | 102303 | 24.2 | | | | | | |
| Comp.C4-A a chain | P01028 α | GLEEELQFSLGSK | 117 | y3 | 575.6 | 435.3 | 47 | 412525 | 98204 | 23.8 | | | | | | |
| Comp.C4-A a chain | P01028 α | GLEEELQFSLGSK | 117 | b4 | 575.6 | 573.3 | 47 | 279327 | 64279 | 23.0 | | | | | | |
| Comp.C4-A a chain | P01028 α | GLEEELQFSLGSK | 117 | b5 | 575.6 | 702.3 | 47 | 281341 | 67985 | 24.2 | | | | | | |
| Comp.C4-A a chain | P01028 α | LGQYASPTAK | 113 | b3 | 658.4 | 439.3 | 51 | 676908 | 225911 | 33.4 | 1.31 | 0.09 | 7.2 | 1.29 | 0.08 | 6.3 |
| Comp.C4-A a chain | P01028 α | LGQYASPTAK | 113 | y4 | 658.4 | 556.4 | 51 | 528675 | 188563 | 35.7 | 1.34 | 0.09 | 6.8 | | | |
| Comp.C4-A a chain | P01028 α | LGQYASPTAK | 113 | y3 | 658.4 | 459.3 | 51 | 231591 | 73894 | 31.9 | 1.17 | 0.09 | 8.1 | | | |
| Comp.C4-A a chain | P01028 α | LGQYASPTAK | 113 | b4 | 658.4 | 602.3 | 51 | 178737 | 63043 | 35.3 | 1.34 | 0.08 | 5.7 | | | |
| Comp.C4-A a chain | P01028 α | LGQYASPTAK | 117 | b3 | 662.4 | 443.3 | 51 | 522804 | 185736 | 35.5 | | | | | | |
| Comp.C4-A a chain | P01028 α | LGQYASPTAK | 117 | y4 | 662.4 | 560.4 | 51 | 396565 | 140206 | 35.4 | | | | | | |
| Comp.C4-A a chain | P01028 α | LGQYASPTAK | 117 | y3 | 662.4 | 463.3 | 51 | 201114 | 68483 | 34.1 | | | | | | |
| Comp.C4-A a chain | P01028 α | LGQYASPTAK | 117 | b4 | 662.4 | 606.3 | 51 | 134106 | 50430 | 37.6 | | | | | | |
| Comp.C4-A a chain | P01028 α | VLSLAQEQVGGSPEK | 113 | y3 | 608.0 | 513.3 | 48 | 782074 | 100034 | 12.8 | 1.80 | 0.08 | 4.4 | 1.80 | 0.08 | 4.7 |
| Comp.C4-A a chain | P01028 α | VLSLAQEQVGGSPEK | 113 | y6 | 608.0 | 714.4 | 48 | 285007 | 36219 | 12.7 | 1.86 | 0.09 | 4.7 | | | |
| Comp.C4-A a chain | P01028 α | VLSLAQEQVGGSPEK | 113 | b5 | 608.0 | 624.4 | 48 | 506224 | 66937 | 13.2 | 1.85 | 0.08 | 4.4 | | | |
| Comp.C4-A a chain | P01028 α | VLSLAQEQVGGSPEK | 113 | y4 | 608.0 | 600.3 | 48 | 99653 | 14847 | 14.9 | 1.68 | 0.17 | 10.0 | | | |
| Comp.C4-A a chain | P01028 α | VLSLAQEQVGGSPEK | 117 | y3 | 610.7 | 517.3 | 49 | 433887 | 53039 | 12.2 | | | | | | |
| Comp.C4-A a chain | P01028 α | VLSLAQEQVGGSPEK | 117 | y6 | 610.7 | 718.4 | 49 | 153262 | 19135 | 12.5 | | | | | | |
| Comp.C4-A a chain | P01028 α | VLSLAQEQVGGSPEK | 117 | b5 | 610.7 | 628.4 | 49 | 273894 | 36716 | 13.4 | | | | | | |
| Comp.C4-A a chain | P01028 α | VLSLAQEQVGGSPEK | 117 | y4 | 610.7 | 604.3 | 49 | 59752 | 9141 | 15.3 | | | | | | |
| Comp.C4-A b chain | P01028 α | AEFQDALEK | 113 | b5 | 665.9 | 731.3 | 51 | 235172 | 75678 | 32.2 | 0.88 | 0.03 | 3.5 | 0.96 | 0.08 | 8.2 |
| Comp.C4-A b chain | P01028 α | AEFQDALEK | 113 | y3 | 665.9 | 529.3 | 51 | 180067 | 56890 | 31.6 | 0.98 | 0.06 | 6.4 | | | |
| Comp.C4-A b chain | P01028 α | AEFQDALEK | 113 | y5 | 665.9 | 715.4 | 51 | 75977 | 24864 | 32.7 | 0.93 | 0.10 | 11.1 | | | |
| Comp.C4-A b chain | P01028 α | AEFQDALEK | 113 | b4 | 665.9 | 616.3 | 51 | 98080 | 32238 | 32.9 | 1.06 | 0.12 | 10.9 | | | |
| Comp.C4-A b chain | P01028 α | AEFQDALEK | 117 | b5 | 669.9 | 735.3 | 51 | 268571 | 87683 | 32.6 | | | | | | |
| Comp.C4-A b chain | P01028 α | AEFQDALEK | 117 | y3 | 669.9 | 533.3 | 51 | 186689 | 65461 | 35.1 | | | | | | |
| Comp.C4-A b chain | P01028 α | AEFQDALEK | 117 | y5 | 669.9 | 719.4 | 51 | 83697 | 31107 | 37.2 | | | | | | |
| Comp.C4-A b chain | P01028 α | AEFQDALEK | 117 | b4 | 669.9 | 620.3 | 51 | 91576 | 27328 | 29.8 | | | | | | |
| Comp.C4-A b chain | P01028 α | LNMGITDLQGLR | 113 | b4 | 735.9 | 556.3 | 55 | 122894 | 35318 | 28.7 | 1.08 | 0.11 | 10.4 | 1.12 | 0.08 | 7.0 |
| Comp.C4-A b chain | P01028 α | LNMGITDLQGLR | 113 | b3 | 735.9 | 499.3 | 55 | 66010 | 15529 | 23.5 | 1.24 | 0.15 | 12.5 | | | |
| Comp.C4-A b chain | P01028 α | LNMGITDLQGLR | 113 | y9 | 735.9 | 972.5 | 55 | 40737 | 12296 | 30.2 | 1.10 | 0.14 | 12.9 | | | |
| Comp.C4-A b chain | P01028 α | LNMGITDLQGLR | 113 | b7 | 735.9 | 885.5 | 55 | 27329 | 7766 | 28.4 | 1.07 | 0.19 | 17.4 | | | |
| Comp.C4-A b chain | P01028 α | LNMGITDLQGLR | 117 | b4 | 737.9 | 560.3 | 55 | 112086 | 21421 | 19.1 | | | | | | |
| Comp.C4-A b chain | P01028 α | LNMGITDLQGLR | 117 | b3 | 737.9 | 503.3 | 55 | 54020 | 14249 | 26.4 | | | | | | |
| Comp.C4-A b chain | P01028 α | LNMGITDLQGLR | 117 | y9 | 737.9 | 972.5 | 55 | 37842 | 13672 | 36.1 | | | | | | |
| Comp.C4-A b chain | P01028 α | LNMGITDLQGLR | 117 | b7 | 737.9 | 889.5 | 55 | 25644 | 5835 | 22.8 | | | | | | |
| Comp.C4-A b chain | P01028 α | VDFTLSSER | 113 | y7 | 597.3 | 839.4 | 48 | 137128 | 19056 | 13.9 | 0.86 | 0.05 | 6.2 | 0.98 | 0.11 | 11.3 |
| Comp.C4-A b chain | P01028 α | VDFTLSSER | 113 | b3 | 597.3 | 502.3 | 48 | 174065 | 22072 | 12.7 | 1.12 | 0.09 | 8.4 | | | |
| Comp.C4-A b chain | P01028 α | VDFTLSSER | 113 | y6 | 597.3 | 692.4 | 48 | 82300 | 11023 | 13.4 | 0.94 | 0.06 | 6.0 | | | |
| Comp C4-A b chain | P01028 α | VDFTLSSER | 113 | y8 | 597.3 | 954.5 | 48 | 60221 | 9231 | 15.3 | 1.02 | 0.07 | 7.3 | | | |
| Comp.C4-A b chain | P01028 α | VDFTLSSER | 117 | y7 | 599.3 | 839.4 | 48 | 159667 | 21001 | 13.2 | | | | | | |
| Comp.C4-A b chain | P01028 α | VDFTLSSER | 117 | b3 | 599.3 | 506.3 | 48 | 156148 | 23534 | 15.1 | | | | | | |
| Comp.C4-A b chain | P01028 α | VDFTLSSER | 117 | y6 | 599.3 | 692.4 | 48 | 87617 | 13032 | 14.9 | | | | | | |
| Comp.C4-A b chain | P01028 α | VDFTLSSER | 117 | y8 | 599.3 | 954.5 | 48 | 59141 | 7317 | 12.4 | | | | | | |
| Comp.C4-A b chain | P01028 α | VGDTLNLNLR | 113 | y7 | 627.9 | 843.5 | 49 | 225411 | 36291 | 16.1 | 1.26 | 0.10 | 7.9 | 1.27 | 0.02 | 1.4 |
| Comp.C4-A b chain | P01028 α | VGDTLNLNLR | 113 | y6 | 627.9 | 742.5 | 49 | 94642 | 12888 | 13.6 | 1.29 | 0.12 | 9.6 | | | |
| Comp.C4-A b chain | P01028 α | VGDTLNLNLR | 113 | y9 | 627.9 | 1015.6 | 49 | 113565 | 21488 | 18.9 | 1.26 | 0.12 | 9.4 | | | |
| Comp.C4-A b chain | P01028 α | VGDTLNLNLR | 113 | b4 | 627.9 | 513.3 | 49 | 260442 | 34550 | 13.3 | 1.29 | 0.15 | 11.8 | | | |
| Comp.C4-A b chain | P01028 α | VGDTLNLNLR | 117 | y7 | 629.9 | 843.5 | 49 | 179169 | 26408 | 14.7 | | | | | | |
| Comp.C4-A b chain | P01028 α | VGDTLNLNLR | 117 | y6 | 629.9 | 742.5 | 49 | 73600 | 8342 | 11.3 | | | | | | |
| Comp.C4-A b chain | P01028 α | VGDTLNLNLR | 117 | y9 | 629.9 | 1015.6 | 49 | 91011 | 19594 | 21.5 | | | | | | |
| Comp.C4-A b chain | P01028 α | VGDTLNLNLR | 117 | b4 | 629.9 | 517.3 | 49 | 201641 | 16680 | 8.3 | | | | | | |
| Comp.cmp C9 | P02748 | AIEDYINEFSVR | 113 | b4 | 798.4 | 569.3 | 58 | 64123 | 16806 | 26.2 | 1.11 | 0.18 | 15.9 | 1.09 | 0.12 | 10.9 |
| Comp.cmp C9 | P02748 | AIEDYINEFSVR | 113 | y4 | 798.4 | 508.3 | 58 | 37736 | 9466 | 25.1 | 1.07 | 0.09 | 8.1 | | | |
| Comp.cmp C9 | P02748 | AIEDYINEFSVR | 113 | b5 | 798.4 | 732.4 | 58 | 22760 | 5353 | 23.5 | 0.94 | 0.09 | 10.0 | | | |
| Comp.cmp C9 | P02748 | AIEDYINEFSVR | 113 | y7 | 798.4 | 864.5 | 58 | 7803 | 2698 | 34.6 | 1.23 | 0.25 | 20.1 | | | |
| Comp.cmp C9 | P02748 | AIEDYINEFSVR | 117 | b4 | 800.4 | 573.3 | 58 | 59157 | 18113 | 30.6 | | | | | | |
| Comp.cmp C9 | P02748 | AIEDYINEFSVR | 117 | y4 | 800.4 | 508.3 | 58 | 35524 | 8701 | 24.5 | | | | | | |
| Comp.cmp C9 | P02748 | AIEDYINEFSVR | 117 | b5 | 800.4 | 736.4 | 58 | 24366 | 6293 | 25.8 | | | | | | |
| Comp.cmp C9 | P02748 | AIEDYINEFSVR | 117 | y7 | 800.4 | 864.5 | 58 | 6407 | 1918 | 29.9 | | | | | | |
| Comp.cmp C9 | P02748 | LSPIYNLVPVK | 113 | y4 | 762.0 | 483.3 | 56 | 401579 | 151594 | 37.7 | 1.05 | 0.09 | 8.7 | 1.16 | 0.18 | 15.5 |
| Comp.cmp C9 | P02748 | LSPIYNLVPVK | 113 | y4 | 508.3 | 483.3 | 43 | 184592 | 23250 | 12.6 | 1.41 | 0.13 | 8.9 | | | |
| Comp.cmp C9 | P02748 | LSPIYNLVPVK | 113 | b6 | 762.0 | 828.5 | 56 | 48687 | 16490 | 33.9 | 1.01 | 0.06 | 6.3 | | | |
| Comp.cmp C9 | P02748 | LSPIYNLVPVK | 113 | b5 | 762.0 | 714.4 | 56 | 43326 | 17406 | 40.2 | 1.16 | 0.22 | 18.8 | | | |
| Comp.cmp C9 | P02748 | LSPIYNLVPVK | 117 | y4 | 766.0 | 487.3 | 56 | 380590 | 139967 | 36.8 | | | | | | |
| Comp.cmp C9 | P02748 | LSPIYNLVPVK | 117 | y4 | 511.0 | 487.3 | 44 | 132260 | 22224 | 16.8 | | | | | | |
| Comp.cmp C9 | P02748 | LSPIYNLVPVK | 117 | b6 | 766.0 | 832.5 | 56 | 48479 | 16834 | 34.7 | | | | | | |
| Comp.cmp C9 | P02748 | LSPIYNLVPVK | 117 | b5 | 766.0 | 718.4 | 56 | 36912 | 13812 | 37.4 | | | | | | |
| Comp.cmp C9 | P02748 | VVEESELAR | 113 | y5 | 586.3 | 575.3 | 47 | 55008 | 7361 | 13.4 | 0.92 | 0.06 | 7.0 | 1.00 | 0.06 | 6.0 |
| Comp.cmp C9 | P02748 | VVEESELAR | 113 | y3 | 586.3 | 359.2 | 47 | 80904 | 7717 | 9.5 | 1.03 | 0.07 | 6.9 | | | |
| Comp.cmp C9 | P02748 | VVEESELAR | 113 | y8 | 586.3 | 932.5 | 47 | 32530 | 6488 | 19.9 | 1.00 | 0.09 | 9.0 | | | |
| Comp.cmp C9 | P02748 | VVEESELAR | 113 | b4 | 586.3 | 597.3 | 47 | 36352 | 3966 | 10.9 | 1.07 | 0.11 | 9.8 | | | |
| Comp.cmp C9 | P02748 | VVEESELAR | 117 | y5 | 588.3 | 575.3 | 47 | 59634 | 7116 | 11.9 | | | | | | |
| Comp.cmp C9 | P02748 | VVEESELAR | 117 | y3 | 588.3 | 359.2 | 47 | 79153 | 8995 | 11.4 | | | | | | |
| Comp.cmp C9 | P02748 | VVEESELAR | 117 | y8 | 588.3 | 932.5 | 47 | 32325 | 5223 | 16.2 | | | | | | |
| Comp.cmp C9 | P02748 | VVEESELAR | 117 | b4 | 588.3 | 601.3 | 47 | 34235 | 3872 | 11.3 | | | | | | |
| Alpha-2-HS-gprot. | P02765 | EHAVEGDCDFQLLK | 113 | y3 | 647.7 | 513.4 | 50 | 695450 | 135632 | 19.5 | 1.25 | 0.05 | 4.4 | 1.23 | 0.04 | 3.2 |
| Alpha-2-HS-gprot. | P02765 | EHAVEGDCDFQLLK | 113 | b3 | 486.0 | 478.2 | 42 | 1241688 | 135748 | 10.9 | 1.17 | 0.06 | 5.3 | | | |
| Alpha-2-HS-gprot. | P02765 | EHAVEGDCDFQLLK | 113 | b5 | 647.7 | 706.4 | 50 | 188882 | 37473 | 19.8 | 1.25 | 0.08 | 6.6 | | | |
| Alpha-2-HS-gprot. | P02765 | EHAVEGDCDFQLLK | 113 | y3 | 486.0 | 513.4 | 42 | 357184 | 45333 | 12.7 | 1.25 | 0.06 | 4.8 | | | |
| Alpha-2-HS-gprot. | P02765 | EHAVEGDCDFQLLK | 117 | y3 | 650.3 | 517.4 | 51 | 555528 | 104221 | 18.8 | | | | | | |
| Alpha-2-HS-gprot. | P02765 | EHAVEGDCDFQLLK | 117 | b3 | 488.0 | 482.2 | 42 | 1062309 | 110650 | 10.4 | | | | | | |
| Alpha-2-HS-gprot. | P02765 | EHAVEGDCDFQLLK | 117 | b5 | 650.3 | 710.4 | 51 | 150614 | 25151 | 16.7 | | | | | | |
| Alpha-2-HS-gprot. | P02765 | EHAVEGDCDFQLLK | 117 | y3 | 488.0 | 517.4 | 42 | 287277 | 38766 | 13.5 | | | | | | |
| Alpha-2-HS-gprot. | P02765 | FSVVYAK | 113 | y3 | 547.3 | 521.3 | 45 | 992526 | 365400 | 36.8 | 1.25 | 0.04 | 2.8 | 1.36 | 0.08 | 6.0 |
| Alpha-2-HS-gprot. | P02765 | FSVVYAK | 113 | b3 | 547.3 | 474.3 | 45 | 748065 | 275882 | 36.9 | 1.36 | 0.03 | 2.1 | | | |
| Alpha-2-HS-gprot. | P02765 | FSVVYAK | 113 | y4 | 547.3 | 620.4 | 45 | 314976 | 121242 | 38.5 | 1.39 | 0.03 | 2.5 | | | |
| Alpha-2-HS-gprot. | P02765 | FSVVYAK | 113 | b4 | 547.3 | 573.3 | 45 | 139923 | 55304 | 39.5 | 1.45 | 0.09 | 6.3 | | | |
| Alpha-2-HS-gprot. | P02765 | FSVVYAK | 117 | y3 | 551.3 | 525.3 | 46 | 790072 | 289446 | 36.6 | | | | | | |
| Alpha-2-HS-gprot. | P02765 | FSVVYAK | 117 | b3 | 551.3 | 478.3 | 46 | 553900 | 204540 | 36.9 | | | | | | |
| Alpha-2-HS-gprot. | P02765 | FSVVYAK | 117 | y4 | 551.3 | 624.4 | 46 | 225695 | 84496 | 37.4 | | | | | | |
| Alpha-2-HS-gprot. | P02765 | FSVVYAK | 117 | b4 | 551.3 | 577.3 | 46 | 95528 | 35170 | 36.8 | | | | | | |
| Alpha-2-HS-gprot. | P02765 | HTLNQIDEVK | 113 | y4 | 492.9 | 630.4 | 43 | 100317 | 29402 | 29.3 | 1.07 | 0.08 | 8.0 | 1.05 | 0.03 | 2.5 |
| Alpha-2-HS-gprot. | P02765 | HTLNQIDEVK | 113 | y3 | 492.9 | 515.3 | 43 | 139593 | 29255 | 21.0 | 1.06 | 0.07 | 6.5 | | | |
| Alpha-2-HS-gprot. | P02765 | HTLNQIDEVK | 113 | b4 | 492.9 | 606.3 | 43 | 132705 | 33562 | 25.3 | 1.01 | 0.06 | 5.5 | | | |
| Alpha-2-HS-gprot. | P02765 | HTLNQIDEVK | 113 | b5 | 492.9 | 734.4 | 43 | 24775 | 7418 | 29.9 | 1.07 | 0.14 | 13.2 | | | |
| Alpha-2-HS-gprot. | P02765 | HTLNQIDEVK | 117 | y4 | 495.6 | 634.4 | 43 | 93607 | 23356 | 25.0 | | | | | | |
| Alpha-2-HS-gprot. | P02765 | HTLNQIDEVK | 117 | y3 | 495.6 | 519.3 | 43 | 132552 | 33402 | 25.2 | | | | | | |
| Alpha-2-HS-gprot. | P02765 | HTLNQIDEVK | 117 | b4 | 495.6 | 610.3 | 43 | 131072 | 32886 | 25.1 | | | | | | |
| Alpha-2-HS-gprot. | P02765 | HTLNQIDEVK | 117 | b5 | 495.6 | 738.4 | 43 | 22928 5004 | 21.8 | | | | | | | |
| Fibronectin | P02751 | DLQFVEVTDVK | 113 | b3 | 525.0 | 497.3 | 44 | 326342 | 59803 | 18.3 | 1.31 | 0.04 | 3.1 | 1.09 | 0.15 | 13.4 |
| Fibronectin | P02751 | DLQFVEVTDVK | 113 | b3 | 786.9 | 497.3 | 57 | 126435 | 31202 | 24.7 | 1.06 | 0.07 | 6.6 | | | |
| Fibronectin | P02751 | DLQFVEVTDVK | 113 | y3 | 786.9 | 501.3 | 57 | 18492 | 6276 | 33.9 | 1.02 | 0.19 | 18.9 | | | |
| Fibronectin | P02751 | DLQFVEVTDVK | 113 | b4 | 786.9 | 644.3 | 57 | 36414 | 10081 | 27.7 | 0.99 | 0.13 | 13.4 | | | |
| Fibronectin | P02751 | DLQFVEVTDVK | 117 | b3 | 527.6 | 501.3 | 44 | 250505 | 50100 | 20.0 | | | | | | |
| Fibronectin | P02751 | DLQFVEVTDVK | 117 | b3 | 790.9 | 501.3 | 58 | 119806 | 28953 | 24.2 | | | | | | |
| Fibronectin | P02751 | DLQFVEVTDVK | 117 | y3 | 790.9 | 505.3 | 58 | 17901 | 4310 | 24.1 | | | | | | |
| Fibronectin | P02751 | DLQFVEVTDVK | 117 | b4 | 790.9 | 648.3 | 58 | 36941 | 8551 | 23.1 | | | | | | |
| Fibronectin | P02751 | IYLYTLNDNAR | 113 | b3 | 748.4 | 530.3 | 55 | 129447 | 18933 | 14.6 | 1.60 | 0.19 | 12.0 | 1.52 | 0.15 | 9.6 |
| Fibronectin | P02751 | IYLYTLNDNAR | 113 | y5 | 748.4 | 589.3 | 55 | 43698 | 7899 | 18.1 | 1.34 | 0.17 | 12.9 | | | |
| Fibronectin | P02751 | IYLYTLNDNAR | 113 | y4 | 748.4 | 475.2 | 55 | 63953 | 6980 | 10.9 | 1.48 | 0.12 | 8.1 | | | |
| Fibronectin | P02751 | IYLYTLNDNAR | 113 | y8 | 748.4 | 966.5 | 55 | 77455 | 9057 | 11.7 | 1.67 | 0.16 | 9.5 | | | |
| Fibronectin | P02751 | IYLYTLNDNAR | 117 | b3 | 750.4 | 534.3 | 56 | 81365 | 11566 | 14.2 | | | | | | |
| Fibronectin | P02751 | IYLYTLNDNAR | 117 | y5 | 750.4 | 589.3 | 56 | 32590 | 3809 | 11.7 | | | | | | |
| Fibronectin | P02751 | IYLYTLNDNAR | 117 | y4 | 750.4 | 475.2 | 56 | 43274 | 5320 | 12.3 | | | | | | |
| Fibronectin | P02751 | IYLYTLNDNAR | 117 | y8 | 750.4 | 966.5 | 56 | 46383 | 4880 | 10.5 | | | | | | |
| Fibronectin | P02751 | STTPDITGYR | 113 | y5 | 625.8 | 609.3 | 49 | 320151 | 29267 | 9.1 | 0.95 | 0.04 | 3.9 | 0.99 | 0.09 | 9.0 |
| Fibronectin | P02751 | STTPDITGYR | 113 | y7 | 625.8 | 821.4 | 49 | 105977 | 17831 | 16.8 | 1.08 | 0.07 | 6.7 | | | |
| Fibronectin | P02751 | STTPDITGYR | 113 | b3 | 625.8 | 430.2 | 49 | 76307 | 8805 | 11.5 | 0.89 | 0.08 | 8.8 | | | |
| Fibronectin | P02751 | STTPDITGYR | 113 | b5 | 625.8 | 642.3 | 49 | 84403 | 7718 | 9.1 | 1.05 | 0.09 | 8.2 | | | |
| Fibronectin | P02751 | STTPDITGYR | 117 | y5 | 627.8 | 609.3 | 49 | 336240 | 31047 | 9.2 | | | | | | |
| Fibronectin | P02751 | STTPDITGYR | 117 | y7 | 627.8 | 821.4 | 49 | 98684 | 19034 | 19.3 | | | | | | |
| Fibronectin | P02751 | STTPDITGYR | 117 | b3 | 627.8 | 434.2 | 49 | 86129 | 6465 | 7.5 | | | | | | |
| Fibronectin | P02751 | STTPDITGYR | 117 | b5 | 627.8 | 646.3 | 49 | 80569 | 8259 | 10.3 | | | | | | |
| Fibronectin | P02751 | WLPSSSPVTGYR | 113 | y6 | 745.4 | 692.4 | 55 | 126520 | 42508 | 33.6 | 0.75 | 0.04 | 5.2 | 1.04 | 0.20 | 18.7 |
| Fibronectin | P02751 | WLPSSSPVTGYR | 113 | y10 | 745.4 | 1050.5 | 55 | 369920 | 98375 | 26.6 | 1.14 | 0.02 | 2.1 | | | |
| Fibronectin | P02751 | WLPSSSPVTGYR | 113 | y7 | 745.4 | 779.4 | 55 | 65988 | 21225 | 32.2 | 1.14 | 0.14 | 12.2 | | | |
| Fibronectin | P02751 | WLPSSSPVTGYR | 113 | y11 | 745.4 | 1163.6 | 55 | 77117 | 21777 | 28.2 | 1.14 | 0.08 | 7.2 | | | |
| Fibronectin | P02751 | WLPSSSPVTGYR | 117 | y6 | 747.4 | 692.4 | 55 | 166879 | 51129 | 30.6 | | | | | | |
| Fibronectin | P02751 | WLPSSSPVTGYR | 117 | y10 | 747.4 | 1050.5 | 55 | 324965 | 87220 | 26.8 | | | | | | |
| Fibronectin | P02751 | WLPSSSPVTGYR | 117 | y7 | 747.4 | 779.4 | 55 | 59090 | 19622 | 33.2 | | | | | | |
| Fibronectin | P02751 | WLPSSSPVTGYR | 117 | y11 | 747.4 | 1163.6 | 55 | 66955 | 17095 | 25.5 | | | | | | |
| Gelsolin, isoform 1 | P06396 | AGALNSNDAFVLK | 113 | y3 | 534.0 | 499.4 | 45 | 84890 | 31346 | 36.9 | 0.94 | 0.34 | 35.5 | 1.00 | 0.08 | 7.8 |
| Gelsolin, isoform 1 | P06396 | AGALNSNDAFVLK | 113 | b5 | 534.0 | 567.3 | 45 | 123649 | 10320 | 8.3 | 1.11 | 0.05 | 4.9 | | | |
| Gelsolin, isoform 1 | P06396 | AGALNSNDAFVLK | 113 | b6 | 534.0 | 654.4 | 45 | 23461 | 2583 | 11.0 | 0.97 | 0.10 | 10.4 | | | |
| Gelsolin, isoform 1 | P06396 | AGALNSNDAFVLK | 113 | b8 | 534.0 | 883.4 | 45 | 16381 | 2522 | 15.4 | 0.96 | 0.12 | 12.1 | | | |
| Gelsolin, isoform 1 | P06396 | AGALNSNDAFVLK | 117 | y3 | 536.6 | 503.4 | 45 | 90082 | 7642 | 8.5 | | | | | | |
| Gelsolin, isoform 1 | P06396 | AGALNSNDAFVLK | 117 | b5 | 536.6 | 571.3 | 45 | 111563 | 11776 | 10.6 | | | | | | |
| Gelsolin, isoform 1 | P06396 | AGALNSNDAFVLK | 117 | b6 | 536.6 | 658.4 | 45 | 24345 | 3109 | 12.8 | | | | | | |
| Gelsolin, isoform 1 | P06396 | AGALNSNDAFVLK | 117 | b8 | 536.6 | 887.4 | 45 | 17161 | 1735 | 10.1 | | | | | | |
| Gelsolin, isoform 1 | P06396 | AQPVQVAEGSEPDGFWEALGGK | 113 | y3 | 638.8 | 401.3 | 50 | 984858 | 153922 | 15.6 | 1.18 | 0.04 | 3.4 | 1.19 | 0.05 | 4.6 |
| Gelsolin, isoform 1 | P06396 | AQPVQVAEGSEPDGFWEALGGK | 113 | b4 | 638.8 | 536.3 | 50 | 406468 | 73876 | 18.2 | 1.21 | 0.04 | 3.3 | | | |
| Gelsolin, isoform 1 | P06396 | AQPVQVAEGSEPDGFWEALGGK | 113 | b5 | 638.8 | 664.4 | 50 | 354763 | 57424 | 16.2 | 1.12 | 0.06 | 5.1 | | | |
| Gelsolin, isoform 1 | P06396 | AQPVQVAEGSEPDGFWEALGGK | 113 | y5 | 638.8 | 585.4 | 50 | 155974 | 27138 | 17.4 | 1.25 | 0.08 | 6.4 | | | |
| Gelsolin, isoform 1 | P06396 | AQPVQVAEGSEPDGFWEALGGK | 117 | y3 | 640.8 | 405.3 | 50 | 834779 | 149579 | 17.9 | | | | | | |
| Gelsolin, isoform 1 | P06396 | AQPVQVAEGSEPDGFWEALGGK | 117 | b4 | 640.8 | 540.3 | 50 | 335381 | 57259 | 17.1 | | | | | | |
| Gelsolin, isoform 1 | P06396 | AQPVQVAEGSEPDGFWEALGGK | 117 | b5 | 640.8 | 668.4 | 50 | 316264 | 49252 | 15.6 | | | | | | |
| Gelsolin, isoform 1 | P06396 | AQPVQVAEGSEPDGFWEALGGK | 117 | y5 | 640.8 | 589.4 | 50 | 124333 | 18964 | 15.3 | | | | | | |
| Gelsolin, isoform 1 | P06396 | TASDFITK | 113 | b4 | 581.8 | 515.3 | 47 | 275255 | 14697 | 5.3 | 1.01 | 0.05 | 4.5 | 0.99 | 0.04 | 4.1 |
| Gelsolin, isoform 1 | P06396 | TASDFITK | 113 | y3 | 581.8 | 501.3 | 47 | 71890 | 7731 | 10.8 | 1.02 | 0.08 | 7.8 | | | |
| Gelsolin, isoform 1 | P06396 | TASDFITK | 113 | y4 | 581.8 | 648.4 | 47 | 44453 | 5644 | 12.7 | 1.01 | 0.12 | 12.2 | | | |
| Gelsolin, isoform 1 | P06396 | TASDFITK | 113 | b5 | 581.8 | 662.3 | 47 | 35530 | 4433 | 12.5 | 0.93 | 0.08 | 8.2 | | | |
| Gelsolin, isoform 1 | P06396 | TASDFITK | 117 | b4 | 585.8 | 519.3 | 47 | 271313 | 9687 | 3.6 | | | | | | |
| Gelsolin, isoform 1 | P06396 | TASDFITK | 117 | y3 | 585.8 | 505.3 | 47 | 71056 | 8392 | 11.8 | | | | | | |
| Gelsolin, isoform 1 | P06396 | TASDFITK | 117 | y4 | 585.8 | 652.4 | 47 | 44612 | 8006 | 17.9 | | | | | | |
| Gelsolin, isoform 1 | P06396 | TASDFITK | 117 | b5 | 585.8 | 666.3 | 47 | 38225 | 4764 | 12.5 | | | | | | |
| Hemopexin | P02790 | DYFMPCPGR | 113 | b3 | 641.8 | 566.3 | 50 | 1115133 | 150315 | 13.5 | 0.85 | 0.05 | 5.3 | 0.89 | 0.04 | 4.6 |
| Hemopexin | P02790 | DYFMPCPGR | 113 | y6 | 641.8 | 717.3 | 50 | 541937 | 93891 | 17.3 | 0.92 | 0.03 | 3.3 | | | |
| Hemopexin | P02790 | DYFMPCPGR | 113 | y8 | 641.8 | 1027.4 | 50 | 319302 | 56603 | 17.7 | 0.92 | 0.04 | 3.8 | | | |
| Hemopexin | P02790 | DYFMPCPGR | 113 | b4 | 641.8 | 697.3 | 50 | 264672 | 42994 | 16.2 | 0.85 | 0.06 | 6.7 | | | |
| Hemopexin | P02790 | DYFMPCPGR | 117 | b3 | 643.8 | 570.3 | 50 | 1312404 | 190467 | 14.5 | | | | | | |
| Hemopexin | P02790 | DYFMPCPGR | 117 | y6 | 643.8 | 717.3 | 50 | 587352 | 102961 | 17.5 | | | | | | |
| Hemopexin | P02790 | DYFMPCPGR | 117 | y8 | 643.8 | 1027.4 | 50 | 348659 | 63020 | 18.1 | | | | | | |
| Hemopexin | P02790 | DYFMPCPGR | 117 | b4 | 643.8 | 701.3 | 50 | 312954 | 52068 | 16.6 | | | | | | |
| Hemopexin | P02790 | GECQAEGVLFFQGDR | 113 | b4 | 926.9 | 615.3 | 64 | 2634433 | 6345 | 13.8 | 0.98 | 0.06 | 6.1 | 0.99 | 0.04 | 4.4 |
| Hemopexin | P02790 | GECQAEGVLFFQGDR | 113 | b5 | 618.3 | 686.3 | 49 | 219291 | 28079 | 12.8 | 0.93 | 0.04 | 4.8 | | | |
| Hemopexin | P02790 | GECQAEGVLFFQGDR | 113 | b7 | 618.3 | 872.4 | 49 | 123392 | 17468 | 14.2 | 1.02 | 0.06 | 5.6 | | | |
| Hemopexin | P02790 | GECQAEGVLFFQGDR | 113 | b7 | 926.9 | 872.4 | 64 | 160187 | 14548 | 9.1 | 1.03 | 0.06 | 5.5 | | | |
| Hemopexin | P02790 | GECQAEGVLFFQGDR | 117 | b4 | 928.9 | 619.3 | 64 | 268600 | 30067 | 11.2 | | | | | | |
| Hemopexin | P02790 | GECQAEGVLFFQGDR | 117 | b5 | 619.6 | 690.3 | 49 | 236088 | 37218 | 15.8 | | | | | | |
| Hemopexin | P02790 | GECQAEGVLFFQGDR | 117 | b7 | 619.6 | 876.4 | 49 | 121060 | 17503 | 14.5 | | | | | | |
| Hemopexin | P02790 | GECQAEGVLFFQGDR | 117 | b7 | 928.9 | 876.4 | 64 | 156386 | 17286 | 11.1 | | | | | | |
| Hemopexin | P02790 | GGYTLVSGYPK | 113 | b4 | 474.6 | 519.3 | 42 | 191477 | 60618 | 31.7 | 0.84 | 0.08 | 10.1 | 0.96 | 0.08 | 8.1 |
| Hemopexin | P02790 | GGYTLVSGYPK | 113 | b4 | 711.4 | 519.3 | 54 | 681483 | 243251 | 35.7 | 0.99 | 0.05 | 5.4 | | | |
| Hemopexin | P02790 | GGYTLVSGYPK | 113 | y4 | 711.4 | 604.3 | 54 | 280075 | 100678 | 35.9 | 1.01 | 0.05 | 5.2 | | | |
| Hemopexin | P02790 | GGYTLVSGYPK | 113 | b5 | 711.4 | 632.3 | 54 | 456363 | 169063 | 37.0 | 0.99 | 0.03 | 3.1 | | | |
| Hemopexin | P02790 | GGYTLVSGYPK | 117 | b4 | 477.3 | 523.3 | 42 | 222368 | 64072 | 28.8 | | | | | | |
| Hemopexin | P02790 | GGYTLVSGYPK | 117 | b4 | 715.4 | 523.3 | 54 | 682967 | 250858 | 36.7 | | | | | | |
| Hemopexin | P02790 | GGYTLVSGYPK | 117 | y4 | 715.4 | 608.3 | 54 | 280071 | 104040 | 37.1 | | | | | | |
| Hemopexin | P02790 | GGYTLVSGYPK | 117 | b5 | 715.4 | 636.3 | 54 | 460295 | 163371 | 35.5 | | | | | | |
| Hemopexin | P02790 | LLQDEFPGIPSPLDAAVECHR | 113 | b4 | 835.5 | 739.4 | 60 | 1949769 | 487505 | 25.0 | 1.21 | 0.07 | 5.9 | 1.13 | 0.13 | 11.9 |
| Hemopexin | P02790 | LLQDEFPGIPSPLDAAVECHR | 113 | y8 | 835.5 | 957.4 | 60 | 359829 | 87060 | 24.2 | 0.93 | 0.05 | 5.0 | | | |
| Hemopexin | P02790 | LLQDEFPGIPSPLDAAVECHR | 113 | b5 | 835.5 | 886.5 | 60 | 478334 | 111024 | 23.2 | 1.20 | 0.06 | 5.3 | | | |
| Hemopexin | P02790 | LLQDEFPGIPSPLDAAVECHR | 113 | b3 | 835.5 | 610.4 | 60 | 2990960 | 792647 | 26.5 | 1.17 | 0.06 | 4.8 | | | |
| Hemopexin | P02790 | LLQDEFPGIPSPLDAAVECHR | 117 | b4 | 836.8 | 743.4 | 60 | 1621699 | 434524 | 26.8 | | | | | | |
| Hemopexin | P02790 | LLQDEFPGIPSPLDAAVECHR | 117 | y8 | 836.8 | 957.4 | 60 | 386914 | 82158 | 21.2 | | | | | | |
| Hemopexin | P02790 | LLQDEFPGIPSPLDAAVECHR | 117 | b5 | 836.8 | 890.5 | 60 | 401615 | 101128 | 25.2 | | | | | | |
| Hemopexin | P02790 | LLQDEFPGIPSPLDAAVECHR | 117 | b3 | 836.8 | 614.4 | 60 | 2548500 | 647127 | 25.4 | | | | | | |
| Hemopexin | P02790 | NFPSPVDAAFR | 113 | y7 | 680.9 | 775.4 | 52 | 1542332 | 231787 | 15.0 | 0.94 | 0.03 | 2.9 | 1.10 | 0.11 | 10.4 |
| Hemopexin | P02790 | NFPSPVDAAFR | 113 | y5 | 680.9 | 579.3 | 52 | 1075669 | 182822 | 17.0 | 1.10 | 0.03 | 2.5 | | | |
| Hemopexin | P02790 | NFPSPVDAAFR | 113 | b4 | 680.9 | 586.3 | 52 | 666526 | 102684 | 15.4 | 1.19 | 0.03 | 2.8 | | | |
| Hemopexin | P02790 | NFPSPVDAAFR | 113 | y8 | 680.9 | 862.4 | 52 | 576110 | 87491 | 15.2 | 1.17 | 0.03 | 2.7 | | | |
| Hemopexin | P02790 | NFPSPVDAAFR | 117 | y7 | 682.9 | 775.4 | 52 | 1646294 | 260488 | 15.8 | | | | | | |
| Hemopexin | P02790 | NFPSPVDAAFR | 117 | y5 | 682.9 | 579.3 | 52 | 974460 | 152010 | 15.6 | | | | | | |
| Hemopexin | P02790 | NFPSPVDAAFR | 117 | b4 | 682.9 | 590.3 | 52 | 559251 | 90608 | 16.2 | | | | | | |
| Hemopexin | P02790 | NFPSPVDAAFR | 117 | y8 | 682.9 | 862.4 | 52 | 494815 | 78938 | 16.0 | | | | | | |
| Hemopexin | P02790 | VDGALCMEK | 113 | b4 | 434.9 | 483.3 | 40 | 365675 | 97842 | 26.8 | 1.12 | 0.07 | 6.3 | 1.29 | 0.11 | 8.9 |
| Hemopexin | P02790 | VDGALCMEK | 113 | y3 | 651.8 | 547.3 | 51 | 521341 | 196861 | 37.8 | 1.34 | 0.11 | 8.5 | | | |
| Hemopexin | P02790 | VDGALCMEK | 113 | b5 | 651.8 | 596.3 | 51 | 229333 | 80668 | 35.2 | 1.36 | 0.09 | 6.7 | | | |
| Hemopexin | P02790 | VDGALCMEK | 113 | y4 | 651.8 | 707.3 | 51 | 467714 | 170900 | 36.5 | 1.33 | 0.13 | 9.8 | | | |
| Hemopexin | P02790 | VDGALCMEK | 117 | b4 | 437.6 | 487.3 | 40 | 327041 | 86052 | 26.3 | | | | | | |
| Hemopexin | P02790 | VDGALCMEK | 117 | y3 | 655.8 | 551.3 | 51 | 388433 | 135934 | 35.0 | | | | | | |
| Hemopexin | P02790 | VDGALCMEK | 117 | b5 | 655.8 | 600.3 | 51 | 167248 | 56335 | 33.7 | | | | | | |
| Hemopexin | P02790 | VDGALCMEK | 117 | y4 | 655.8 | 711.3 | 51 | 356035 | 129849 | 36.5 | | | | | | |
| Hemopexin | P02790 | YYCFQGNQFLR | 113 | Y7 | 818.4 | 862.4 | 59 | 253586 | 25045 | 9.9 | 0.86 | 0.02 | 2.3 | 1.06 | 0.14 | 13.4 |
| Hemopexin | P02790 | YYCFQGNQFLR | 113 | B5 | 818.4 | 902.4 | 59 | 96045 | 7663 | 8.0 | 1.06 | 0.06 | 6.1 | | | |
| Hemopexin | P02790 | YYCFQGNQFLR | 113 | Y9 | 818.4 | 1169.6 | 59 | 410258 | 46036 | 11.2 | 1.15 | 0.05 | 4.5 | | | |
| Hemopexin | P02790 | YYCFQGNQFLR | 113 | Y10 | 818.4 | 1332.6 | 59 | 316181 | 38953 | 12.3 | 1.17 | 0.06 | 5.2 | | | |
| Hemopexin | P02790 | YYCFQGNQFLR | 117 | Y7 | 820.4 | 862.4 | 59 | 295507 | 31936 | 10.8 | | | | | | |
| Hemopexin | P02790 | YYCFQGNQFLR | 117 | B5 | 820.4 | 906.4 | 59 | 91614 | 12475 | 13.6 | | | | | | |
| Hemopexin | P02790 | YYCFQGNQFLR | 117 | Y9 | 820.4 | 1169.6 | 59 | 358312 | 41173 | 11.5 | | | | | | |
| Hemopexin | P02790 | YYCFQGNQFLR | 117 | Y10 | 820.4 | 1332.6 | 59 | 269209 | 27179 | 10.1 | | | | | | |
| Heparin cofactor 2 | P05546 | IAIDLFK | 113 | y3 | 550.4 | 547.4 | 46 | 60644 | 7979 | 13.2 | 0.89 | 0.14 | 15.4 | 0.99 | 0.17 | 17.0 |
| Heparin cofactor 2 | P05546 | IAIDLFK | 113 | b4 | 550.4 | 553.3 | 46 | 54116 | 7576 | 14.0 | 1.02 | 0.09 | 8.6 | | | |
| Heparin cofactor 2 | P05546 | IAIDLFK | 113 | y4 | 550.4 | 662.4 | 46 | 16671 | 3408 | 20.4 | 0.83 | 0.19 | 23.5 | | | |
| Heparin cofactor 2 | P05546 | IAIDLFK | 113 | y6 | 550.4 | 846.5 | 46 | 5677 | 1349 | 23.8 | 1.21 | 0.26 | 21.8 | | | |
| Heparin cofactor 2 | P05546 | IAIDLFK | 117 | y3 | 554.4 | 551.4 | 46 | 69074 | 11170 | 16.2 | | | | | | |
| Heparin cofactor 2 | P05546 | IAIDLFK | 117 | b4 | 554.4 | 557.3 | 46 | 53566 | 8395 | 15.7 | | | | | | |
| Heparin cofactor 2 | P05546 | IAIDLFK | 117 | y4 | 554.4 | 666.4 | 46 | 20396 | 3447 | 16.9 | | | | | | |
| Heparin cofactor 2 | P05546 | IAIDLFK | 117 | y6 | 554.4 | 850.5 | 46 | 4741 | 868 | 18.3 | | | | | | |
| Heparin cofactor 2 | P05546 | NYNLVESLK | 113 | y3 | 680.4 | 487.3 | 52 | 163975 | 7371 | 4.5 | 1.02 | 0.07 | 7.0 | 1.19 | 0.14 | 11.9 |
| Heparin cofactor 2 | P05546 | NYNLVESLK | 113 | b3 | 680.4 | 532.3 | 52 | 160378 | 8992 | 5.6 | 1.16 | 0.07 | 6.2 | | | |
| Heparin cofactor 2 | P05546 | NYNLVESLK | 113 | y4 | 680.4 | 616.4 | 52 | 53528 | 5991 | 11.2 | 1.37 | 0.21 | 15.0 | | | |
| Heparin cofactor 2 | P05546 | NYNLVESLK | 113 | y5 | 680.4 | 715.4 | 52 | 27455 | 2410 | 8.8 | 1.20 | 0.11 | 9.1 | | | |
| Heparin cofactor 2 | P05546 | NYNLVESLK | 117 | y | 684.4 | 491.3 | 52 | 160647 | 12561 | 7.8 | | | | | | |
| Heparin cofactor 2 | P05546 | NYNLVESLK | 117 | b | 684.4 | 536.3 | 52 | 138255 | 6256 | 4.5 | | | | | | |
| Heparin cofactor 2 | P05546 | NYNLVESLK | 117 | y | 684.4 | 620.4 | 52 | 39429 | 3805 | 9.7 | | | | | | |
| Heparin cofactor 2 | P05546 | NYNLVESLK | 117 | y | 684.4 | 719.4 | 52 | 23001 | 2905 | 12.6 | | | | | | |
| Heparin cofactor 2 | P05546 | TLEAQLTPR | 113 | b3 | 584.8 | 484.3 | 47 | 197608 | 21300 | 10.8 | 1.19 | 0.07 | 5.6 | 1.29 | 0.11 | 8.8 |
| Heparin cofactor 2 | P05546 | TLEAQLTPR | 113 | y6 | 584.8 | 685.4 | 47 | 101996 | 9657 | 9.5 | 1.31 | 0.07 | 5.2 | | | |
| Heparin cofactor 2 | P05546 | TLEAQLTPR | 113 | b5 | 584.8 | 683.4 | 47 | 87765 | 10115 | 11.5 | 1.45 | 0.10 | 6.9 | | | |
| Heparin cofactor 2 | P05546 | TLEAQLTPR | 113 | y8 | 584.8 | 927.5 | 47 | 33685 | 4636 | 13.8 | 1.23 | 0.11 | 8.9 | | | |
| Heparin cofactor 2 | P05546 | TLEAQLTPR | 117 | b3 | 586.8 | 488.3 | 47 | 166044 | 16786 | 10.1 | | | | | | |
| Heparin cofactor 2 | P05546 | TLEAQLTPR | 117 | y6 | 586.8 | 685.4 | 47 | 77836 | 7352 | 9.4 | | | | | | |
| Heparin cofactor 2 | P05546 | TLEAQLTPR | 117 | b5 | 586.8 | 687.4 | 47 | 60778 | 7129 | 11.7 | | | | | | |
| Heparin cofactor 2 | P05546 | TLEAQLTPR | 117 | y8 | 586.8 | 927.5 | 47 | 27434 | 3263 | 11.9 | | | | | | |
| Histidine-rich gprot. | P04196 | DGYLFQLLR | 113 | b3 | 632.9 | 476.2 | 50 | 902418 | 45414 | 5.0 | 0.97 | 0.01 | 1.0 | | | |
| Histidine-rich gprot. | P04196 | DGYLFQLLR | 113 | b4 | 632.9 | 589.3 | 50 | 370123 | 21842 | 5.9 | 0.95 | 0.02 | 1.9 | | | |
| Histidine-rich gprot. | P04196 | DGYLFQLLR | 113 | y5 | 632.9 | 676.4 | 50 | 88688 | 7291 | 8.2 | 1.03 | 0.08 | 7.4 | | | |
| Histidine-rich gprot. | P04196 | DGYLFQLLR | 113 | y6 | 632.9 | 789.5 | 50 | 49738 | 3920 | 7.9 | 1.05 | 0.09 | 8.2 | | | |
| Histidine-rich gprot. | P04196 | DGYLFQLLR | 117 | b3 | 634.9 | 480.2 | 50 | 927290 | 45238 | 4.9 | | | | | | |
| Histidine-rich gprot. | P04196 | DGYLFQLLR | 117 | b4 | 634.9 | 593.3 | 50 | 388844 | 21775 | 5.6 | | | | | | |
| Histidine-rich gprot. | P04196 | DGYLFQLLR | 117 | y5 | 634.9 | 676.4 | 50 | 86484 | 4836 | 5.6 | | | | | | |
| Histidine-rich gprot. | P04196 | DGYLFQLLR | 117 | y6 | 634.9 | 789.5 | 50 | 47475 | 3792 | 8.0 | | | | | | |
| Histidine-rich gprot. | P04196 | DSPVLIDFFEDTER | 113 | b4 | 608.3 | 539.3 | 48 | 199232 | 47976 | 24.1 | 1.42 | 0.11 | 7.9 | 1.15 | 0.19 | 16.5 |
| Histidine-rich gprot. | P04196 | DSPVLIDFFEDTER | 113 | b4 | 911.9 | 539.3 | 64 | 177914 | 46409 | 26.1 | 1.14 | 0.07 | 6.4 | | | |
| Histidine-rich gprot. | P04196 | DSPVLIDFFEDTER | 113 | y5 | 608.3 | 649.3 | 48 | 26494 | 6854 | 25.9 | 1.08 | 0.16 | 15.0 | | | |
| Histidine-rich gprot. | P04196 | DSPVLIDFFEDTER | 113 | y7 | 911.9 | 943.4 | 64 | 69208 | 21711 | 31.4 | 0.97 | 0.07 | 7.5 | | | |
| Histidine-rich gprot. | P04196 | DSPVLIDFFEDTER | 117 | b4 | 609.6 | 543.3 | 48 | 140648 | 33007 | 23.5 | | | | | | |
| Histidine-rich gprot. | P04196 | DSPVLIDFFEDTER | 117 | b4 | 913.9 | 543.3 | 64 | 157062 | 44396 | 28.3 | | | | | | |
| Histidine-rich gprot. | P04196 | DSPVLIDFFEDTER | 117 | y5 | 609.6 | 649.3 | 48 | 24796 | 6832 | 27.6 | | | | | | |
| Histidine-rich gprot. | P04196 | DSPVLIDFFEDTER | 117 | y7 | 913.9 | 943.4 | 64 | 72228 | 26200 | 36.3 | | | | | | |
| Histidine-rich gprot. | P04196 | GGEGTGYFVDFSVR | 113 | y4 | 815.9 | 508.3 | 59 | 266901 | 58453 | 21.9 | 0.80 | 0.05 | 6.2 | 0.88 | 0.13 | 14.7 |
| Histidine-rich gprot. | P04196 | GGEGTGYFVDFSVR | 113 | b6 | 815.9 | 599.3 | 59 | 81781 | 22265 | 27.2 | 0.97 | 0.11 | 11.2 | | | |
| Histidine-rich gprot. | P04196 | GGEGTGYFVDFSVR | 113 | b7 | 815.9 | 762.3 | 59 | 71228 | 16886 | 23.7 | 1.02 | 0.08 | 8.1 | | | |
| Histidine-rich gprot. | P04196 | GGEGTGYFVDFSVR | 113 | y6 | 815.9 | 722.4 | 59 | 18128 | 4207 | 23.2 | 0.75 | 0.12 | 15.6 | | | |
| Histidine-rich gprot. | P04196 | GGEGTGYFVDFSVR | 117 | y4 | 817.9 | 508.3 | 59 | 336584 | 82634 | 24.6 | | | | | | |
| Histidine-rich gprot. | P04196 | GGEGTGYFVDFSVR | 117 | b6 | 817.9 | 603.3 | 59 | 83971 | 20246 | 24.1 | | | | | | |
| Histidine-rich gprot. | P04196 | GGEGTGYFVDFSVR | 117 | b7 | 817.9 | 766.3 | 59 | 70553 | 17669 | 25.0 | | | | | | |
| Histidine-rich gprot. | P04196 | GGEGTGYFVDFSVR | 117 | y6 | 817.9 | 722.4 | 59 | 24856 | 6929 | 27.9 | | | | | | |
| IAT IHC H1 | P19827 | ELAAQTIK | 113 | b3 | 577.4 | 454.3 | 47 | 216935 | 75067 | 34.6 | 1.09 | 0.13 | 11.8 | 1.05 | 0.07 | 6.8 |
| IAT IHC H1 | P19827 | ELAAQTIK | 113 | y3 | 577.4 | 501.3 | 47 | 89643 | 37070 | 41.4 | 1.04 | 0.10 | 9.8 | | | |
| IAT IHC H1 | P19827 | ELAAQTIK | 113 | b4 | 577.4 | 525.3 | 47 | 84869 | 29946 | 35.3 | 1.13 | 0.08 | 6.7 | | | |
| IAT IHC H1 | P19827 | ELAAQTIK | 113 | y5 | 577.4 | 700.4 | 47 | 22969 | 9322 | 40.6 | 0.96 | 0.10 | 10.4 | | | |
| IAT IHC H1 | P19827 | ELAAQTIK | 117 | b3 | 581.4 | 458.3 | 47 | 201674 | 74538 | 37.0 | | | | | | |
| IAT IHC H1 | P19827 | ELAAQTIK | 117 | y3 | 581.4 | 505.3 | 47 | 85655 | 31920 | 37.3 | | | | | | |
| IAT IHC H1 | P19827 | ELAAQTIK | 117 | b4 | 581.4 | 529.3 | 47 | 75454 | 26416 | 35.0 | | | | | | |
| IAT IHC H1 | P19827 | ELAAQTIK | 117 | y5 | 581.4 | 704.4 | 47 | 23917 | 8922 | 37.3 | | | | | | |
| IAT IHC H1 | P19827 | FAHYVVTSQVVNTANEAR | 113 | b3 | 716.1 | 496.3 | 54 | 815485 | 73870 | 9.1 | 1.13 | 0.03 | 2.7 | 1.23 | 0.25 | 20.2 |
| IAT IHC H1 | P19827 | FAHYVVTSQVVNTANEAR | 113 | b4 | 716.1 | 659.3 | 54 | 259251 | 25200 | 9.7 | 1.20 | 0.06 | 4.8 | | | |
| IAT IHC H1 | P19827 | FAHYVVTSQVVNTANEAR | 113 | b5 | 716.1 | 758.4 | 54 | 196960 | 21462 | 10.9 | 1.59 | 0.08 | 5.3 | | | |
| IAT IHC H1 | P19827 | FAHYVVTSQVVNTANEAR | 113 | y7 | 716.1 | 775.4 | 54 | 143311 | 11896 | 8.3 | 1.01 | 0.05 | 5.3 | | | |
| IAT IHC H1 | P19827 | FAHYVVTSQVVNTANEAR | 117 | b3 | 717.4 | 500.3 | 54 | 723340 | 72451 | 10.0 | | | | | | |
| IAT IHC H1 | P19827 | FAHYVVTSQVVNTANEAR | 117 | b4 | 717.4 | 663.3 | 54 | 216466 | 27598 | 12.7 | | | | | | |
| IAT IHC H1 | P19827 | FAHYVVTSQVVNTANEAR | 117 | b5 | 717.4 | 762.4 | 54 | 124344 | 12945 | 10.4 | | | | | | |
| IAT IHC H1 | P19827 | FAHYVVTSQVVNTANEAR | 117 | y7 | 717.4 | 775.4 | 54 | 142591 | 16382 | 11.5 | | | | | | |
| IAT IHC H1 | P19827 | LDAQASFLPK | 113 | b3 | 685.4 | 440.3 | 52 | 335950 | 99599 | 29.6 | 1.15 | 0.05 | 4.2 | 1.10 | 0.11 | 9.8 |
| IAT IHC H1 | P19827 | LDAQASFLPK | 113 | b4 | 685.4 | 568.3 | 52 | 319177 | 85224 | 26.7 | 1.16 | 0.06 | 5.2 | | | |
| IAT IHC H1 | P19827 | LDAQASFLPK | 113 | b5 | 685.4 | 639.3 | 52 | 137827 | 31972 | 23.2 | 0.94 | 0.07 | 7.4 | | | |
| IAT IHC H1 | P19827 | LDAQASFLPK | 113 | b6 | 685.4 | 726.4 | 52 | 77352 | 17997 | 23.3 | 1.15 | 0.08 | 6.6 | | | |
| IAT IHC H1 | P19827 | LDAQASFLPK | 117 | b3 | 689.4 | 444.3 | 52 | 292877 | 87975 | 30.0 | | | | | | |
| IAT IHC H1 | P19827 | LDAQASFLPK | 117 | b4 | 689.4 | 572.3 | 52 | 275519 | 73885 | 26.8 | | | | | | |
| IAT IHC H1 | P19827 | LDAQASFLPK | 117 | b5 | 689.4 | 643.4 | 52 | 149045 | 39930 | 26.8 | | | | | | |
| IAT IHC H1 | P19827 | LDAQASFLPK | 117 | b6 | 689.4 | 730.4 | 52 | 67909 | 17308 | 25.5 | | | | | | |
| IAT IHC H2 | P19823 | NDLISATK | 113 | y4 | 571.3 | 546.3 | 47 | 195946 | 41358 | 21.1 | 1.08 | 0.07 | 6.7 | 1.08 | 0.05 | 4.8 |
| IAT IHC H2 | P19823 | NDLISATK | 113 | b3 | 571.3 | 483.3 | 47 | 223165 | 36742 | 16.5 | 1.10 | 0.03 | 2.9 | | | |
| IAT IHC H2 | P19823 | NDLISATK | 113 | y3 | 571.3 | 459.3 | 47 | 129868 | 21670 | 16.7 | 1.00 | 0.08 | 8.3 | | | |
| IAT IHC H2 | P19823 | NDLISATK | 113 | y5 | 571.3 | 659.4 | 47 | 43141 | 9357 | 21.7 | 1.12 | 0.08 | 6.9 | | | |
| IAT IHC H2 | P19823 | NDLISATK | 117 | y4 | 575.3 | 550.3 | 47 | 180406 | 33937 | 18.8 | | | | | | |
| IAT IHC H2 | P19823 | NDLISATK | 117 | b3 | 575.3 | 487.3 | 47 | 202865 | 32373 | 16.0 | | | | | | |
| IAT IHC H2 | P19823 | NDLISATK | 117 | y3 | 575.3 | 463.3 | 47 | 129812 | 20157 | 15.5 | | | | | | |
| IAT IHC H2 | P19823 | NDLISATK | 117 | y5 | 575.3 | 663.4 | 47 | 38613 | 8807 | 22.8 | | | | | | |
| IAT IHC H2 | P19823 | SLAPTAAAK | 113 | y3 | 555.3 | 429.3 | 46 | 154931 | 59225 | 38.2 | 1.09 | 0.09 | 8.6 | 1.15 | 0.07 | 6.3 |
| IAT IHC H2 | P19823 | SLAPTAAAK | 113 | b3 | 555.3 | 412.3 | 46 | 137046 | 54926 | 40.1 | 1.25 | 0.13 | 10.4 | | | |
| IAT IHC H2 | P19823 | SLAPTAAAK | 113 | y4 | 555.3 | 500.3 | 46 | 95295 | 36190 | 38.0 | 1.17 | 0.15 | 12.8 | | | |
| IAT IHC H2 | P19823 | SLAPTAAAK | 113 | y6 | 555.3 | 698.4 | 46 | 64288 | 27718 | 43.1 | 1.10 | 0.16 | 14.4 | | | |
| IAT IHC H2 | P19823 | SLAPTAAAK | 117 | y3 | 559.3 | 433.3 | 46 | 143099 | 54528 | 38.1 | | | | | | |
| IAT IHC H2 | P19823 | SLAPTAAAK | 117 | b3 | 559.3 | 416.3 | 46 | 111101 | 46680 | 42.0 | | | | | | |
| IAT IHC H2 | P19823 | SLAPTAAAK | 117 | y4 | 559.3 | 504.3 | 46 | 83509 | 35358 | 42.3 | | | | | | |
| IAT IHC H2 | P19823 | SLAPTAAAK | 117 | y6 | 559.3 | 702.4 | 46 | 60660 | 30183 | 49.8 | | | | | | |
| IAT IHC H2 | P19823 | SSALDMENFR | 113 | y5 | 655.3 | 696.3 | 51 | 266028 | 21987 | 8.3 | 0.82 | 0.03 | 3.6 | 0.96 | 0.10 | 9.9 |
| IAT IHC H2 | P19823 | SSALDMENFR | 113 | b3 | 655.3 | 386.2 | 51 | 283986 | 30433 | 10.7 | 1.02 | 0.06 | 6.3 | | | |
| IAT IHC H2 | P19823 | SSALDMENFR | 113 | b5 | 655.3 | 614.3 | 51 | 206874 | 20600 | 10.0 | 1.01 | 0.05 | 4.5 | | | |
| IAT IHC H2 | P19823 | SSALDMENFR | 113 | y6 | 655.3 | 811.3 | 51 | 36536 | 3453 | 9.5 | 0.99 | 0.07 | 6.9 | | | |
| IAT IHC H2 | P19823 | SSALDMENFR | 117 | y5 | 657.3 | 696.3 | 51 | 325169 | 33126 | 10.2 | | | | | | |
| IAT IHC H2 | P19823 | SSALDMENFR | 117 | b3 | 657.3 | 390.2 | 51 | 277841 | 27196 | 9.8 | | | | | | |
| IAT IHC H2 | P19823 | SSALDMENFR | 117 | b5 | 657.3 | 618.3 | 51 | 204617 | 24793 | 12.1 | | | | | | |
| IAT IHC H2 | P19823 | SSALDMENFR | 117 | y6 | 657.3 | 811.3 | 51 | 37173 | 5012 | 13.5 | | | | | | |
| IAT IHC H2 | P19823 | TEVNVLPGAK | 113 | y4 | 654.4 | 512.3 | 51 | 1707516 | 212666 | 12.5 | 1.22 | 0.04 | 3.2 | 1.21 | 0.02 | 1.9 |
| IAT IHC H2 | P19823 | TEVNVLPGAK | 113 | b4 | 654.4 | 584.3 | 51 | 686456 | 86094 | 12.5 | 1.22 | 0.06 | 5.1 | | | |
| IAT IHC H2 | P19823 | TEVNVLPGAK | 113 | b5 | 654.4 | 683.4 | 51 | 173780 | 28623 | 16.5 | 1.22 | 0.09 | 7.3 | | | |
| IAT IHC H2 | P19823 | TEVNVLPGAK | 113 | y3 | 654.4 | 415.3 | 51 | 551354 | 36988 | 6.7 | 1.17 | 0.05 | 4.7 | | | |
| IAT IHC H2 | P19823 | TEVNVLPGAK | 117 | y4 | 658.4 | 516.3 | 51 | 1405725 | 198079 | 14.1 | | | | | | |
| IAT IHC H2 | P19823 | TEVNVLPGAK | 117 | b4 | 658.4 | 588.3 | 51 | 562863 | 47412 | 8.4 | | | | | | |
| IAT IHC H2 | P19823 | TEVNVLPGAK | 117 | b5 | 658.4 | 687.4 | 51 | 142730 | 25659 | 18.0 | | | | | | |
| IAT IHC H2 | P19823 | TEVNVLPGAK | 117 | y3 | 658.4 | 419.3 | 51 | 470570 | 36122 | 7.7 | | | | | | |
| IAT IHC H2 | P19823 | VQFELHYQEVK | 113 | b3 | 567.3 | 515.3 | 46 | 306177 | 53345 | 17.4 | 0.99 | 0.06 | 5.9 | 1.00 | 0.08 | 7.8 |
| IAT IHC H2 | P19823 | VQFELHYQEVK | 113 | b4 | 567.3 | 644.3 | 46 | 347835 | 59278 | 17.0 | 1.03 | 0.04 | 3.8 | | | |
| IAT IHC H2 | P19823 | VQFELHYQEVK | 113 | y4 | 567.3 | 643.4 | 46 | 43802 | 7384 | 16.9 | 0.90 | 0.10 | 11.2 | | | |
| IAT IHC H2 | P19823 | VQFELHYQEVK | 113 | b5 | 567.3 | 757.4 | 46 | 55415 | 12302 | 22.2 | 1.09 | 0.09 | 8.1 | | | |
| IAT IHC H2 | P19823 | VQFELHYQEVK | 117 | b3 | 570.0 | 519.3 | 46 | 309899 | 62724 | 20.2 | | | | | | |
| IAT IHC H2 | P19823 | VQFELHYQEVK | 117 | b4 | 570.0 | 648.3 | 46 | 338667 | 64925 | 19.2 | | | | | | |
| IAT IHC H2 | P19823 | VQFELHYQEVK | 117 | y4 | 570.0 | 647.4 | 46 | 49710 | 12444 | 25.0 | | | | | | |
| IAT IHC H2 | P19823 | VQFELHYQEVK | 117 | b5 | 570.0 | 761.4 | 46 | 50702 | 9127 | 18.0 | | | | | | |
| IAT IHC H2 | P19823 | VVNNSPQPQNVVFDVQIPK | 113 | b3 | 801.4 | 453.3 | 58 | 94186 | 23854 | 25.3 | 1.16 | 0.08 | 6.7 | 1.16 | 0.05 | 4.4 |
| IAT IHC H2 | P19823 | VVNNSPQPQNVVFDVQIPK | 113 | b5 | 801.4 | 654.4 | 58 | 111523 | 34562 | 31.0 | 1.21 | 0.06 | 4.8 | | | |
| IAT IHC H2 | P19823 | VVNNSPQPQNVVFDVQIPK | 113 | b4 | 801.4 | 567.3 | 58 | 93024 | 29796 | 32.0 | 1.19 | 0.09 | 7.6 | | | |
| IAT IHC H2 | P19823 | VVNNSPQPQNVVFDVQIPK | 113 | b7 | 801.4 | 879.5 | 58 | 37807 | 12056 | 31.9 | 1.09 | 0.09 | 8.4 | | | |
| IAT IHC H2 | P19823 | VVNNSPQPQNVVFDVQIPK | 117 | b3 | 804.1 | 457.3 | 58 | 81573 | 21560 | 26.4 | | | | | | |
| IAT IHC H2 | P19823 | VVNNSPQPQNVVFDVQIPK | 117 | b5 | 804.1 | 658.4 | 58 | 92075 | 27610 | 30.0 | | | | | | |
| IAT IHC H2 | P19823 | VVNNSPQPQNVVFDVQIPK | 117 | b4 | 804.1 | 571.3 | 58 | 76829 | 19865 | 25.9 | | | | | | |
| IAT IHC H2 | P19823 | VVNNSPQPQNVVFDVQIPK | 117 | b7 | 804.1 | 883.5 | 58 | 34330 | 9765 | 28.4 | | | | | | |
| IAT IHC H4 | Q14624 | GSEMVVAGK | 113 | b3 | 579.3 | 414.2 | 47 | 458137 | 144108 | 31.5 | 0.86 | 0.04 | 5.1 | 0.88 | 0.03 | 3.9 |
| IAT IHC H4 | Q14624 | GSEMVVAGK | 113 | y3 | 579.3 | 415.3 | 47 | 251415 | 84951 | 33.8 | 0.86 | 0.05 | 5.4 | | | |
| IAT IHC H4 | Q14624 | GSEMVVAGK | 113 | y4 | 579.3 | 514.3 | 47 | 97720 | 36060 | 36.9 | 0.89 | 0.04 | 4.0 | | | |
| IAT IHC H4 | Q14624 | GSEMVVAGK | 113 | b4 | 579.3 | 545.2 | 47 | 200380 | 65457 | 32.7 | 0.93 | 0.05 | 5.7 | | | |
| IAT IHC H4 | Q14624 | GSEMVVAGK | 117 | b3 | 583.3 | 418.2 | 47 | 532674 | 150624 | 28.3 | | | | | | |
| IAT IHC H4 | Q14624 | GSEMVVAGK | 117 | y3 | 583.3 | 419.3 | 47 | 293834 | 97561 | 33.2 | | | | | | |
| IAT IHC H4 | Q14624 | GSEMVVAGK | 117 | y4 | 583.3 | 518.3 | 47 | 110838 | 42615 | 38.4 | | | | | | |
| IAT IHC H4 | Q14624 | GSEMVVAGK | 117 | b4 | 583.3 | 549.2 | 47 | 216778 | 72520 | 33.5 | | | | | | |
| IAT IHC H4 | Q14624 | ILDDLSPR | 113 | y5 | 534.8 | 587.3 | 45 | 134277 | 32914 | 24.5 | 1.00 | 0.03 | 3.2 | 1.13 | 0.13 | 11.4 |
| IAT IHC H4 | Q14624 | ILDDLSPR | 113 | b4 | 534.8 | 597.3 | 45 | 193402 | 51167 | 26.5 | 1.14 | 0.08 | 7.4 | | | |
| IAT IHC H4 | Q14624 | ILDDLSPR | 113 | b3 | 534.8 | 482.3 | 45 | 174131 | 48300 | 27.7 | 1.26 | 0.08 | 6.7 | | | |
| IAT IHC H4 | Q14624 | ILDDLSPR | 113 | y3 | 534.8 | 359.2 | 45 | | | | | | | | | |
| IAT IHC H4 | Q14624 | ILDDLSPR | 117 | y5 | 536.8 | 587.3 | 45 | 133110 | 31011 | 23.3 | | | | | | |
| IAT IHC H4 | Q14624 | ILDDLSPR | 117 | b4 | 536.8 | 601.3 | 45 | 169412 | 42589 | 25.1 | | | | | | |
| IAT IHC H4 | Q14624 | ILDDLSPR | 117 | b3 | 536.8 | 486.3 | 45 | 137695 | 36850 | 26.8 | | | | | | |
| IAT IHC H4 | Q14624 | ILDDLSPR | 117 | y3 | 536.8 | 359.2 | 45 | | | | | | | | | |
| IAT IHC H4 | Q14624 | NVVFVIDK | 113 | b3 | 607.4 | 453.3 | 48 | 346104 | 74082 | 21.4 | 1.31 | 0.07 | 5.7 | 1.37 | 0.17 | 12.5 |
| IAT IHC H4 | Q14624 | NVVFVIDK | 113 | y4 | 607.4 | 614.4 | 48 | 96360 | 16936 | 17.6 | 1.31 | 0.12 | 8.8 | | | |
| IAT IHC H4 | Q14624 | NVVFVIDK | 113 | y5 | 607.4 | 761.5 | 48 | 74079 | 12261 | 16.6 | 1.63 | 0.10 | 6.2 | | | |
| IAT IHC H4 | Q14624 | NVVFVIDK | 113 | b4 | 607.4 | 600.4 | 48 | 102015 | 18319 | 18.0 | 1.24 | 0.06 | 5.0 | | | |
| IAT IHC H4 | Q14624 | NVVFVIDK | 117 | b3 | 611.4 | 457.3 | 49 | 265903 | 65209 | 24.5 | | | | | | |
| IAT IHC H4 | Q14624 | NVVFVIDK | 117 | y4 | 611.4 | 618.4 | 49 | 73868 | 14438 | 19.5 | | | | | | |
| IAT IHC H4 | Q14624 | NVVFVIDK | 117 | y5 | 611.4 | 765.5 | 49 | 45733 | 8106 | 17.7 | | | | | | |
| IAT IHC H4 | Q14624 | NVVFVIDK | 117 | b4 | 611.4 | 604.4 | 49 | 82543 | 16107 | 19.5 | | | | | | |
| Kininogen-1 | P01095 | DIPTNSPELEETLTHTITK | 113 | y3 | 807.1 | 501.3 | 58 | 641083 | 83847 | 13.1 | 1.10 | 0.03 | 3.1 | 1.15 | 0.08 | 6.9 |
| Kininogen-1 | P01095 | DIPTNSPELEETLTHTITK | 113 | y4 | 807.1 | 602.4 | 58 | 639353 | 84858 | 13.3 | 1.09 | 0.05 | 4.5 | | | |
| Kininogen-1 | P01095 | DIPTNSPELEETLTHTITK | 113 | y6 | 807.1 | 840.5 | 58 | 161236 | 20298 | 12.6 | 1.15 | 0.07 | 6.5 | | | |
| Kininogen-1 | P01093 | DIPTNSPELEETLTHTITK | 113 | b5 | 807.1 | 681.4 | 58 | 348857 | 44388 | 12.7 | 1.26 | 0.07 | 5.6 | | | |
| Kininogen-1 | P01095 | DIPTNSPELEETLTHTITK | 117 | y3 | 809.8 | 505.3 | 58 | 583514 | 72016 | 12.3 | | | | | | |
| Kininogen-i | P01095 | DIPTNSPELEETLTHTITK | 117 | y4 | 809.8 | 606.4 | 58 | 586799 | 82082 | 14.0 | | | | | | |
| Kininogen-1 | P01095 | DIPTNSPELEETLTHTITK | 117 | y6 | 809.8 | 844.5 | 58 | 140704 | 19717 | 14.0 | | | | | | |
| Kininogen-1 | P01093 | DIPTNSPELEETLTHTITK | 117 | b5 | 809.8 | 685.5 | 58 | 277133 | 37997 | 13.7 | | | | | | |
| Kininogen-1 | P01042 | ENFLFLTPDCK | 113 | b3 | 555.3 | 531.3 | 46 | 1128007 | 200606 | 17.8 | 1.12 | 0.03 | 2.7 | 1.10 | 0.06 | 5.3 |
| Kininogen-1 | P01042 | ENFLFLTPDCK | 113 | b3 | 832.4 | 531.3 | 60 | 387309 | 102525 | 26.5 | 1.02 | 0.03 | 3.1 | | | |
| Kininogen-1 | P01042 | ENFLFLTPDCK | 113 | y3 | 555.3 | 562.3 | 46 | 192786 | 31110 | 16.1 | 1.16 | 0.06 | 5.5 | | | |
| Kininogen-1 | P01042 | ENFLFLTPDCK | 113 | b4 | 555.3 | 644.3 | 46 | 268055 | 48046 | 17.9 | 1.10 | 0.07 | 6.1 | | | |
| Kininogen-1 | P01042 | ENFLFLTPDCK | 117 | b3 | 558.0 | 535.3 | 46 | 1004094 | 171851 | 17.1 | | | | | | |
| Kininogen-1 | P01042 | ENFLFLTPDCK | 117 | b3 | 836.4 | 535.3 | 60 | 378352 | 95823 | 25.3 | | | | | | |
| Kininogen-1 | P01042 | ENFLFLTPDCK | 117 | y3 | 558.0 | 566.3 | 46 | 167345 | 30352 | 18.1 | | | | | | |
| Kininogen-1 | P01042 | ENFLFLTPDCK | 117 | b4 | 558.0 | 648.3 | 46 | 244102 | 46040 | 18.9 | | | | | | |
| Kininogen-1 | P01042 | TVGSDTFYSFK | 113 | y3 | 511.2 | 521.3 | 44 | 418766 | 58396 | 13.9 | 1.50 | 0.06 | 4.2 | 1.27 | 0.26 | 20.2 |
| Kininogen-1 | P01042 | TVGSDTFYSFK | 113 | y3 | 766.4 | 521.3 | 56 | 277104 | 39368 | 14.2 | 1.30 | 0.03 | 2.4 | | | |
| Kininogen-1 | P01042 | TVGSDTFYSFK | 113 | b5 | 511.2 | 600.3 | 44 | 318221 | 53929 | 16.9 | 0.91 | 0.04 | 4.6 | | | |
| Kininogen-1 | P01042 | TVGSDTFYSFK | 113 | y4 | 766.4 | 684.4 | 56 | 146363 | 17555 | 12.0 | 1.39 | 0.06 | 4.6 | | | |
| Kininogen-1 | P01042 | TVGSDTFYSFK | 117 | y3 | 513.9 | 525.3 | 44 | 279949 | 37267 | 13.3 | | | | | | |
| Kininogen-1 | P01042 | TVGSDTFYSFK | 117 | y3 | 770.4 | 525.3 | 57 | 213682 | 29415 | 13.8 | | | | | | |
| Kininogen-1 | P01042 | TVGSDTFYSFK | 117 | b5 | 513.9 | 604.3 | 44 | 351949 | 61072 | 17.4 | | | | | | |
| Kininogen-1 | P01042 | TVGSDTFYSFK | 117 | y4 | 770.4 | 688.4 | 57 | 105704 | 12916 | 12.2 | | | | | | |
| Kininogen-1 | P01042 | YFIDFVAR | 113 | b4 | 585.8 | 679.3 | 47 | 173528 | 50891 | 29.3 | 0.96 | 0.04 | 4.2 | 1.25 | 0.20 | 16.3 |
| Kininogen-1 | P01042 | YFIDFVAR | 113 | y5 | 585.8 | 607.3 | 47 | 74674 | 20913 | 28.0 | 1.35 | 0.20 | 14.9 | | | |
| Kininogen-1 | P01042 | YFIDFVAR | 113 | b3 | 585.8 | 564.3 | 47 | 50995 | 14564 | 28.6 | 1.43 | 0.16 | 11.5 | | | |
| Kininogen-1 | P01042 | YFIDFVAR | 113 | y7 | 585.8 | 867.5 | 47 | 36813 | 10799 | 29.3 | 1.26 | 0.13 | 10.0 | | | |
| Kininogen-1 | P01042 | YFIDFVAR | 117 | b4 | 587.8 | 683.4 | 47 | 180351 | 52194 | 28.9 | | | | | | |
| Kininogen-1 | P01042 | YFIDFVAR | 117 | y5 | 587.8 | 607.3 | 47 | 56960 | 20801 | 36.5 | | | | | | |
| Kininogen-1 | P01042 | YFIDFVAR | 117 | b3 | 587.8 | 568.3 | 47 | 36128 | 10560 | 29.2 | | | | | | |
| Kininogen-1 | P01042 | YFIDFVAR | 117 | y7 | 587.8 | 867.5 | 47 | 29650 | 8958 | 30.2 | | | | | | |
| Plasminogen | P00747 | EAQLPVIENK | 113 | b3 | 710.9 | 469.2 | 54 | 1043820 | 498166 | 47.7 | 1.01 | 0.07 | 6.6 | 1.01 | 0.03 | 2.8 |
| Plasminogen | P00747 | EAQLPVIENK | 113 | y3 | 710.9 | 530.3 | 54 | 230017 | 107999 | 47.0 | 1.03 | 0.10 | 9.2 | | | |
| Plasminogen | P00747 | EAQLPVIENK | 113 | y4 | 710.9 | 643.4 | 54 | 193275 | 90958 | 47.1 | 0.97 | 0.09 | 9.0 | | | |
| Plasminogen | P00747 | EAQLPVIENK | 113 | b4 | 710.9 | 582.3 | 54 | 149093 | 70922 | 47.6 | 1.02 | 0.09 | 8.6 | | | |
| Plasminogen | P00747 | EAQLPVIENK | 117 | b3 | 714.9 | 473.2 | 54 | 1046019 | 515109 | 49.2 | | | | | | |
| Plasminogen | P00747 | EAQLPVIENK | 117 | y3 | 714.9 | 534.3 | 54 | 223574 | 105730 | 47.3 | | | | | | |
| Plasminogen | P00747 | EAQLPVIENK | 117 | y4 | 714.9 | 647.4 | 54 | 199216 | 90455 | 45.4 | | | | | | |
| Plasminogen | P00747 | EAQLPVIENK | 117 | b4 | 714.9 | 586.3 | 54 | 146407 | 69642 | 47.6 | | | | | | |
| Plasminogen | P00747 | EPLDDYVNTQGASLFSVTK | 113 | b5 | 788.7 | 710.3 | 57 | 273252 | 129718 | 47.5 | 0.81 | 0.04 | 5.3 | 0.82 | 0.03 | 4.3 |
| Plasminogen | P00747 | EPLDDYVNTQGASLFSVTK | 113 | y4 | 788.7 | 574.4 | 57 | 121743 | 50224 | 41.3 | 0.83 | 0.05 | 6.1 | | | |
| Plasminogen | P00747 | EPLDDYVNTQGASLFSVTK | 113 | y5 | 788.7 | 721.4 | 57 | 79541 | 33101 | 41.6 | 0.85 | 0.06 | 7.5 | | | |
| Plasminogen | P00747 | EPLDDYVNTQGASLFSVTK | 113 | b6 | 788.7 | 873.4 | 57 | 86537 | 42757 | 49.4 | 0.77 | 0.06 | 7.6 | | | |
| Plasminogen | P00747 | EPLDDYVNTQGASLFSVTK | 117 | b5 | 791.4 | 714.3 | 58 | 332729 | 148111 | 44.5 | | | | | | |
| Plasminogen | P00747 | EPLDDYVNTQGASLFSVTK | 117 | y4 | 791.4 | 578.4 | 58 | 146302 | 57326 | 39.2 | | | | | | |
| Plasminogen | P00747 | EPLDDYVNTQGASLFSVTK | 117 | y5 | 791.4 | 725.4 | 58 | 93930 | 40401 | 43.0 | | | | | | |
| Plasminogen | P00747 | EPLDDYVNTQGASLFSVTK | 117 | b6 | 791.4 | 877.4 | 58 | 110450 | 49489 | 44.8 | | | | | | |
| Plasminogen | P00747 | LSSPAVITDK | 113 | y3 | 655.9 | 503.3 | 51 | 348749 | 100948 | 28.9 | 1.12 | 0.04 | 3.7 | 1.16 | 0.09 | 7.5 |
| Plasminogen | P00747 | LSSPAVITDK | 113 | y4 | 655.9 | 616.4 | 51 | 157570 | 40323 | 25.6 | 1.16 | 0.07 | 6.4 | | | |
| Plasminogen | P00747 | LSSPAVITDK | 113 | b5 | 655.9 | 596.3 | 51 | 147253 | 40993 | 27.8 | 1.09 | 0.07 | 6.1 | | | |
| Plasminogen | P00747 | LSSPAVITDK | 113 | y5 | 655.9 | 715.4 | 51 | 51592 | 15992 | 31.0 | 1.29 | 0.10 | 7.7 | | | |
| Plasminogen | P00747 | LSSPAVITDK | 117 | y3 | 659.9 | 507.3 | 51 | 311047 | 87676 | 28.2 | | | | | | |
| Plasminogen | P00747 | LSSPAVITDK | 117 | y4 | 659.9 | 620.4 | 51 | 137083 | 37239 | 27.2 | | | | | | |
| Plasminogen | P00747 | LSSPAVITDK | 117 | b5 | 659.9 | 600.3 | 51 | 134827 | 34816 | 25.8 | | | | | | |
| Plasminogen | P00747 | LSSPAVITDK | 117 | y5 | 659.9 | 719.4 | 51 | 40441 | 13040 | 32.2 | | | | | | |
| Plasminogen | P00747 | QLGAGSIEECAAK | 113 | y3 | 538.6 | 429.3 | 45 | 146502 | 13270 | 9.1 | 1.06 | 0.05 | 4.7 | 1.06 | 0.04 | 4.0 |
| Plasminogen | P00747 | QLGAGSIEECAAK | 113 | b3 | 538.6 | 439.3 | 45 | 163553 | 12081 | 7.4 | 1.08 | 0.06 | 5.7 | | | |
| Plasminogen | P00747 | QLGAGSIEECAAK | 113 | b4 | 538.6 | 510.3 | 45 | 109829 | 9021 | 8.2 | 1.00 | 0.08 | 7.8 | | | |
| Plasminogen | P00747 | QLGAGSIEECAAK | 113 | b5 | 538.6 | 567.3 | 45 | 83079 | 7451 | 9.0 | 1.10 | 0.10 | 9.3 | | | |
| Plasminogen | P00747 | QLGAGSIEECAAK | 117 | y3 | 541.3 | 433.3 | 45 | 138228 | 14715 | 10.6 | | | | | | |
| Plasminogen | P00747 | QLGAGSIEECAAK | 117 | b3 | 541.3 | 443.3 | 45 | 151508 | 12502 | 8.3 | | | | | | |
| Plasminogen | P00747 | QLGAGSIEECAAK | 117 | b4 | 541.3 | 514.3 | 45 | 109982 | 8729 | 7.9 | | | | | | |
| Plasminogen | P00747 | QLGAGSIEECAAK | 117 | b5 | 541.3 | 571.3 | 45 | 75858 | 5959 | 7.9 | | | | | | |
| Plasminogen | P00747 | WELCDIPR | 113 | y3 | 614.8 | 385.3 | 49 | 625224 | 88625 | 14.2 | 0.92 | 0.04 | 4.2 | 1.07 | 0.11 | 10.3 |
| Plasminogen | P00747 | WELCDIPR | 113 | y4 | 614.8 | 500.3 | 49 | 73477 | 6100 | 8.3 | 1.09 | 0.10 | 9.3 | | | |
| Plasminogen | P00747 | WELCDIPR | 113 | y6 | 614.8 | 773.4 | 49 | 75889 | 11897 | 15.7 | 1.09 | 0.10 | 8.9 | | | |
| Plasminogen | P00747 | WELCDIPR | 113 | b3 | 614.8 | 569.3 | 49 | 113747 | 15430 | 13.6 | 1.18 | 0.09 | 7.3 | | | |
| Plasminogen | P00747 | WELCDIPR | 117 | y3 | 616.8 | 385.3 | 49 | 683201 | 98595 | 14.4 | | | | | | |
| Plasminogen | P00747 | WELCDIPR | 117 | y4 | 616.8 | 500.3 | 49 | 68268 | 9766 | 14.3 | | | | | | |
| Plasminogen | P00747 | WELCDIPR | 117 | y6 | 616.8 | 773.4 | 49 | 70083 | 10702 | 15.3 | | | | | | |
| Plasminogen | P00747 | WELCDIPR | 117 | b3 | 616.8 | 573.3 | 49 | 97087 | 15379 | 15.8 | | | | | | |
| S para/aryl 1 | P27169 | EVQPVELPNCNLVK | 113 | b3 | 640.4 | 497.3 | 50 | 1300083 | 353156 | 27.2 | 0.96 | 0.01 | 1.1 | 0.90 | 0.07 | 7.6 |
| Serum para/aryl 1 | P27169 | EVQPVELPNCNLVK | 113 | y3 | 640.4 | 499.4 | 50 | 127420 | 33943 | 26.6 | 0.95 | 0.07 | 7.2 | | | |
| Serum para/aryl 1 | P27169 | EVQPVELPNCNLVK | 113 | b4 | 640.4 | 594.3 | 50 | 65299 | 20080 | 30.8 | 0.84 | 0.09 | 10.3 | | | |
| Serum para/aryl 1 | P27169 | EVQPVELPNCNLVK | 113 | b6 | 640.4 | 822.4 | 50 | 38128 | 8058 | 21.1 | 0.84 | 0.06 | 7.0 | | | |
| Serum para/aryl 1 | P27169 | EVQPVELPNCNLVK | 117 | b3 | 643.0 | 501.3 | 50 | 1350601 | 372888 | 27.6 | | | | | | |
| Serum para/aryl 1 | P27169 | EVQPVELPNCNLVK | 117 | y3 | 643.0 | 503.4 | 50 | 133236 | 33800 | 25.4 | | | | | | |
| Serum para/aryl 1 | P27169 | EVQPVELPNCNLVK | 117 | b4 | 643.0 | 598.3 | 50 | 77018 | 20968 | 27.2 | | | | | | |
| Serum para/aryl 1 | P27169 | EVQPVELPNCNLVK | 117 | b6 | 643.0 | 826.4 | 50 | 45908 | 10419 | 22.7 | | | | | | |
| Serum para/aryl 1 | P27169 | STVELFK | 113 | y3 | 552.3 | 547.4 | 46 | 77146 | 20021 | 26.0 | 0.89 | 0.11 | 12.8 | 0.91 | 0.06 | 7.0 |
| Serum para/aryl 1 | P27169 | STVELFK | 113 | b3 | 552.3 | 428.3 | 46 | 54541 | 14745 | 27.0 | 0.86 | 0.10 | 11.9 | | | |
| Serum para/aryl 1 | P27169 | STVELFK | 113 | b4 | 552.3 | 557.3 | 46 | 67925 | 18846 | 27.7 | 0.89 | 0.07 | 7.4 | | | |
| Serum para/aryl 1 | P27169 | STVELFK | 113 | y5 | 552.3 | 775.5 | 46 | 7962 | 2709 | 34.0 | 1.00 | 0.24 | 24.4 | | | |
| Serum para/aryl 1 | P27169 | STVELFK | 117 | y3 | 556.3 | 551.4 | 46 | 86351 | 18324 | 21.2 | | | | | | |
| Serum para/aryl 1 | P27169 | STVELFK | 117 | b3 | 556.3 | 432.3 | 46 | 63753 | 16360 | 25.7 | | | | | | |
| Serum para/aryl 1 | P27169 | STVELFK | 117 | b4 | 556.3 | 561.3 | 46 | 76147 | 18910 | 24.8 | | | | | | |
| Serum para/aryl 1 | P27169 | STVELFK | 117 | y5 | 556.3 | 779.5 | 46 | 8201 | 2849 | 34.7 | | | | | | |
| Pls. rtl-bind. prot. | P02753 | DPNGLPPEAQK | 113 | b4 | 482.6 | 524.3 | 42 | 23263 | 7451 | 32.0 | 1.12 | 0.25 | 22.1 | 1.05 | 0.06 | 5.6 |
| Pls. rtl-bind. prot. | P02753 | DPNGLPPEAQK | 113 | y5 | 723.4 | 712.4 | 54 | 41601 | 12462 | 30.0 | 1.00 | 0.11 | 10.9 | | | |
| Pls. rtl-bind. prot. | P02753 | DPNGLPPEAQK | 113 | b4 | 723.4 | 524.3 | 54 | 46840 | 14369 | 30.7 | 1.01 | 0.10 | 10.1 | | | |
| Pls. rtl-bind. prot. | P02753 | DPNGLPPEAQK | 113 | b3 | 723.4 | 467.2 | 54 | 22671 | 4965 | 21.9 | 1.07 | 0.21 | 20.0 | | | |
| Pls. rtl-bind. prot. | P02753 | DPNGLPPEAQK | 117 | b4 | 485.3 | 528.3 | 42 | 19787 | 9947 | 50.3 | | | | | | |
| Pls. rtl-bind. prot. | P02753 | DPNGLPPEAQK | 117 | y5 | 727.4 | 716.4 | 54 | 42283 | 13701 | 32.4 | | | | | | |
| Pls. rtl-bind. prot. | P02753 | DPNGLPPEAQK | 117 | b4 | 727.4 | 528.3 | 54 | 46256 | 13777 | 29.8 | | | | | | |
| Pls. rtl-bind. prot. | P02753 | DPNGLPPEAQK | 117 | b3 | 727.4 | 471.2 | 54 | 22222 | 6968 | 31.4 | | | | | | |
| Pls. rtl-bind. prot. | P02753 | LLNLDGTCADSYSFVFSR | 113 | b3 | 735.7 | 481.3 | 55 | 1392139 | 142108 | 10.2 | 1.20 | 0.05 | 4.0 | 1.26 | 0.09 | 6.9 |
| Pls. rtl-bind. prot. | P02753 | LLNLDGTCADSYSFVFSR | 113 | y8 | 1103.0 | 992.5 | 73 | 194126 | 23181 | 11.9 | 1.18 | 0.12 | 9.8 | | | |
| Pls. rtl-bind. prot. | P02753 | LLNLDGTCADSYSFVFSR | 113 | b5 | 1103.0 | 709.4 | 73 | 142240 | 15400 | 10.8 | 1.37 | 0.12 | 8.4 | | | |
| Pls. rtl-bind. prot. | P02753 | LLNLDGTCADSYSFVFSR | 113 | b4 | 1103.0 | 594.4 | 73 | 99928 | 13611 | 13.6 | 1.27 | 0.14 | 11.1 | | | |
| Pls. rtl-bind. prot. | P02753 | LLNLDGTCADSYSFVFSR | 117 | b3 | 737.0 | 485.3 | 55 | 1164168 | 111247 | 9.6 | | | | | | |
| Pls. rtl-bind. prot. | P02753 | LLNLDGTCADSYSFVFSR | 117 | y8 | 1105.0 | 992.5 | 73 | 165297 | 25022 | 15.1 | | | | | | |
| Pls. rtl-bind. prot. | P02753 | LLNLDGTCADSYSFVFSR | 117 | b5 | 1105.0 | 713.4 | 73 | 104185 | 11943 | 11.5 | | | | | | |
| Pls. rtl-bind. prot. | P02753 | LLNLDGTCADSYSFVFSR | 117 | b4 | 1105.0 | 598.4 | 73 | 78857 | 11239 | 14.3 | | | | | | |
| Pls. rtl-bind. prot. | P02753 | YWGVASFLQK | 113 | b3 | 739.9 | 547.3 | 55 | 124933 | 12573 | 10.1 | 1.15 | 0.06 | 5.3 | 1.09 | 0.07 | 6.2 |
| Pls. rtl-bind. prot. | P02753 | YWGVASFLQK | 113 | y4 | 739.9 | 528.4 | 55 | 85730 | 12004 | 14.0 | 1.11 | 0.15 | 13.4 | | | |
| Pls. rtl-bind. prot. | P02753 | YWGVASFLQK | 113 | y5 | 739.9 | 762.5 | 55 | 35630 | 4191 | 11.8 | 0.99 | 0.16 | 16.4 | | | |
| Pls. rtl-bind. prot. | P02753 | YWGVASFLQK | 113 | y6 | 739.9 | 833.5 | 55 | 26338 | 2680 | 10.2 | 1.09 | 0.13 | 12.0 | | | |
| Pls. rtl-bind. prot. | P02753 | YWGVASFLQK | 117 | b3 | 743.9 | 551.3 | 55 | 108484 | 12415 | 11.4 | | | | | | |
| Pls. rtl-bind. prot. | P02753 | YWGVASFLQK | 117 | y4 | 743.9 | 532.4 | 55 | 77486 | 7517 | 9.7 | | | | | | |
| Pls. rtl-bind. prot. | P02753 | YWGVASFLQK | 117 | y5 | 743.9 | 766.5 | 55 | 36613 | 6674 | 18.2 | | | | | | |
| Pls. rtl-bind. prot. | P02753 | YWGVASFLQK | 117 | y6 | 743.9 | 837.5 | 55 | 24373 | 3476 | 14.3 | | | | | | |
| Prothrombin | P00734 | ETAASLLQAGYK | 113 | b3 | 511.3 | 442.2 | 44 | 212418 | 24156 | 11.4 | 0.97 | 0.07 | 6.8 | 0.97 | 0.09 | 9.3 |
| Prothrombin | P00734 | ETAASLLQAGYK | 113 | b4 | 511.3 | 513.3 | 44 | 124047 | 17054 | 13.7 | 1.03 | 0.08 | 8.2 | | | |
| Prothrombin | P00734 | ETAASLLQAGYK | 113 | y3 | 511.3 | 507.3 | 44 | 60813 | 10388 | 17.1 | 1.05 | 0.09 | 8.6 | | | |
| Prothrombin | P00734 | ETAASLLQAGYK | 113 | y4 | 511.3 | 578.3 | 44 | 20455 | 4837 | 23.6 | 0.85 | 0.14 | 17.1 | | | |
| Prothrombin | P00734 | ETAASLLQAGYK | 117 | b3 | 514.0 | 446.2 | 44 | 218946 | 22060 | 10.1 | | | | | | |
| Prothrombin | P00734 | ETAASLLQAGYK | 117 | b4 | 514.0 | 517.3 | 44 | 120367 | 11755 | 9.8 | | | | | | |
| Prothrombin | P00734 | ETAASLLQAGYK | 117 | y3 | 514.0 | 511.3 | 44 | 57899 | 6336 | 10.9 | | | | | | |
| Prothrombin | P00734 | ETAASLLQAGYK | 117 | y4 | 514.0 | 582.3 | 44 | 24157 | 3224 | 13.3 | | | | | | |
| Prothrombin | P00734 | ETWTANVGK | 113 | y3 | 643.4 | 443.3 | 50 | 137455 | 15801 | 11.5 | 1.14 | 0.09 | 8.0 | 1.11 | 0.04 | 3.7 |
| Prothrombin | P00734 | ETWTANVGK | 113 | y4 | 643.4 | 557.3 | 50 | 67494 | 9154 | 13.6 | 1.14 | 0.07 | 6.5 | | | |
| Prothrombin | P00734 | ETWTANVGK | 113 | y5 | 643.4 | 628.4 | 50 | 46547 | 5652 | 12.1 | 1.05 | 0.15 | 14.4 | | | |
| Prothrombin | P00734 | ETWTANVGK | 113 | b3 | 643.4 | 557.3 | 50 | 76540 | 9599 | 12.5 | 1.13 | 0.04 | 3.9 | | | |
| Prothrombin | P00734 | ETWTANVGK | 117 | y3 | 647.4 | 447.3 | 50 | 121570 | 18522 | 15.2 | | | | | | |
| Prothrombin | P00734 | ETWTANVGK | 117 | y4 | 647.4 | 561.3 | 50 | 59266 | 6660 | 11.2 | | | | | | |
| Prothrombin | P00734 | ETWTANVGK | 117 | y5 | 647.4 | 632.4 | 50 | 44984 | 7816 | 17.4 | | | | | | |
| Prothrombin | P00734 | ETWTANVGK | 117 | b3 | 647.4 | 561.3 | 50 | 68004 | 7988 | 11.7 | | | | | | |
| Transthyretin | P02766 | AADDTWEPFASGK | 113 | b4 | 558.9 | 513.2 | 46 | 370677 | 35347 | 9.5 | 0.96 | 0.06 | 6.3 | 1.04 | 0.14 | 13.7 |
| Transthyretin | P02766 | AADDTWEPFASGK | 113 | y4 | 558.9 | 502.3 | 46 | 172538 | 18590 | 10.8 | 0.89 | 0.05 | 6.0 | | | |
| Transthyretin | P02766 | AADDTWEPFASGK | 113 | b4 | 837.9 | 513.2 | 60 | 212363 | 43583 | 20.5 | 1.12 | 0.03 | 3.1 | | | |
| Transthyretin | P02766 | AADDTWEPFASGK | 113 | y4 | 837.9 | 502.3 | 60 | 102139 | 22640 | 22.2 | 1.20 | 0.05 | 4.0 | | | |
| Transthyretin | P02766 | AADDTWEPFASGK | 117 | b4 | 561.6 | 517.2 | 46 | 388161 | 33829 | 8.7 | | | | | | |
| Transthyretin | P02766 | AADDTWEPFASGK | 117 | y4 | 561.6 | 506.3 | 46 | 193300 | 13904 | 7.2 | | | | | | |
| Transthyretin | P02766 | AADDTWEPFASGK | 117 | b4 | 841.9 | 517.2 | 60 | 188816 | 38529 | 20.4 | | | | | | |
| Transthyretin | P02766 | AADDTWEPFASGK | 117 | y4 | 841.9 | 506.3 | 60 | 85374 | 18959 | 22.2 | | | | | | |
| Transthyretin | P02766 | GSPAINVAVHVFR | 113 | b4 | 503.0 | 453.2 | 43 | 1810438 | 128371 | 7.1 | 0.96 | 0.02 | 1.9 | 0.94 | 0.02 | 2.5 |
| Transthyretin | P02766 | GSPAINVAVHVFR | 113 | y3 | 503.0 | 421.3 | 43 | 279811 | 24290 | 8.7 | 0.95 | 0.04 | 3.8 | | | |
| Transthyretin | P02766 | GSPAINVAVHVFR | 113 | y4 | 503.0 | 558.3 | 43 | 96400 | 10638 | 11.0 | 0.95 | 0.07 | 7.9 | | | |
| Transthyretin | P02766 | GSPAINVAVHVFR | 113 | b6 | 503.0 | 680.4 | 43 | 126661 | 10337 | 8.2 | 0.91 | 0.04 | 4.1 | | | |
| Transthyretin | P02766 | GSPAINVAVHVFR | 117 | b4 | 504.3 | 457.3 | 43 | 1888411 | 132060 | 7.0 | | | | | | |
| Transthyretin | P02766 | GSPAINVAVHVFR | 117 | y3 | 504.3 | 421.3 | 43 | 295474 | 23054 | 7.8 | | | | | | |
| Transthyretin | P02766 | GSPAINVAVHVFR | 117 | y4 | 504.3 | 558.3 | 43 | 102213 | 11224 | 11.0 | | | | | | |
| Transthyretin | P02766 | GSPAINVAVHVFR | 117 | b6 | 504.3 | 684.4 | 43 | 140078 | 12826 | 9.2 | | | | | | |
| Transthyretin | P02766 | YTIAALLSPYSYSTTAVVTNPKE | 113 | b5 | 923.8 | 660.4 | 64 | 444209 | 43609 | 9.8 | 1.17 | 0.03 | 2.4 | 1.20 | 0.12 | 9.8 |
| Transthyretin | P02766 | YTIAALLSPYSYSTTAVVTNPKE | 113 | b4 | 923.8 | 589.3 | 64 | 272846 | 23652 | 8.7 | 1.04 | 0.03 | 2.6 | | | |
| Transthyretin | P02766 | YTIAALLSPYSYSTTAVVTNPKE | 113 | b6 | 923.8 | 773.5 | 64 | 181673 | 20687 | 11.4 | 1.28 | 0.05 | 3.5 | | | |
| Transthyretin | P02766 | YTIAALLSPYSYSTTAVVTNPKE | 113 | y3 | 923.8 | 513.3 | 64 | 495637 | 50650 | 10.2 | 1.30 | 0.03 | 2.1 | | | |
| Transthyretin | P02766 | YTIAALLSPYSYSTTAVVTNPKE | 117 | b5 | 926.5 | 664.4 | 64 | 378446 | 34183 | 9.0 | | | | | | |
| Transthyretin | P02766 | YTIAALLSPYSYSTTAVVTNPKE | 117 | b4 | 926.5 | 593.3 | 64 | 261413 | 24540 | 9.4 | | | | | | |
| Transthyretin | P02766 | YTIAALLSPYSYSTTAVVTNPKE | 117 | b6 | 926.5 | 777.5 | 64 | 141976 | 17515 | 12.3 | | | | | | |
| Transthyretin | P02766 | YTIAALLSPYSYSTTAVVTNPKE | 117 | y3 | 926.5 | 517.3 | 64 | 381598 | 38387 | 10.1 | | | | | | |
| Vt D-bind. prot. | P02797 | HQPQEFPTYVEPTNDEICEAFR | 113 | b6 | 949.8 | 907.4 | 65 | 226018 | 40306 | 17.8 | 1.21 | 0.05 | 4.2 | 1.01 | 0.19 | 18.7 |
| Vt D-bind. prot. | P02797 | HQPQEFPTYVEPTNDEICEAFR | 113 | b5 | 949.8 | 760.4 | 65 | 612297 | 101356 | 16.6 | 1.11 | 0.02 | 1.9 | | | |
| Vt D-bind. prot. | P02797 | HQPQEFPTYVEPTNDEICEAFR | 113 | y4 | 949.8 | 522.3 | 65 | 181525 | 32064 | 17.7 | 0.78 | 0.04 | 4.5 | | | |
| Vt D-bind. prot. | P02797 | HQPQEFPTYVEPTNDEICEAFR | 113 | y6 | 949.8 | 795.4 | 65 | 218332 | 38574 | 17.7 | 0.94 | 0.03 | 3.0 | | | |
| Vt D-bind. prot. | P02797 | HQPQEFPTYVEPTNDEICEAFR | 117 | b6 | 951.1 | 911.4 | 66 | 186358 | 34171 | 18.3 | | | | | | |
| Vt D-bind. prot. | P02797 | HQPQEFPTYVEPTNDEICEAFR | 117 | b5 | 951.1 | 764.4 | 66 | 551378 | 90055 | 16.3 | | | | | | |
| Vt D-bind. prot. | P02797 | HQPQEFPTYVEPTNDEICEAFR | 117 | y4 | 951.1 | 522.3 | 66 | 231096 | 35246 | 15.3 | | | | | | |
| Vt D-bind. prot. | P02797 | HQPQEFPTYVEPTNDEICEAFR | 117 | y6 | 951.1 | 795.4 | 66 | 231826 | 37548 | 16.2 | | | | | | |
| Vt D-bind. prot. | P02821 | LCDNLSTK | 113 | y3 | 410.9 | 475.3 | 39 | 46543 | 8772 | 18.8 | 1.12 | 0.11 | 9.4 | 1.10 | 0.06 | 5.7 |
| Vt D-bind. prot. | P02820 | LCDNLSTK | 113 | b3 | 615.8 | 529.2 | 49 | 379776 | 37521 | 9.9 | 1.11 | 0.04 | 3.7 | | | |
| Vt D-bind. prot. | P02820 | LCDNLSTK | 113 | b4 | 615.8 | 643.3 | 49 | 292142 | 33812 | 11.6 | 1.15 | 0.05 | 4.2 | | | |
| Vt D-bind. prot. | P02820 | LCDNLSTK | 113 | y5 | 615.8 | 702.4 | 49 | 78600 | 8549 | 10.9 | 1.01 | 0.10 | 9.8 | | | |
| Vt D-bind. prot. | P02821 | LCDNLSTK | 117 | y4 | 413.6 | 479.3 | 39 | 42055 | 9994 | 23.8 | | | | | | |
| Vt D-bind. prot. | P02820 | LCDNLSTK | 117 | b3 | 619.8 | 533.3 | 49 | 343766 | 37818 | 11.0 | | | | | | |
| Vt D-bind. prot. | P02820 | LCDNLSTK | 117 | b4 | 619.8 | 647.3 | 49 | 254043 | 26002 | 10.2 | | | | | | |
| Vt D-bind. prot. | P02820 | LCDNLSTK | 117 | y5 | 619.8 | 706.4 | 49 | 78530 | 9847 | 12.5 | | | | | | |
| Vt D-bind. prot. | P02790 | SCESNSPFPVHPGTAECCTK | 113 | b5 | 849.0 | 718.3 | 60 | 125284 | 35999 | 28.7 | 0.92 | 0.08 | 8.9 | 0.90 | 0.03 | 2.9 |
| Vt D-bind. prot. | P02791 | SCESNSPFPVHPGTAECCTK | 113 | b3 | 849.0 | 517.2 | 60 | 121924 | 32659 | 26.8 | 0.92 | 0.05 | 5.4 | | | |
| Vt D-bind. prot. | P02791 | SCESNSPFPVHPGTAECCTK | 113 | y4 | 849.0 | 708.3 | 60 | 72842 | 19476 | 26.7 | 0.87 | 0.08 | 9.8 | | | |
| Vt D-bind. prot. | P02791 | SCESNSPFPVHPGTAECCTK | 113 | b6 | 849.0 | 805.3 | 60 | 119812 | 35833 | 29.9 | 0.91 | 0.06 | 6.5 | | | |
| Vt D-bind. prot. | P02790 | SCESNSPFPVHPGTAECCTK | 117 | b5 | 851.7 | 722.3 | 61 | 135605 | 38565 | 28.4 | | | | | | |
| Vt D-bind. prot. | P02791 | SCESNSPFPVHPGTAECCTK | 117 | b3 | 851.7 | 521.2 | 61 | 133248 | 36642 | 27.5 | | | | | | |
| Vt D-bind. prot. | P02791 | SCESNSPFPVHPGTAECCTK | 117 | y4 | 851.7 | 712.3 | 61 | 85573 | 25834 | 30.2 | | | | | | |
| Vt D-bind. prot. | P02791 | SCESNSPFPVHPGTAECCTK | 117 | b6 | 851.7 | 809.3 | 61 | 132475 | 41147 | 31.1 | | | | | | |
| Vt D-bind. prot. | P02845 | SLGECCDVEDSTTCFNAK | 113 | b4 | 791.6 | 527.3 | 58 | 1013334 | 277605 | 27.4 | 1.00 | 0.03 | 2.9 | 1.14 | 0.11 | 9.5 |
| Vt D-bind. prot. | P02844 | SLGECCDVEDSTTCFNAK | 113 | b4 | 1187.0 | 527.3 | 77 | 320641 | 54263 | 16.9 | 1.10 | 0.03 | 3.1 | | | |
| Vt D-bind. prot. | P02846 | SLGECCDVEDSTTCFNAK | 113 | b7 | 1187.0 | 962.4 | 77 | 233640 | 38071 | 16.3 | 1.22 | 0.03 | 2.6 | | | |
| Vt D-bind. prot. | P02846 | SLGECCDVEDSTTCFNAK | 113 | y4 | 1187.0 | 619.4 | 77 | 165089 | 21002 | 12.7 | 1.23 | 0.09 | 7.0 | | | |
| Vt D-bind. prot. | P02845 | SLGECCDVEDSTTCFNAK | 117 | b4 | 794.3 | 531.3 | 58 | 1013221 | 278741 | 27.5 | | | | | | |
| Vt D-bind. prot. | P02844 | SLGECCDVEDSTTCFNAK | 117 | b4 | 1191.0 | 531.3 | 78 | 291994 | 51705 | 17.7 | | | | | | |
| Vt D-bind. prot. | P02846 | SLGECCDVEDSTTCFNAK | 117 | b7 | 1191.0 | 966.4 | 78 | 192355 | 31966 | 16.6 | | | | | | |
| Vt D-bind. prot. | P02846 | SLGECCDVEDSTTCFNAK | 117 | y4 | 1191.0 | 623.4 | 78 | 135125 | 21835 | 16.2 | | | | | | |
| Vt D-bind. prot. | P02805 | VCSQYAAYGEK | 113 | y3 | 519.2 | 473.3 | 44 | 422127 | 78240 | 18.5 | 1.44 | 0.04 | 2.9 | 1.40 | 0.10 | 6.9 |
| Vt D-bind. prot. | P02805 | VCSQYAAYGEK | 113 | b4 | 519.2 | 615.3 | 44 | 213702 | 46117 | 21.6 | 1.28 | 0.06 | 4.8 | | | |
| Vt D-bind. prot. | P02804 | VCSQYAAYGEK | 113 | y3 | 778.4 | 473.3 | 57 | 370445 | 119880 | 32.4 | 1.38 | 0.06 | 4.7 | | | |
| Vt D-bind. prot. | P02806 | VCSQYAAYGEK | 113 | y4 | 778.4 | 636.3 | 57 | 157359 | 63293 | 40.2 | 1.51 | 0.11 | 7.4 | | | |
| Vt D-bind. prot. | P02805 | VCSQYAAYGEK | 117 | y4 | 521.9 | 477.3 | 44 | 294373 | 57661 | 19.6 | | | | | | |
| Vt D-bind. prot. | P02805 | VCSQYAAYGEK | 117 | b4 | 521.9 | 619.4 | 44 | 167617 | 38472 | 23.0 | | | | | | |
| Vt D-bind. prot. | P02804 | VCSQYAAYGEK | 117 | y3 | 782.4 | 477.3 | 57 | 269492 | 90025 | 33.4 | | | | | | |
| Vt D-bind. prot. | P02806 | VCSQYAAYGEK | 117 | y4 | 782.4 | 640.3 | 57 | 105768 | 46013 | 43.5 | | | | | | |
| Vitronectin | P04004 | DVWGIEGPIDAAFTR | 113 | y5 | 596.3 | 565.3 | 48 | 707691 | 49690 | 7.0 | 0.99 | 0.02 | 1.7 | 0.99 | 0.04 | 3.7 |
| Vitronectin | P04004 | DVWGIEGPIDAAFTR | 113 | y6 | 596.3 | 680.3 | 48 | 361266 | 28305 | 7.8 | 0.99 | 0.01 | 1.3 | | | |
| Vitronectin | P04004 | DVWGIEGPIDAAFTR | 113 | y5 | 894.0 | 565.3 | 63 | 743207 | 33688 | 4.5 | 0.96 | 0.01 | 1.5 | | | |
| Vitronectin | P04004 | DVWGIEGPIDAAFTR | 113 | b4 | 894.0 | 598.3 | 63 | 464668 | 21954 | 4.7 | 1.04 | 0.02 | 2.4 | | | |
| Vitronectin | P04004 | DVWGIEGPIDAAFTR | 117 | y5 | 597.6 | 565.3 | 48 | 716322 | 54743 | 7.6 | | | | | | |
| Vitronectin | P04004 | DVWGIEGPIDAAFTR | 117 | y6 | 597.6 | 680.3 | 48 | 366253 | 31001 | 8.5 | | | | | | |
| Vitronectin | P04004 | DVWGIEGPIDAAFTR | 117 | y5 | 896.0 | 565.3 | 63 | 777369 | 28058 | 3.6 | | | | | | |
| Vitronectin | P04004 | DVWGIEGPIDAAFTR | 117 | b4 | 896.0 | 602.3 | 63 | 445034 | 22161 | 5.0 | | | | | | |
| Vitronectin | P04051 | DWHGVPGQVDAAMAGR | 113 | b4 | 603.0 | 636.3 | 48 | 181802 | 11648 | 6.4 | 0.97 | 0.03 | 3.2 | 0.95 | 0.03 | 3.5 |
| Vitronectin | P04050 | DWHGVPGQVDAAMAGR | 113 | y5 | 603.0 | 505.3 | 48 | 193524 | 39144 | 20.2 | 0.90 | 0.06 | 6.9 | | | |
| Vitronectin | P04051 | DWHGVPGQVDAAMAGR | 113 | y7 | 603.0 | 691.3 | 48 | 155633 | 11896 | 7.6 | 0.95 | 0.04 | 3.8 | | | |
| Vitronectin | P04048 | DWHGVPGQVDAAMAGR | 113 | y8 | 603.0 | 790.4 | 48 | 49941 | 5357 | 10.7 | 0.96 | 0.10 | 10.5 | | | |
| Vitronectin | P04051 | DWHGVPGQVDAAMAGR | 117 | b4 | 604.3 | 640.3 | 48 | 186685 | 13103 | 7.0 | | | | | | |
| Vitronectin | P04050 | DWHGVPGQVDAAMAGR | 117 | y5 | 604.3 | 505.3 | 48 | 217494 | 59355 | 27.3 | | | | | | |
| Vitronectin | P04051 | DWHGVPGQVDAAMAGR | 117 | y7 | 604.3 | 691.3 | 48 | 163225 | 10636 | 6.5 | | | | | | |
| Vitronectin | P04048 | DWHGVPGQVDAAMAGR | 117 | y8 | 604.3 | 790.4 | 48 | 51924 | 4184 | 8.1 | | | | | | |
| Vitronectin | P04004 | GQYCYELDEK | 113 | b3 | 528.9 | 489.2 | 44 | 195667 | 34368 | 17.6 | 1.11 | 0.04 | 3.4 | 1.02 | 0.09 | 9.1 |
| Vitronectin | P04004 | GQYCYELDEK | 113 | b3 | 792.9 | 489.2 | 58 | 139035 | 45148 | 32.5 | 1.06 | 0.08 | 7.4 | | | |
| Vitronectin | P04004 | GQYCYELDEK | 113 | b4 | 792.9 | 649.3 | 58 | 48008 | 16641 | 34.7 | 0.89 | 0.07 | 7.9 | | | |
| Vitronectin | P04004 | GQYCYELDEK | 113 | y3 | 792.9 | 531.3 | 58 | 90965 | 31133 | 34.2 | 1.01 | 0.08 | 7.9 | | | |
| Vitronectin | P04004 | GQYCYELDEK | 117 | b3 | 531.6 | 493.3 | 45 | 176233 | 29537 | 16.8 | | | | | | |
| Vitronectin | P04004 | GQYCYELDEK | 117 | b3 | 796.9 | 493.3 | 58 | 132710 | 43871 | 33.1 | | | | | | |
| Vitronectin | P04004 | GQYCYELDEK | 117 | b4 | 796.9 | 653.3 | 58 | 53382 | 17195 | 32.2 | | | | | | |
| Vitronectin | P04004 | GQYCYELDEK | 117 | y3 | 796.9 | 535.3 | 58 | 91232 | 33673 | 36.9 | | | | | | |
| Vitronectin | P04067 | SIAQYWLGCPAPGHL | 113 | b4 | 604.0 | 540.3 | 48 | 377689 | 13177 | 3.5 | 0.69 | 0.04 | 6.0 | 1.35 | 0.62 | 45.7 |
| Vitronectin | P04064 | SIAQYWLGCPAPGHL | 113 | y4 | 905.5 | 423.2 | 63 | 1065872 | 47090 | 4.4 | 2.18 | 0.10 | 4.7 | | | |
| Vitronectin | P04066 | SIAQYWLGCPAPGHL | 113 | b4 | 905.5 | 540.3 | 63 | 446731 | 29520 | 6.6 | 1.32 | 0.03 | 2.0 | | | |
| Vitronectin | P04064 | SIAQYWLGCPAPGHL | 113 | b5 | 905.5 | 703.4 | 63 | 237254 | 14702 | 6.2 | 1.21 | 0.04 | 3.6 | | | |
| Vitronectin | P04067 | SIAQYWLGCPAPGHL | 117 | b4 | 605.3 | 544.3 | 48 | 546123 | 31683 | 5.8 | | | | | | |
| Vitronectin | P04064 | SIAQYWLGCPAPGHL | 117 | y4 | 907.5 | 423.2 | 63 | 489664 | 40978 | 8.4 | | | | | | |
| Vitronectin | P04066 | SIAQYWLGCPAPGHL | 117 | b4 | 907.5 | 544.3 | 63 | 337458 | 24150 | 7.2 | | | | | | |
| Vitronectin | P04064 | SIAQYWLGCPAPGHL | 117 | b5 | 907.5 | 707.4 | 63 | 196789 | 14606 | 7.4 | | | | | | |

TABLE 5

| | |
|---|---|
| Peptide Sequence | SEQ. ID. No. |

| | |
|---|---|
| CIGETIGR | 1 |
| CIGEIPAK | 2 |
| CIGEELSK | 3 |
| YCFGEGLAR | 4 |
| FCLGESLAK | 5 |
| ICLGESIAR | 6 |
| ICAGEGLAR | 7 |
| VCAGEGLAR | 8 |
| ICVGESLAR | 9 |
| SCLGEALAR | 10 |
| SCLGEPLAR | 11 |
| VCVGEGLAR | 12 |
| LCLGEPLAR | 13 |
| ACLGEQLAK | 14 |
| NCLGMR | 15 |
| NCIGK | 16 |
| YIDLLPTSLPHAVTCDIK | 17 |
| ICVGEGLAR | 18 |
| ACLGEPLAR | 19 |
| CIGEVLAK | 20 |
| GFCMFDMECHK | 21 |
| ICLGEGIAR | 22 |
| LCQNEGCK | 23 |
| GCPSLSELWR | 24 |
| EECALEIIK | 25 |
| GCPSLAEHWK | 26 |
| VFANPEDCAFGK | 27 |
| SDVVYTDWK | 28 |
| YVGGQEHFAHLLILR | 29 |
| ATWSGAVLAGR | 30 |
| LLELTGPK | 31 |
| SGLSTGWTQLSK | 32 |
| AIGYLNTGYQR | 33 |
| NEDSLVFVQTDK | 34 |
| SSGSLLNNAIK | 35 |
| ADLSGITGAR | 36 |
| AVLDVFEEGTEASAATAVK | 37 |
| EIGELYLPK | 38 |
| ITLLSALVETR | 39 |
| LYGSEAFATDFQDSAAAK | 40 |
| AFIQLWAFDAVK | 41 |
| TVAACNLPIVR | 42 |
| ALQDQLVLVAAK | 43 |
| QPFVQGLALYTPVVLPR | 44 |
| SLDFTELDVAAEK | 45 |
| EVPLNTIIFMGR | 46 |
| LQPLDFK | 47 |
| TSDQIHFFFAK | 48 |
| DLATVYVDVLK | 49 |
| DYVSQFEGSALGK | 50 |
| LLDNWDSVTSTFSK | 51 |
| LSPLGEEMR | 52 |
| VQPYLDDFQK | 53 |
| AGTELVNFLSYFVELGTQPATQ | 54 |
| EQLTPLIK | 55 |
| SPELQAEAK | 56 |
| ALVQQMEQLR | 57 |
| ISASAEELR | 58 |
| LEPYADQLR | 59 |
| LGEVNTYAGDLQK | 60 |
| SELTQQLNALFQDK | 61 |
| TQVNTQAEQLR | 62 |
| NNALDFVTK | 63 |
| SVSLPSLDPASAK | 64 |
| YGMVAQVTQTLK | 65 |
| EFGNTLEDK | 66 |
| EWFSETFQK | 67 |
| DALSSVQESQVAQQAR | 68 |
| GWVTDGFSSLK | 69 |
| SEAEDASLLSFMQGYMK | 70 |
| LGPLVEQGR | 71 |
| LQAEAFQAR | 72 |
| ATFGCHDGYSLDGPEEIECTK | 73 |
| ATVVYQGER | 74 |
| VCPFAGILENGAVR | 75 |
| FQPTLLTLPR | 76 |
| LEDMEQALSPSVFK | 77 |
| LLDSLPSDTR | 78 |
| ALYLQYTDETFR | 79 |
| DIASGLIGPLIICK | 80 |
| EVGPTNADPVCLAK | 81 |
| GAYPLSIEPIGVR | 82 |
| DISEVVTPR | 83 |
| EAGIPEFYDYDVALIK | 84 |
| EELLPAQDIK | 85 |
| GDSGGPLIVHK | 86 |
| VSEADSSNADWVTK | 87 |
| YGLVTYATYPK | 88 |
| DGWSAQPTCIK | 89 |
| ECDTDGWTNDIPICEVVK | 90 |
| LSYTCEGGFR | 91 |
| SSNLIILEEHLK | 92 |
| WQSIPLCVEK | 93 |
| ASSIIDELFQDR | 94 |
| ELDESLQVAER | 95 |
| LFDSDPITVTVPVEVSR | 96 |
| EYVLPSFEVIVEPTEK | 97 |
| ILLQGTPVAQMTEDAVDAER | 98 |
| IPIEDGSGEVVLSR | 99 |
| SSLSVPYVIVPLK | 100 |
| TGLQEVEVK | 101 |
| GLEEELQFSLGSK | 102 |
| LGQYASPTAK | 103 |
| VLSLAQEQVGGSPEK | 104 |
| AEFQDALEK | 105 |
| LNMGITDLQGLR | 106 |
| VDFTLSSER | 107 |
| VGDTLNLNLR | 108 |
| VGDTLNLNLR | 109 |
| AIEDYINEFSVR | 110 |
| LSPIYNLVPVK | 111 |
| VVEESELAR | 112 |
| EHAVEGDCDFQLLK | 113 |
| FSVVYAK | 114 |
| HTLNQIDEVK | 115 |
| DLQFVEVTDVK | 116 |
| IYLYTLNDNAR | 117 |
| STTPDITGYR | 118 |
| WLPSSSPVTGYR | 119 |
| AGALNSNDAFVLK | 120 |
| AQPVQVAEGSEPDGFWEALGGK | 121 |
| TASDFITK | 122 |
| DYFMPCPGR | 123 |
| GECQAEGVLFFQGDR | 124 |
| GGYTLVSGYPK | 125 |
| LLQDEFPGIPSPLDAAVECHR | 126 |
| NFPSPVDAAFR | 127 |
| VDGALCMEK | 128 |
| YYCFQGNQFLR | 129 |
| IAIDLFK | 130 |
| NYNLVESLK | 131 |
| TLEAQLTPR | 132 |
| DGYLFQLLR | 133 |
| DSPVLIDFFEDTER | 134 |
| GGEGTGYFVDFSVR | 135 |
| ELAAQTIK | 136 |
| FAHYVVTSQVVNTANEAR | 137 |
| LDAQASFLPK | 138 |
| NDLISATK | 139 |
| SLAPTAAAK | 140 |
| SSALDMENFR | 141 |
| TEVNVLPGAK | 142 |
| VQFELHYQEVK | 143 |
| VVNNSPQPQNVVFDVQIPK | 144 |
| GSEMVVAGK | 145 |
| ILDDLSPR | 146 |
| NVVFVIDK | 147 |
| DIPTNSPELEETLTHTITK | 148 |
| ENFLFLTPDCK | 149 |
| TVGSDTFYSFK | 150 |
| YFIDFVAR | 151 |
| EAQLPVIENK | 152 |
| EPLDDYVNTQGASLFSVTK | 153 |
| LSSPAVITDK | 154 |
| QLGAGSIEECAAK | 155 |
| WELCDIPR | 156 |
| EVQPVELPNCNLVK | 157 |
| STVELFK | 158 |
| DPNGLPPEAQK | 159 |
| LLNLDGTCADSYSFVFSR | 160 |
| YWGVASFLQK | 161 |
| ETAASLLQAGYK | 162 |
| ETWTANVGK | 163 |
| AADDTWEPFASGK | 164 |
| GSPAINVAVHVFR | 165 |
| YTIAALLSPYSYSTTAVVTNPKE | 166 |
| HQPQEFPTYVEPTNDEICEAFR | 167 |
| LCDNLSTK | 168 |
| SCESNSPFPVHPGTAECCTK | 169 |
| SLGECCDVEDSTTCFNAK | 170 |
| VCSQYAAYGEK | 171 |
| DVWGIEGPIDAAFTR | 172 |
| DWHGVPGQVDAAMAGR | 173 |
| GQYCYELDEK | 174 |
| SIAQYWLGCPAPGHL | 175 |
| GAGTGGLGLAVEGPSEAK | 176 |
| LQAAGIQLHNVWAR | 177 |

While the teachings have been particularly shown and described with reference to specific illustrative embodiments, it should be understood that various changes in form and detail may be made without departing from the spirit and scope of the teachings. For example, any of the various disclosed labeling approaches, PDITM approaches, concentration curves, and mass analyzer systems and can be combined to provide a method for determining the absolute concentration of a protein, or multiple proteins, in a sample or multiple samples. Therefore, all embodiments that come within the scope and spirit of the teachings, and equivalents thereto are claimed. The descriptions and diagrams of the methods, systems, and assays of the present teachings should not be read as limited to the described order of elements unless stated to that effect.

What is claimed is:

1. A method for assessing the biological state of a sample, comprising the steps of:
providing a standard sample comprising a signature peptide for each protein of interest;
selecting a diagnostic daughter ion for each signature peptide;
labeling the one or more proteins of interest in two or more samples of interest with different chemical moieties for each sample, the two or more samples of interest thereby being differentially labeled;
labeling one or more standard samples with a chemical moiety;
combining, to produce a combined sample, at least a portion of the one or more labeled standard samples with at least a portion of two or more differentially labeled samples, the differentially labeled samples being labeled with a different chemical moiety than the one or more labeled standard samples combined therewith;
loading at least a portion of the combined sample on a chromatographic column;
subjecting at least a portion of the eluent from the chromatographic column to multiple reaction monitoring, the transmitted parent ion m/z range of each multiple reaction monitoring scan including a m/z value of one or more of the labeled signature peptides and the transmitted daughter ion m/z range of each multiple reaction monitoring scan including a m/z value one or more of the selected diagnostic daughter ions corresponding to the transmitted labeled signature peptide;
measuring the ion signal of one or more of the selected diagnostic daughter ions using said multiple reaction monitoring; and
determining the concentration of a protein of interest in one or more of the two or more samples of interest based at least on a comparison of the measured ion signal of a selected diagnostic daughter ion corresponding to the protein of interest from a sample of interest to the measured ion signal for the selected diagnostic daughter ion corresponding to the protein of interest from a labeled standard sample; and
assessing the biological state of the sample based at least on a comparison of the relative concentrations of two or more proteins in one or more of the two or more samples to the concentration of two or more corresponding proteins in one or more of the standard samples; wherein
the one or more proteins of interest are one or more of the proteins listed in column 1 of Table 4; and further wherein
the signature peptide comprises one or more of the peptides listed in column 3 of Table 4, which corresponds to a respective protein of interest listed in column 1 of Table 4.

2. The method of claim 1, wherein the step of labeling proteins of interest in one or more standard samples comprises labeling proteins of interest with an isotopically coded affinity tag, and wherein the step of labeling proteins of interest in different samples comprises labeling proteins of interest with an isotopically coded affinity tag.

3. The method of claim 1, wherein the step of labeling proteins of interest in one or more standard samples comprises labeling proteins of interest with an isobaric tag, and wherein the step of labeling proteins of interest in different samples comprises labeling proteins of interest with an isobaric tag.

4. The method of claim 1, wherein the step of labeling proteins of interest in one or more standard samples comprises labeling proteins of interest with a mass differential tag, and wherein the step of labeling proteins of interest in different samples comprises labeling proteins of interest with a mass differential tag.

5. The method of claim 1, wherein the one or more standard samples comprise a pooled reference sample.

6. The method of claim 1, further comprising the step of subjecting at least a portion of the combined sample to digestion to produce a digested combined sample prior to loading of at least a portion of the combined sample on a chromatographic column, and wherein the portion of the combined sample on a chromatographic column is all or a portion of the digested combined sample.

7. The method of claim 6, wherein the digestion comprises chemical digestion.

8. The method of claim 6, wherein the digestion comprises enzymatic digestion.

9. The method of claim 1, wherein one or more of the one or more of the standard samples are subjected to a digestion prior to being combined with the two or more labeled samples of interest to produce a combined sample.

10. The method of claim 9, wherein the digestion comprises chemical digestion.

11. The method of claim 9, wherein the digestion comprises enzymatic digestion.

12. The method of claim 1, wherein the step of determining the concentration of a protein of interest comprises determining the absolute concentration of the protein of interest.

13. The method of claim 1, the step of determining the concentration of a protein of interest comprises determining the relative concentration of the protein of interest, wherein the labeled standard sample comprises a pooled reference sample.

14. The method of claim 1, wherein the step of assessing the biological state of a sample comprises a comparison based at least on a comparison of the absolute concentrations of two or more proteins in one or more of the two or more samples to the concentration of two or more corresponding proteins in one or more of the standard samples.

15. The method of claim 1, wherein the biological state comprises one or more of a disease state, a response to a chemical agent, or combinations thereof.

16. The method of claim 1, wherein:
the transmitted parent ion m/z range of each multiple reaction monitoring scan for a protein of interest listed in column 1 of Table 4 includes an m/z value listed in column 6 of Table 4, which corresponds to a respective signature peptide listed in column 3 of Table 4, which corresponds to a respective protein of interest listed in column 1 of Table 4; and
the transmitted diagnostic daughter ion m/z range of each multiple reaction monitoring scan for a protein of interest listed in column 1 of Table 4 includes an m/z value listed in column 7 of Table 4, which corresponds to a respective parent ion m/z value listed in column 6 of Table 4, which corresponds to a respective signature peptide listed in column 3 of Table 4, which corresponds to a respective protein of interest listed in column 1 of Table 4.


SEQUENCE LISTING (176 sequences)

SEQ ID NO: 1 - protein, 8 aa, Artificial
  synthetic construct
CIGETIGR

SEQ ID NO: 2 - protein, 8 aa, Artificial
  synthetic construct
CIGEIPAK

SEQ ID NO: 3 - protein, 8 aa, Artificial
  synthetic construct
CIGEELSK

SEQ ID NO: 4 - protein, 9 aa, Artificial
  synthetic construct
YCFGEGLAR

SEQ ID NO: 5 - protein, 9 aa, Artificial
  synthetic construct
FCLGESLAK

SEQ ID NO: 6 - protein, 9 aa, Artificial
  synthetic construct
ICLGESIAR

SEQ ID NO: 7 - protein, 9 aa, Artificial
  synthetic construct
ICAGEGLAR

SEQ ID NO: 8 - protein, 9 aa, Artificial
  synthetic construct
VCAGEGLAR

SEQ ID NO: 9 - protein, 9 aa, Artificial
  synthetic construct
ICVGESLAR

SEQ ID NO: 10 - protein, 9 aa, Artificial
  synthetic construct
SCLGEALAR

SEQ ID NO: 11 - protein, 9 aa, Artificial
  synthetic construct
SCLGEPLAR

SEQ ID NO: 12 - protein, 9 aa, Artificial
  synthetic construct
VCVGEGLAR

SEQ ID NO: 13 - protein, 9 aa, Artificial
  synthetic construct
LCLGEPLAR

SEQ ID NO: 14 - protein, 9 aa, Artificial
  synthetic construct
ACLGEQLAK

SEQ ID NO: 15 - protein, 6 aa, Artificial
  synthetic construct
NCLGMR

SEQ ID NO: 16 - protein, 5 aa, Artificial
  synthetic construct
NCIGK

SEQ ID NO: 17 - protein, 18 aa, Artificial
  synthetic construct
YIDLLPTSLPHAVTCDIK

SEQ ID NO: 18 - protein, 9 aa, Artificial
  synthetic construct
ICVGEGLAR

SEQ ID NO: 19 - protein, 9 aa, Artificial
  synthetic construct
ACLGEPLAR

SEQ ID NO: 20 - protein, 8 aa, Artificial
  synthetic construct
CIGEVLAK

SEQ ID NO: 21 - protein, 11 aa, Artificial
  synthetic construct
GFCMFDMECHK

SEQ ID NO: 22 - protein, 9 aa, Artificial
  synthetic construct
ICLGEGIAR

SEQ ID NO: 23 - protein, 8 aa, Artificial
  synthetic construct
LCQNEGCK

SEQ ID NO: 24 - protein, 10 aa, Artificial
  synthetic construct
GCPSLSELWR

SEQ ID NO: 25 - protein, 9 aa, Artificial
  synthetic construct
EECALEIIK

SEQ ID NO: 26 - protein, 10 aa, Artificial
  synthetic construct
GCPSLAEHWK

SEQ ID NO: 27 - protein, 12 aa, Artificial
  synthetic construct
VFANPEDCAFGK

SEQ ID NO: 28 - protein, 9 aa, Artificial
  synthetic construct
SDVVYTDWK

SEQ ID NO: 29 - protein, 15 aa, Artificial
  synthetic construct
YVGGQEHFAHLLILR

SEQ ID NO: 30 - protein, 11 aa, Artificial
  synthetic construct
ATWSGAVLAGR

SEQ ID NO: 31 - protein, 8 aa, Artificial
  synthetic construct
LLELTGPK

SEQ ID NO: 32 - protein, 12 aa, Artificial
  synthetic construct
SGLSTGWTQLSK

SEQ ID NO: 33 - protein, 11 aa, Artificial
  synthetic construct
AIGYLNTGYQR

SEQ ID NO: 34 - protein, 12 aa, Artificial
  synthetic construct
NEDSLVFVQTDK

SEQ ID NO: 35 - protein, 11 aa, Artificial
  synthetic construct
SSGSLLNNAIK

SEQ ID NO: 36 - protein, 10 aa, Artificial
  synthetic construct
ADLSGITGAR

SEQ ID NO: 37 - protein, 19 aa, Artificial
  synthetic construct
AVLDVFEEGTEASAATAVK

SEQ ID NO: 38 - protein, 9 aa, Artificial
  synthetic construct
EIGELYLPK

SEQ ID NO: 39 - protein, 11 aa, Artificial
  synthetic construct
ITLLSALVETR

SEQ ID NO: 40 - protein, 18 aa, Artificial
  synthetic construct
LYGSEAFATDFQDSAAAK

SEQ ID NO: 41 - protein, 12 aa, Artificial
  synthetic construct
AFIQLWAFDAVK

SEQ ID NO: 42 - protein, 11 aa, Artificial
  synthetic construct
TVAACNLPIVR

SEQ ID NO: 43 - protein, 12 aa, Artificial
  synthetic construct
ALQDQLVLVAAK

SEQ ID NO: 44 - protein, 17 aa, Artificial
  synthetic construct
QPFVQGLALYTPVVLPR

SEQ ID NO: 45 - protein, 13 aa, Artificial
  synthetic construct
SLDFTELDVAAEK

SEQ ID NO: 46 - protein, 12 aa, Artificial
  synthetic construct
EVPLNTIIFMGR

SEQ ID NO: 47 - protein, 7 aa, Artificial
  synthetic construct
LQPLDFK

SEQ ID NO: 48 - protein, 11 aa, Artificial
  synthetic construct
TSDQIHFFFAK

SEQ ID NO: 49 - protein, 11 aa, Artificial
  synthetic construct
DLATVYVDVLK

SEQ ID NO: 50 - protein, 13 aa, Artificial
  synthetic construct
DYVSQFEGSALGK

SEQ ID NO: 51 - protein, 14 aa, Artificial
  synthetic construct
LLDNWDSVTSTFSK

SEQ ID NO: 52 - protein, 9 aa, Artificial
  synthetic construct
LSPLGEEMR

SEQ ID NO: 53 - protein, 10 aa, Artificial
  synthetic construct
VQPYLDDFQK

SEQ ID NO: 54 - protein, 22 aa, Artificial
  synthetic construct
AGTELVNFLSYFVELGTQPATQ

SEQ ID NO: 55 - protein, 8 aa, Artificial
  synthetic construct
EQLTPLIK

SEQ ID NO: 56 - protein, 9 aa, Artificial
  synthetic construct
SPELQAEAK

SEQ ID NO: 57 - protein, 10 aa, Artificial
  synthetic construct
ALVQQMEQLR

SEQ ID NO: 58 - protein, 9 aa, Artificial
  synthetic construct
ISASAEELR

SEQ ID NO: 59 - protein, 9 aa, Artificial
  synthetic construct
LEPYADQLR

SEQ ID NO: 60 - protein, 13 aa, Artificial
  synthetic construct
LGEVNTYAGDLQK

SEQ ID NO: 61 - protein, 14 aa, Artificial
  synthetic construct
SELTQQLNALFQDK

SEQ ID NO: 62 - protein, 11 aa, Artificial
  synthetic construct
TQVNTQAEQLR

SEQ ID NO: 63 - protein, 9 aa, Artificial
  synthetic construct
NNALDFVTK

SEQ ID NO: 64 - protein, 13 aa, Artificial
  synthetic construct
SVSLPSLDPASAK

SEQ ID NO: 65 - protein, 12 aa, Artificial
  synthetic construct
YGMVAQVTQTLK

SEQ ID NO: 66 - protein, 9 aa, Artificial
  synthetic construct
EFGNTLEDK

SEQ ID NO: 67 - protein, 9 aa, Artificial
  synthetic construct
EWFSETFQK

SEQ ID NO: 68 - protein, 16 aa, Artificial
  synthetic construct
DALSSVQESQVAQQAR

SEQ ID NO: 69 - protein, 11 aa, Artificial
  synthetic construct
GWVTDGFSSLK

SEQ ID NO: 70 - protein, 17 aa, Artificial
  synthetic construct
SEAEDASLLSFMQGYMK

SEQ ID NO: 71 - protein, 9 aa, Artificial
  synthetic construct
LGPLVEQGR

SEQ ID NO: 72 - protein, 9 aa, Artificial
  synthetic construct
LQAEAFQAR

SEQ ID NO: 73 - protein, 21 aa, Artificial
  synthetic construct
ATFGCHDGYSLDGPEEIECTK

SEQ ID NO: 74 - protein, 9 aa, Artificial
  synthetic construct
ATVVYQGER

SEQ ID NO: 75 - protein, 14 aa, Artificial
  synthetic construct
VCPFAGILENGAVR

SEQ ID NO: 76 - protein, 10 aa, Artificial
  synthetic construct
FQPTLLTLPR

SEQ ID NO: 77 - protein, 14 aa, Artificial
  synthetic construct
LEDMEQALSPSVFK

SEQ ID NO: 78 - protein, 10 aa, Artificial
  synthetic construct
LLDSLPSDTR

SEQ ID NO: 79 - protein, 12 aa, Artificial
  synthetic construct
ALYLQYTDETFR

SEQ ID NO: 80 - protein, 14 aa, Artificial
  synthetic construct
DIASGLIGPLIICK

SEQ ID NO: 81 - protein, 14 aa, Artificial
  synthetic construct
EVGPTNADPVCLAK

SEQ ID NO: 82 - protein, 13 aa, Artificial
  synthetic construct
GAYPLSIEPIGVR

SEQ ID NO: 83 - protein, 9 aa, Artificial
  synthetic construct
DISEVVTPR

SEQ ID NO: 84 - protein, 16 aa, Artificial
  synthetic construct
EAGIPEFYDYDVALIK

SEQ ID NO: 85 - protein, 10 aa, Artificial
  synthetic construct
EELLPAQDIK

SEQ ID NO: 86 - protein, 11 aa, Artificial
  synthetic construct
GDSGGPLIVHK

SEQ ID NO: 87 - protein, 14 aa, Artificial
  synthetic construct
VSEADSSNADWVTK

SEQ ID NO: 88 - protein, 11 aa, Artificial
  synthetic construct
YGLVTYATYPK

SEQ ID NO: 89 - protein, 11 aa, Artificial
  synthetic construct
DGWSAQPTCIK

SEQ ID NO: 90 - protein, 18 aa, Artificial
  synthetic construct
ECDTDGWTNDIPICEVVK

SEQ ID NO: 91 - protein, 10 aa, Artificial
  synthetic construct
LSYTCEGGFR

SEQ ID NO: 92 - protein, 12 aa, Artificial
  synthetic construct
SSNLIILEEHLK

SEQ ID NO: 93 - protein, 10 aa, Artificial
  synthetic construct
WQSIPLCVEK

SEQ ID NO: 94 - protein, 12 aa, Artificial
  synthetic construct
ASSIIDELFQDR

SEQ ID NO: 95 - protein, 11 aa, Artificial
  synthetic construct
ELDESLQVAER

SEQ ID NO: 96 - protein, 17 aa, Artificial
  synthetic construct
LFDSDPITVTVPVEVSR

SEQ ID NO: 97 - protein, 16 aa, Artificial
  synthetic construct
EYVLPSFEVIVEPTEK

SEQ ID NO: 98 - protein, 20 aa, Artificial
  synthetic construct
ILLQGTPVAQMTEDAVDAER

SEQ ID NO: 99 - protein, 14 aa, Artificial
  synthetic construct
IPIEDGSGEVVLSR

SEQ ID NO: 100 - protein, 13 aa, Artificial
  synthetic construct
SSLSVPYVIVPLK

SEQ ID NO: 101 - protein, 9 aa, Artificial
  synthetic construct
TGLQEVEVK

SEQ ID NO: 102 - protein, 13 aa, Artificial
  synthetic construct
GLEEELQFSLGSK

SEQ ID NO: 103 - protein, 10 aa, Artificial
  synthetic construct
LGQYASPTAK

SEQ ID NO: 104 - protein, 15 aa, Artificial
  synthetic construct
VLSLAQEQVGGSPEK

SEQ ID NO: 105 - protein, 9 aa, Artificial
  synthetic construct
AEFQDALEK

SEQ ID NO: 106 - protein, 12 aa, Artificial
  synthetic construct
LNMGITDLQGLR

SEQ ID NO: 107 - protein, 9 aa, Artificial
  synthetic construct
VDFTLSSER

SEQ ID NO: 108 - protein, 10 aa, Artificial
  synthetic construct
VGDTLNLNLR

SEQ ID NO: 109 - protein, 12 aa, Artificial
  synthetic construct
AIEDYINEFSVR

SEQ ID NO: 110 - protein, 11 aa, Artificial
  synthetic construct
LSPIYNLVPVK

SEQ ID NO: 111 - protein, 9 aa, Artificial
  synthetic construct
VVEESELAR

SEQ ID NO: 112 - protein, 14 aa, Artificial
  synthetic construct
EHAVEGDCDFQLLK

SEQ ID NO: 113 - protein, 7 aa, Artificial
  synthetic construct
FSVVYAK

SEQ ID NO: 114 - protein, 10 aa, Artificial
  synthetic construct
HTLNQIDEVK

SEQ ID NO: 115 - protein, 11 aa, Artificial
  synthetic construct
DLQFVEVTDVK

SEQ ID NO: 116 - protein, 11 aa, Artificial
  synthetic construct
IYLYTLNDNAR

SEQ ID NO: 117 - protein, 10 aa, Artificial
  synthetic construct
STTPDITGYR

SEQ ID NO: 118 - protein, 12 aa, Artificial
  synthetic construct
WLPSSSPVTGYR

SEQ ID NO: 119 - protein, 13 aa, Artificial
  synthetic construct
AGALNSNDAFVLK

SEQ ID NO: 120 - protein, 22 aa, Artificial
  synthetic construct
AQPVQVAEGSEPDGFWEALGGK

SEQ ID NO: 121 - protein, 8 aa, Artificial
  synthetic construct
TASDFITK

SEQ ID NO: 122 - protein, 9 aa, Artificial
  synthetic construct
DYFMPCPGR

SEQ ID NO: 123 - protein, 15 aa, Artificial
  synthetic construct
GECQAEGVLFFQGDR

SEQ ID NO: 124 - protein, 11 aa, Artificial
  synthetic construct
GGYTLVSGYPK

SEQ ID NO: 125 - protein, 21 aa, Artificial
  synthetic construct
LLQDEFPGIPSPLDAAVECHR

SEQ ID NO: 126 - protein, 11 aa, Artificial
  synthetic construct
NFPSPVDAAFR

SEQ ID NO: 127 - protein, 9 aa, Artificial
  synthetic construct
VDGALCMEK

SEQ ID NO: 128 - protein, 11 aa, Artificial
  synthetic construct
YYCFQGNQFLR

SEQ ID NO: 129 - protein, 7 aa, Artificial
  synthetic construct
IAIDLFK

SEQ ID NO: 130 - protein, 9 aa, Artificial
  synthetic construct
NYNLVESLK

SEQ ID NO: 131 - protein, 9 aa, Artificial
  synthetic construct
TLEAQLTPR

SEQ ID NO: 132 - protein, 9 aa, Artificial
  synthetic construct
DGYLFQLLR

SEQ ID NO: 133 - protein, 14 aa, Artificial
  synthetic construct
DSPVLIDFFEDTER

SEQ ID NO: 134 - protein, 14 aa, Artificial
  synthetic construct
GGEGTGYFVDFSVR

SEQ ID NO: 135 - protein, 8 aa, Artificial
  synthetic construct
ELAAQTIK

SEQ ID NO: 136 - protein, 18 aa, Artificial
  synthetic construct
FAHYVVTSQVVNTANEAR

SEQ ID NO: 137 - protein, 10 aa, Artificial
  synthetic construct
LDAQASFLPK

SEQ ID NO: 138 - protein, 8 aa, Artificial
  synthetic construct
NDLISATK

SEQ ID NO: 139 - protein, 9 aa, Artificial
  synthetic construct
SLAPTAAAK

SEQ ID NO: 140 - protein, 10 aa, Artificial
  synthetic construct
SSALDMENFR

SEQ ID NO: 141 - protein, 10 aa, Artificial
  synthetic construct
TEVNVLPGAK

SEQ ID NO: 142 - protein, 11 aa, Artificial
  synthetic construct
VQFELHYQEVK

SEQ ID NO: 143 - protein, 19 aa, Artificial
  synthetic construct
VVNNSPQPQNVVFDVQIPK

SEQ ID NO: 144 - protein, 9 aa, Artificial
  synthetic construct
GSEMVVAGK

SEQ ID NO: 145 - protein, 8 aa, Artificial
  synthetic construct
ILDDLSPR

SEQ ID NO: 146 - protein, 8 aa, Artificial
  synthetic construct
NVVFVIDK

SEQ ID NO: 147 - protein, 19 aa, Artificial
  synthetic construct
DIPTNSPELEETLTHTITK

SEQ ID NO: 148 - protein, 11 aa, Artificial
  synthetic construct
ENFLFLTPDCK

SEQ ID NO: 149 - protein, 11 aa, Artificial
  synthetic construct
TVGSDTFYSFK

SEQ ID NO: 150 - protein, 8 aa, Artificial
  synthetic construct
YFIDFVAR

SEQ ID NO: 151 - protein, 10 aa, Artificial
  synthetic construct
EAQLPVIENK

SEQ ID NO: 152 - protein, 19 aa, Artificial
  synthetic construct
EPLDDYVNTQGASLFSVTK

SEQ ID NO: 153 - protein, 10 aa, Artificial
  synthetic construct
LSSPAVITDK

SEQ ID NO: 154 - protein, 13 aa, Artificial
  synthetic construct
QLGAGSIEECAAK

SEQ ID NO: 155 - protein, 8 aa, Artificial
  synthetic construct
WELCDIPR

SEQ ID NO: 156 - protein, 14 aa, Artificial
  synthetic construct
EVQPVELPNCNLVK

SEQ ID NO: 157 - protein, 7 aa, Artificial
  synthetic construct
STVELFK

SEQ ID NO: 158 - protein, 11 aa, Artificial
  synthetic construct
DPNGLPPEAQK

SEQ ID NO: 159 - protein, 18 aa, Artificial
  synthetic construct
LLNLDGTCADSYSFVFSR

SEQ ID NO: 160 - protein, 10 aa, Artificial
  synthetic construct
YWGVASFLQK

SEQ ID NO: 161 - protein, 12 aa, Artificial
  synthetic construct
ETAASLLQAGYK

SEQ ID NO: 162 - protein, 9 aa, Artificial
  synthetic construct
ETWTANVGK

SEQ ID NO: 163 - protein, 13 aa, Artificial
  synthetic construct
AADDTWEPFASGK

SEQ ID NO: 164 - protein, 13 aa, Artificial
  synthetic construct
GSPAINVAVHVFR

SEQ ID NO: 165 - protein, 23 aa, Artificial
  synthetic construct
YTIAALLSPYSYSTTAVVTNPKE

SEQ ID NO: 166 - protein, 22 aa, Artificial
  synthetic construct
HQPQEFPTYVEPTNDEICEAFR

SEQ ID NO: 167 - protein, 8 aa, Artificial
  synthetic construct
LCDNLSTK

SEQ ID NO: 168 - protein, 20 aa, Artificial
  synthetic construct
SCESNSPFPVHPGTAECCTK

SEQ ID NO: 169 - protein, 18 aa, Artificial
  synthetic construct
SLGECCDVEDSTTCFNAK

SEQ ID NO: 170 - protein, 11 aa, Artificial
  synthetic construct
VCSQYAAYGEK

SEQ ID NO: 171 - protein, 15 aa, Artificial
  synthetic construct
DVWGIEGPIDAAFTR

SEQ ID NO: 172 - protein, 16 aa, Artificial
  synthetic construct
DWHGVPGQVDAAMAGR

SEQ ID NO: 173 - protein, 10 aa, Artificial
  synthetic construct
GQYCYELDEK

SEQ ID NO: 174 - protein, 15 aa, Artificial
  synthetic construct
SIAQYWLGCPAPGHL

SEQ ID NO: 175 - protein, 18 aa, Artificial
  synthetic construct
GAGTGGLGLAVEGPSEAK

SEQ ID NO: 176 - protein, 14 aa, Artificial
  synthetic construct
LQAAGIQLHNVWAR